(19) United States
(12) Patent Application Publication
(10) Pub. No.: US 2017/0042857 A1
(43) Pub. Date: Feb. 16, 2017

(54) SYNAPTOJANIN-2 INHIBITORS FOR USE IN THE TREATMENT OF CANCER

(71) Applicant: YEDA RESEARCH AND DEVELOPMENT CO. LTD., Rehovot (IL)

(72) Inventors: Yosef YARDEN, Rehovot (IL); Nir BEN-CHETRIT, Rehovot (IL)

(21) Appl. No.: 15/100,322

(22) PCT Filed: Nov. 27, 2014
(86) PCT No.: PCT/IB2014/066404

§ 371 Date: May 30, 2016

Related U.S. Application Data
(63) Continuation-in-part of application No. PCT/IL2013/050986, filed on Nov. 28, 2013.
(60) Provisional application No. 61/910,086, filed on Nov. 28, 2013.
(60) Provisional application No. 61/731,003, filed on Nov. 29, 2012.

(51) Int. Cl. A61K 31/353; A61K 31/7048; A61K 31/7024; A61K 31/12; A61K 45/06; A61K 31/155; A61K 31/4709; A61K 31/216; A61K 31/235; A61K 31/05; A61K 31/7028; A61K 31/7032

(52) U.S. Cl. CPC A61K 31/353; A61K 31/7028; A61K 31/7048; A61K 31/7024; A61K 31/12; A61K 31/7032; A61K 31/155; A61K 31/4709; A61K 31/216; A61K 31/235; A61K 31/05; A61K 45/06

(57) ABSTRACT

Disclosed herein are synaptojanin-2 inhibitors, and novel methods and uses utilizing same for preventing tumor metastasis, treating cancer or inhibiting synaptojanin-2. Compounds disclosed herein include chlorhexidine and pyrvinium, the compound having the formula:

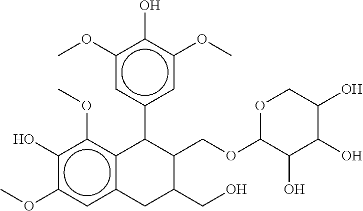

and compounds characterized by the general formula:

X-L-[(Y)i-(Z)j]-(L-X)k

and/or by the general formula:

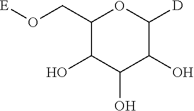

wherein L, X, Y, Z, D, E, i, j and k are as defined herein.

### FIELD AND BACKGROUND OF THE INVENTION

The present invention, in some embodiments thereof, relates to cancer therapy and more particularly, but not exclusively, to compounds, compositions and methods for preventing tumor metastasis, and for treating cancer.

Cell motility supports a variety of physiological and pathological processes, including tumor metastasis [Ridley, Cell 145, 1012-1022 (2011)]. The onset of migration is driven by actin polymerization and Rho-family GTPases, which instigate formation of lamellipodia and filopodia. Burgeoning evidence implicates another type of actin-driven protrusions, called invadopodia, in matrix degradation [Murphy & Courtneidge, Nat Rev Mol Cell Biol 12, 413-426 (2011)]. To seed metastases, migratory breast cancer cells form invadopodia and infiltrate into nearby vessels.

Intracellular trafficking emerges as a key feature of cell migration and tumor progression [Mosesson et al., Nat Rev Cancer 8, 835-850 (2008)]. Phosphoinositides play pivotal roles in cellular compartmentalization by determining vesicle identity [Yuan & Cantley, Oncogene 27, 5497-5510 (2008)]. For example, phosphorylation at the D3 position of PI(4,5)P₂ (phosphatidyl-inositol 4,5-bisphosphate) by phosphatidylinositol 3-kinase (PI3K) generates PI(3,4,5)P₃, which is necessary for invadopodia formation [Yamaguchi et al., J Cell Biology 193, 1275-1288 (2011)]. Similarly, PI(4,5)P₂ regulates multiple proteins controlling endocytosis and actin dynamics [Saarikangas et al., Physiol Rev 90, 259-289 (2010)], but its levels are stringently controlled by two additional types of enzymes: phospholipase C (PLCγ) promotes PI(4,5)P₂ hydrolysis, which activates cofilin (an actin-severing protein) and drives mammary cell migration [van Rheenen et al., J Cell Biology 179, 1247-1259 (2007)], and synaptojanin-2.

Synaptojanin-2 (SYNJ2) is an inositol polyphosphate 5-phosphatase, which dephosphorylates the D5 position of the inositol ring. Dephosphorylation by SYNJ2 controls glioma cell migration [Chuang et al., Cancer Research 64, 8271-8275 (2004); Malecz et al., Curr Biol 10, 1383-1386 (2000)]. In addition homozygous mutations in SYNJ2 were identified in certain prostate cancer samples [Rossi et al., Cancer Genet Cytogenet 161, 97-103 (2005)].

The flavonoid ampelopsin has been reported to inhibit growth and metastasis of prostate cancer [Ni et al., PLoS ONE 7, e38802 (2012)]. The flavonoid chrysin has been reported to suppress survival and metastasis of mouse breast cancer cells [Lirdprapamongkol et al., Oncol Rep 30, 2357-2364 (2013)]. Additional flavonoids for which an anti-invasive or anti-metastatic activity towards tumors has been reported include (−)-epigallocatechin-3-gallate, (−)-epigallocatechin, (−)-epicatechin-3-gallate, (−)-epicatechin, genistein/genistin, silibinin, nobiletin, quercetin, anthocyanin, luteolin, apigenin, myricetin, tangeritin, kaempferol, glycitein, licoricidin, daidzein and naringenin [Weng & Yen, Cancer Metastasis Rev 31, 323-351 (2012); Kawabata et al., Biosci Biotechnol Biochem 69, 307-314 (2005)].

International PCT Patent Application PCT/IL2013/050986 (published as WO2014/083567), the contents of which are herein incorporated by reference in their entirety, presents evidence that SYNJ2 plays a major role in regulating cell migration and tumor metastasis, and that inhibition of SYNJ2 activity in cancer cells results in a considerable loss of metastatic potential.

Additional background art includes Muller et al. [Cell 139, 1327-1341 (2010)]; Minn et al. [Nature 436, 518-524 (2005)]; and Bos et al. [Nature 459, 1005-1009 (2009)].

### SUMMARY OF THE INVENTION

According to an aspect of some embodiments of the invention, there is provided a method of preventing tumor metastasis, the method comprising administering to a subject in need thereof a therapeutically effective amount of a compound having the general formula I:

X-L-[(Y)i-(Z)j]-(L-X)k Formula I

or a pharmaceutically acceptable salt thereof, wherein:

i, j and k are each independently 0 or 1, wherein at least one of i, j and k is 1;

L is absent or is a linking moiety;

X is an aryl group substituted by one or more group selected from the group consisting of hydroxy, thiohydroxy, alkoxy, aryloxy, thioalkoxy and thioaryloxy;

Z is selected from the group consisting of a monosaccharide moiety, a disaccharide moiety, a shikimate moiety and a quinate moiety; and

Y is a bicyclic moiety having the general formula II:

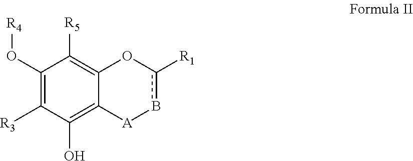

wherein:

A is absent or is CH₂, C═O, C═S or C═NR₆;

B is absent or is O, S, NR₇, CH, CH₂, C—O—R₂, C—S—R₂, C—N(R₈)—R₂, CH—O—R₂, CH—S—R₂ or CH—N(R₉)—R₂;

R₁-R₅ are each independently selected from the group consisting of hydrogen, methyl, aryl and a covalent bond with an L, Z or X moiety described herein;

R₆-R₉ are each independently selected from the group consisting of hydrogen and alkyl, and

the dashed line denotes a saturated or unsaturated bond, wherein when the dashed line denotes a saturated bond, B is O, CH₂ or CH—O—R₂, and when the dashed line denotes an unsaturated bond, B is CH or C—O—R₂,

thereby preventing tumor metastasis.

According to an aspect of some embodiments of the invention, there is provided a method of preventing tumor metastasis, the method comprising administering to a subject in need thereof a therapeutically effective amount of a compound having the general formula III:

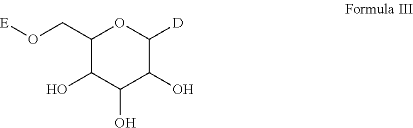

or a pharmaceutically acceptable salt thereof, wherein:

D is selected from the group consisting of:

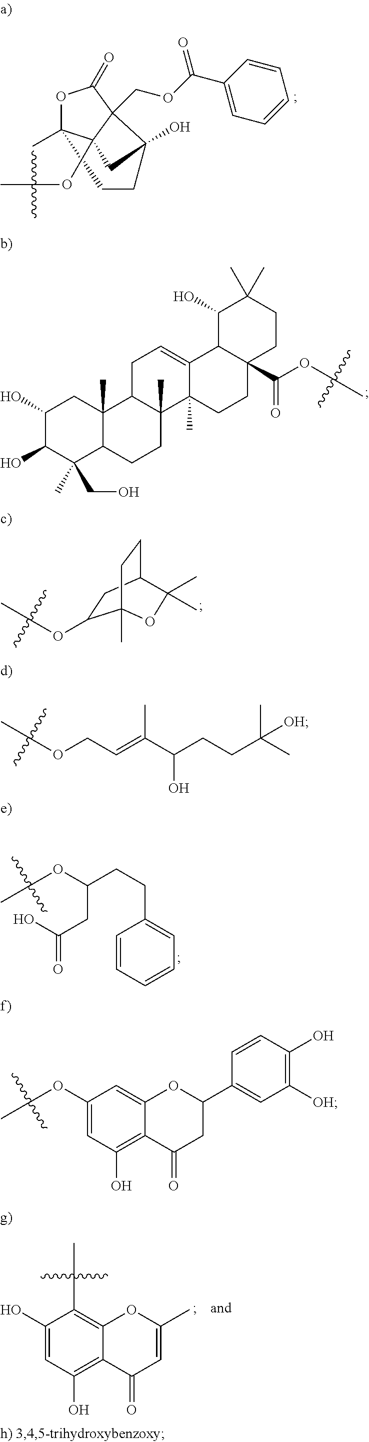

and

E is selected from the group consisting of hydrogen and substituted or non-substituted benzyl,

thereby preventing tumor metastasis.

According to an aspect of some embodiments of the invention, there is provided a method of preventing tumor metastasis, the method comprising administering to a subject in need thereof a therapeutically effective amount of a compound listed in Table 1 herein, or a pharmaceutically acceptable salt thereof, thereby preventing tumor metastasis.

According to an aspect of some embodiments of the invention, there is provided a method of preventing tumor metastasis, the method comprising administering to a subject in need thereof a therapeutically effective amount of a compound selected from the group consisting of Compound 12 in Table 1 herein, chlorhexidine and pyrvinium, and pharmaceutically acceptable salts thereof, thereby preventing tumor metastasis.

According to an aspect of some embodiments of the invention, there is provided a compound having the general formula I hereinabove, for preventing tumor metastasis.

According to an aspect of some embodiments of the invention, there is provided a compound having the general formula III hereinabove, for preventing tumor metastasis.

According to an aspect of some embodiments of the invention, there is provided a compound listed in Table 1 herein, or a pharmaceutically acceptable salt thereof, for preventing tumor metastasis.

According to an aspect of some embodiments of the invention, there is provided a compound selected from the group consisting of Compound 12 in Table 1 herein, chlorhexidine and pyrvinium, and pharmaceutically acceptable salts thereof, for preventing tumor metastasis.

According to an aspect of some embodiments of the invention, there is provided a method of treating cancer the method comprising, administering to a subject in need thereof a therapeutically effective amount of a compound having the general formula I hereinabove, and an inhibitor of a cell surface receptor associated with an onset or progression of cancer, thereby treating cancer.

According to an aspect of some embodiments of the invention, there is provided a method of treating cancer the method comprising, administering to a subject in need thereof a therapeutically effective amount of a compound having the general formula III hereinabove, and an inhibitor of a cell surface receptor associated with an onset or progression of cancer, thereby treating cancer.

According to an aspect of some embodiments of the invention, there is provided a method of treating cancer the method comprising, administering to a subject in need thereof a therapeutically effective amount of a compound listed in Table 1 herein or a pharmaceutically acceptable salt thereof, and an inhibitor of a cell surface receptor associated with an onset or progression of cancer, thereby treating cancer.

According to an aspect of some embodiments of the invention, there is provided a method of treating cancer the method comprising, administering to a subject in need thereof a therapeutically effective amount of:

a compound selected from the group consisting of Compound 12 in Table 1 herein, chlorhexidine and pyrvinium, and pharmaceutically acceptable salts thereof, and

an inhibitor of a cell surface receptor associated with an onset or progression of cancer, thereby treating cancer.

According to an aspect of some embodiments of the invention, there is provided a compound having the general formula I hereinabove, for treating cancer.

According to an aspect of some embodiments of the invention, there is provided a compound having the general formula III hereinabove, and an inhibitor of a cell surface receptor associated with an onset or progression of cancer, for treating cancer.

According to an aspect of some embodiments of the invention, there is provided a compound listed in Table 1 herein or a pharmaceutically acceptable salt thereof and an inhibitor of a cell surface receptor associated with an onset or progression of cancer, for treating cancer.

According to an aspect of some embodiments of the invention, there is provided a compound selected from the group consisting of Compound 12 in Table 1 herein, chlorhexidine and pyrvinium, and pharmaceutically acceptable salts thereof,

and an inhibitor of a cell surface receptor associated with an onset or progression of cancer, for treating cancer.

According to an aspect of some embodiments of the invention, there is provided a kit for the treatment of cancer or prevention of cancer metastasis, comprising a packaging material packaging a compound as described herein and an inhibitor of a cell surface receptor associated with an onset or progression of cancer.

According to an aspect of some embodiments of the invention, there is provided a method of inhibiting synaptojanin-2, the method comprising contacting the synaptojanin-2 with an effective amount of a compound having the general formula I hereinabove, thereby inhibiting synaptojanin-2.

According to an aspect of some embodiments of the invention, there is provided a method of inhibiting synaptojanin-2, the method comprising contacting the synaptojanin-2 with an effective amount of a compound having the general formula III hereinabove, thereby inhibiting synaptojanin-2.

According to an aspect of some embodiments of the invention, there is provided a method of inhibiting synaptojanin-2, the method comprising contacting the synaptojanin-2 with an effective amount of a compound listed in Table 1 herein or a pharmaceutically acceptable salt thereof, thereby inhibiting synaptojanin-2.

According to an aspect of some embodiments of the invention, there is provided a method of inhibiting synaptojanin-2, the method comprising contacting the synaptojanin-2 with an effective amount of a compound selected from the group consisting of Compound 12 in Table 1 herein, chlorhexidine and pyrvinium, and pharmaceutically acceptable salts thereof, thereby inhibiting synaptojanin-2.

According to some embodiments, the abovementioned method is effected ex vivo.

According to some embodiments, the abovementioned method is for treating a disease or disorder in which inhibition of synaptojanin-2 is beneficial.

According to some embodiments, the metastasis is EGF dependent.

According to some embodiments, the tumor is a breast cancer tumor.

According to some embodiments, the cell surface receptor associated with the onset or progression of cancer is a receptor tyrosine kinase.

According to some embodiments, the receptor tyrosine kinase is an ErbB receptor.

According to some embodiments, the ErbB receptor is Epidermal Growth Factor Receptor (EGFR).

According to some embodiments, the cancer is breast cancer.

According to some embodiments, the inhibitor of the cell surface receptor associated with the onset or progression of cancer is an antibody.

According to some embodiments, the inhibitor of the cell surface receptor associated with the onset or progression of cancer is a small molecule inhibitor.

According to some embodiments, A is CH₂ or C═O.

According to some embodiments, B is CH, CH₂, C—O—R₂, or CH—O—R₂.

According to some embodiments, L is absent or is a linking moiety selected from the group consisting of C(═O), CH═CH, CH═CH—C(═O) and CH₂.

According to some embodiments, the aryl is a phenyl.

According to some embodiments, X is a hydroxylated phenyl group selected from the group consisting of trihydroxyphenyl, dihydroxyphenyl, hydroxyphenyl, methoxydihydroxyphenyl, and methoxyhydroxyphenyl;

According to some embodiments, X is selected from the group consisting of 3,4,5-trihydroxyphenyl, 3,4-dihydroxyphenyl, 3,5-dihydroxyphenyl, 3,5-dihydroxyphenyl, 2,3-dihydroxyphenyl, 6-methoxy-2,4-dihydroxyphenyl, 3-methoxy-4-hydroxyphenyl, 3-hydroxy-4-methoxyphenyl and 4-hydroxyphenyl.

According to some embodiments, i is 1.

According to some embodiments, R₁ is selected from the group consisting of methyl, phenyl and a covalent bond.

According to some embodiments, R₁ is a bond with X, or R₁ is phenyl.

According to some embodiments, R₂ is selected from the group consisting of hydrogen and a covalent bond.

According to some embodiments, R₃ is selected from the group consisting of hydrogen, methyl and a covalent bond.

According to some embodiments, R₄ and R₅ are each independently selected from the group consisting of hydrogen and a covalent bond.

According to some embodiments, the dashed line denotes a saturated bond, the carbon atom attached to R₁ is chiral and is in an (S) configuration, and the carbon atom of B is chiral, and is an (S) configuration when A is C═O, and in an (R) configuration when A is CH₂.

According to some embodiments, j is 0, and X is selected from the group consisting of 3,4,5-trihydroxyphenyl, 3,4-dihydroxyphenyl, 2,3-dihydroxyphenyl and 4-hydroxyphenyl.

According to some embodiments, k is 1, and L is absent or is C(═O) or CH₂.

According to some embodiments, R₁ and R₄ are each independently a covalent bond to Z, L or X.

According to some embodiments, Z is a monosaccharide or disaccharide, being attached to Y, L or X via a glycosidic bond.

According to some embodiments, i is 0.

According to some embodiments, j is 0 and k is 1.

According to some embodiments, each X is independently selected from the group consisting of 2,4-dihydroxyphenyl, 3,4-dihydroxyphenyl, 3,5-dihydroxyphenyl and 6-methoxy-2,4-dihydroxyphenyl.

According to some embodiments, L is selected from the group consisting of C(═O) and CH═CH.

According to some embodiments, j is 1.

According to some embodiments, Z is selected from the group consisting of a monosaccharide, shikimate and quinate.

According to some embodiments, the monosaccharide is attached to L or X at the 1-position and/or 6-position of the monosaccharide, and the shikimate and the quinate are attached to L and/or X at a 3-position and/or 5-position of the shikimate or the quinate.

According to some embodiments, a sum of i, j and k is 2.

According to some embodiments, the compound has the general formula:

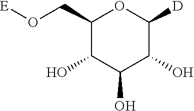

or a pharmaceutically acceptable salt thereof.

According to some embodiments, D is a terpenoid derivative selected from the group consisting of:

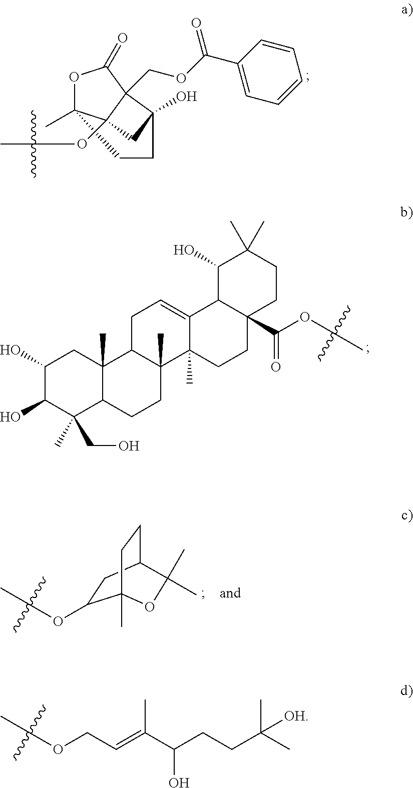

According to some embodiments, E is hydrogen.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-I show that EGF promotes invasive growth of mammary cells and induces a specific set of genes. FIG. 1A—MCF10A cells were plated in the absence of growth factors and allowed to form clusters. Seventy-two hours later, cells were treated with the indicated growth factors (each at 10 ng/mL) and phase contrast images were taken 24 hours later (scale bar, 50 μm). FIG. 1B—MCF10A cells were plated in migration or invasion chambers, as indicated, in the presence of the indicated ligands (10 ng/mL), and 18 hours later cells that migrated to the lower compartment were stained with crystal violet (left panel). Shown are quantifications of migration and invasion signals, normalized to the effect of EGF treatment. Data represent mean±S.D. of biological triplicates from a representative experiment that was repeated twice (right panel). FIG. 1C—MCF10A cells were plated in transwell inserts in EGF-containing medium, without or with the inhibitors AG-1478 (1 μM), U0126 (5 μM), or Wortmannin (200 nM), and allowed to migrate for 18 hours. Data represent mean±S.D. of triplicates. The experiment was repeated twice. FIG. 1D—A list of 425 genes specifically induced in human mammary MCF10A cells by EGF (and not by serum) [Amit et al., Nat Genet 39, 503-512 (2007)], was intersected with genes that were up-regulated in the context of metastasis of MDA-MB-231 cells (1,597 genes) [Minn et al., Nature 436, 518-524 (2005)]. One of the 23 overlapping genes encodes the 5′-phosphatidylinositol lipid phosphatase Synaptojanin-2 (SYNJ2). FIG. 1E—MCF10A cells were infected with lentiviral particles encoding LacZ (Ctrl) or SYNJ2-GFP (SYNJ2-OX). Expression levels of the endogenous SYNJ2 and the SYNJ2-GFP fusion protein were determined by immunoblotting, and equal protein loading was confirmed by probing for tubulin. FIG. 1F—The Ctrl and SYNJ2-OX clones of MCF10A cells were plated in migration chambers (5×10⁴ cells/well) in the absence (NT) or presence of EGF (10 ng/mL) and allowed to migrate for 22 hours. Migrating cells that reached the other side of the filter were stained with crystal violet and images were taken. FIG. 1G—MCF10A cells were transfected with siRNA control (siCtrl) or siRNA directed to SYNJ2 (siSYNJ2), and protein levels of SYNJ2 were determined 36 hours later by immunoblotting. Equal protein loading was confirmed by immunoblotting for Ras-GAP. FIG. 1H—The cells presented in G were plated in migration chambers (5×10⁴ cells/well) in the absence (NT) or presence of EGF (10 ng/mL) and allowed to migrate for 22 hours. Migrating cells that reached the lower face of the filter were stained with crystal violet and images were captured. FIG. 1I—Confluent cultures of MCF10A cells were treated with the indicated siRNAs. Once monolayers formed, they were subjected to an automated scratching system that monitors the rate of scratch closure.

FIGS. 2A-E show that transcriptional induction of SYNJ2 by EGF promotes invasive growth. FIG. 2A—Serum-starved MCF10A cells were stimulated with EGF (20 ng/mL) or serum (5%), and SYNJ2 mRNA expression was assayed by using microarrays or RT-qPCR. FIG. 2B—MCF10A cells were stimulated with EGF, extracted and immunoblotted as indicated. FIG. 2C—MCF10A cells, infected with viruses encoding GFP-SYNJ2 (SYNJ2-OX) or LacZ as control (Ctrl), were cultured for 4 days in the absence or presence of EGF. Phase contrast (top, bar: 100 μm) and confocal images (bottom, bar: 20 μm) using phalloidin and DAPI were obtained. FIGS. 2D-E—MCF10A cells were cultured for 22 hours in migration or invasion chambers (5−6×10⁴ cells/well) in the absence (NT) or presence of EGF (10 ng/mL). Cells that reached the filter's bottom were stained and filter's coverage quantified (mean±S.D.).

FIGS. 3A-G show inducible translocation of SYNJ2 to the leading edge accompanies mammary cell migration and invasion. FIG. 3A—MDA-MB-231 cells were infected with lentiviral particles encoding LacZ (Ctrl) or a V5-tagged SYNJ2 (SYNJ2-V5), along with control shRNA (shCtrl) or an shRNA directed against SYNJ2 (shSYNJ2). Protein levels of V5-SYNJ2 and endogenous SYNJ2 were determined by immunoblotting. Equal protein loading was confirmed by immunoblotting for AKT. FIG. 3B—Phase images (left panels) and invasion images (right panels) of MDA-MB-231 cells stably over-expressing SYNJ2, or LacZ as control. The invasive capacities were determined in triplicates using an invasion assay, and invading cells were quantified and normalized to control (Ctrl). Scale bar, 50 μm. FIG. 3C—MDA-MB-231 cells were transfected with siRNA oligonucleotides directed to SYNJ2 (or siCtrl). Following 36 hours, protein levels of SYNJ2 were determined by immunoblotting. Equal protein loading was confirmed by immunoblotting for Ras-GAP. FIG. 3D—Cells from C were plated in migration or invasion chambers and incubated for 18 hours. The migration and invasion signals were quantified and normalized to EGF-treated siCtrl cells. Data shown are means±S.D. of triplicates. FIG. 3E—MDA-MB-231 cells transiently expressing GFP-SYNJ2 were plated on glass coverslips and stimulated with TGFα (10 ng/mL). Time-lapse microscopy photos were taken (every 10 seconds). The images shown are inverted, with black spots representing SYNJ2 and its assembly at the base of lamellipodia. Scale bar, 10 μm. FIG. 3F—MDA-MB-231 cells were immunostained for endogenous SYNJ2 and F-actin using TRITC-phalloidin. The squared area is magnified. Scale bar, 10 μm. FIG. 3G—MCF10A cells were stimulated with EGF for 18 hours, and then immunostained for endogenous SYNJ2 and counter-stained for F-actin using TRITC-phalloidin. Scale bar, 10 μm.

FIGS. 4A-F show that the catalytic activity of SYNJ2 is essential for invasive growth. FIGS. 4A-B—MDA-MB-231 cells expressing SYNJ2 (SYNJ2-OX) or shRNA to SYNJ2 (shSYNJ2), as well as control cells, were seeded in 5% Matrigel. Images were captured after six days, and invasive spheroids quantified (mean±S.D.). Scale bars, 50 μm. FIGS. 4C-D—shSYNJ2-expressing MDA-MB-231 cells were infected with WT SYNJ2 (shSYNJ2+SYNJ2^{WT}) or with a catalytically disabled mutant (shSYNJ2+SYNJ2^{CD}). Cells were either extracted and immunoblotted as indicated, or they were allowed to invade for 18 hours in invasion chambers. Images of the invaded cells and their normalized quantification are shown (mean±S.D). FIG. 4E—show scanning electron micrographs of shCtrl and shSYNJ2 cells grown on fibronectin. Scale bar, 2 μm. FIG. 4F—Images of F-actin in the indicated MDA-MB-231 cells stained with phalloidin and DAPI. Z-axis sections (lines) and magnified areas are shown. Arrowheads mark swollen structures. Scale bar, 10 μm.

FIGS. 5A-H show the subcellular localization of SYNJ2. FIG. 5A—MDA-MB-231 cells expressing GFP-SYNJ2 were transfected with an RFP-Clathrin and plated on fibronectin-coated plates. Using spinning-disc microscopy, cells were imaged every five seconds. Arrowheads mark a newly formed leading edge. Scale bar, 5 μm. FIG. 5B—Representative time frames depicting assembly and disassembly of SYNJ2 at the leading edge (upper two rows) and underneath the cell body. For the lower rows, cells were transfected with a mCherry-lifeACT plasmid and plated on collagen. Thereafter, cells were imaged at 1 minute intervals. Arrowheads were inserted for reference. Note the difference in time scales. Scale bar, 1 μm. FIG. 5C—Cells were simultaneously imaged by TIRF and epifluorescence microscopy and signals converted into kymographs (x-axis). Arrowheads mark signal initiation. Scale bar, 5 μm. FIG. 5D—Cells were imaged using spinning disc confocal microscopy 5 minutes before and 5 minutes after treatment with Dyngo-4a (30 μM; a Dynamin-2 inhibitor). Scale bar, 5 μm. FIG. 5E—MDA-MB-231 cells stably expressing GFP-SYNJ2 were pre-incubated with Dyngo-4a (30 μM; 30 min), or with solvent (DMSO). Cell lysates were subjected to immunoprecipitation with anti-GFP antibodies (or with no antibody; -Ab), and then immunoblotted, along with a sample (5%) of the cell lysate, with the indicated antibodies. FIG. 5F—Cells were plated on fibronectin, fixed and immunostained for endogenous Rac1. Scale bar, 10 μm. FIG. 5G—Cells were imaged using confocal microscopy 5 minutes prior to and 5 minutes after a 30 min-long treatment with NSC-23766 (5 μM). Scale bar, 5 μm. FIG. 5H—MDA-MB-231 cells were treated with the indicated siRNA oligonucleotides. Cell extracts were blotted for SYNJ2 and Ras-GAP. GTP-Rac1 levels were determined using an ELISA-based assay (Cytoskeleton).

FIGS. 6A-D show SYNJ2 localization to the leading edge is distinct from caveolins distribution and depends on F-actin, cholesterol and PI3K. FIG. 6A—MDA-MB-231 cells expressing GFP-SYNJ2 and co-expressing RFP-Cav1 were simultaneously imaged over time, and signals converted into kymographs (x- and y-axis). Note the transient nature of SYNJ2 assemblies and stable appearance of Caveolin 1. Scale bar, 5 μm. FIG. 6B—The left panel depicts the distribution (% of pits versus lifetime) of 150 randomly selected SYNJ2 assemblies, imaged as in FIG. 5A (5 second intervals, single plane, spinning disk confocal). The right panel depicts the average (±SEM) relative intensity of assemblies that showed a 55 seconds lifetime. FIG. 6C—MDA-MB-231 cells stably-expressing GFP-SYNJ2 were treated with MβCD (10 mM, 15 minutes) or with Wortmannin (500 nM, 15 minutes). Images of the same selected cells were captured every 6 seconds, either prior to or following treatment, and signals were converted into kymographs (representing the squared insets in the left panels). Scale bar, 20 μm. FIG. 6D—MDA-MB-231 cells stably co-expressing GFP-SYNJ2 and lifeACT-mCherry were treated with LatrunculinB (1 μM, 15 minutes). Images were acquired either prior to or following treatment. Scale bar, 5 μm.

FIGS. 7A-E show SYNJ2 depletion arrests EGFR in intracellular vesicles FIG. 7A—MCF10A cells stably expressing shRNA control (shCtrl) or shRNA specific to SYNJ2 (shSYNJ2) were extracted three days after plating in EGF-containing medium. Immunoblots were probed for SYNJ2, EGFR, phosphorylated tyrosine 1068 of EGFR (pEGFR), phosphorylated ERK (pERK), and Ras-GAP, as a loading control. FIG. 7B—MCF10A cells were transfected with siRNA control, or siRNA directed against SYNJ2, in the presence of EGF. Confocal immunofluorescence analysis was performed using EGFR and SYNJ2 antibodies. Note that only the SYNJ2-depleted cell (asterisk) displays EGFR trafficking defects. Scale bar, 10 μm. FIG. 7C—Three derivatives of MDA-MB-231 cells were immunostained for EGFR and counterstained for DAPI and F-actin: (i) cells in which SYNJ2 was knocked-down (shSYNJ2; left column), (ii) the same cells infected by lentiviral gene transfer corresponding to the catalytically-dead form (shSYNJ2+SYNJ2^{CD}; middle column), and (iii) cells in which SYNJ2 was knocked-down and the wild type form was introduced by infection (shSYNJ2+SYNJ2^{WT}; right column). Scale bar, 20 μm. FIG. 7D—Ubiquitinated EGFR levels (densitometry). FIG. 7E—MDA-MB-231 derivatives were stimulated with 488-Tfn (5 minutes, 10 μg/mL). Cells were fixed on ice, acid-washed and analysed for signal intensity.

FIGS. 8A-I show that SYNJ2 regulates EGFR trafficking and chemotaxis. FIG. 8A—Whole extracts of MDA-MB-231 cells transfected with the indicated siRNAs were immunoblotted as indicated. FIG. 8B—FACS (left) and ¹²⁵I-EGF binding (right; in triplicates) analyses of surface EGFR in the indicated MDA-MB-231 subclones. FIG. 8C—shCtrl and shSYNJ2 cells were grown on fibronectin and immunostained for EGFR and F-actin. Bar, 20 μm. FIG. 8D—Rose plots of tracks of shCtrl and shSYNJ2 MDA-MB-231 cells, which migrated in chemotaxis chambers upon exposure to an EGF gradient. The red tracks indicate cells migrating toward EGF. FIG. 8E—Starved MDA-MB-231 derivatives were treated with EGF (10 ng/mL) and cell lysates were subjected to immunoprecipitation and immunoblotting as indicated. FIG. 8F—Cells were cultured as in C and immunostained for active EGFR (pY1045) and F-actin. Bar, 10 μm. FIG. 8G—The indicated MDA-MB-231 derivatives were treated with EGF (10 ng/ml) for 5 hours and extracts immunoblotted as indicated. FIG. 8H—The indicated MDA-MB-231 derivatives were exposed to Alexa Fluor 488-Tfn (25 μg/ml; 5 min), acid-washed to remove surface-bound ligands, and images taken at the indicated intervals. Normalized fluorescence signals are shown. Bar, 10 μm. FIG. 8I—MDA-MB-231 cells, pre-treated with siCtrl or siSYNJ2, were stimulated with Alexa Fluor 488-EGF (20 μg/ml; 10 min), acid-washed, incubated at 37° C. for the indicated intervals and analysed by FACS.

FIGS. 9A-D show that SYNJ2 is necessary for both vesicular trafficking and focal adhesion formation. FIG. 9A—MDA-MB-231 derivatives (shCtrl and shSYNJ2) were fixed and stained for EEA1, F-actin and nuclei (DAPI). Scale bar, 10 μm. FIG. 9B—MDA-MB-231 derivatives, namely shCtrl and shSYNJ2 cells, were probed for integrin beta-1, F-actin and DAPI (scale bar, 20 μm). FIG. 9C—MDA-MB-231 cells were treated with siCtrl and siSYNJ2 for 48 hours and then immunostained for integrin beta-1 and phosphorylated EGFR. FIG. 9D—Immunofluorescence analysis of MDA-MB-231 derivatives for paxillin, nuclei (DAPI), and F-actin (using TRITC-phalloidin). The paxillin signal was quantified in cytoplasmic regions relative to focal adhesions, and the numbers of focal adhesions per cell were also quantified. In addition, the shapes of focal adhesions were quantified by determining deviations from a perfect circle (eccentricity). Scale bar, 10 μm.

FIGS. 10A-F show that SYNJ2 depletion perturbs phosphoinositide homeostasis, inflates early endosomes and disassembles focal adhesions. FIG. 10A—MDA-MB-231 cells, expressing shCtrl or shSYNJ2, were transfected with a GFP-Rab4 plasmid and 48 hours later cells were fixed and counterstained for F-actin using TRITC-phalloidin. FIG. 10B—MDA-MB-231 derivatives were immunostained for Rab5, F-actin and nuclei (DAPI). Images were quantified for the size and number of Rab5-positive vesicle, as well as for the average cell area. Scale bars, 10 μm. FIG. 10C—Phosphoinositides extracted from ³H-phosphoinositol labeled derivatives of MDA-MB-231 cells, were separated by chromatography and their levels determined in three different experiments (signals normalized to shCtrl cells). FIG. 10D—shCtrl and shSYNJ2 MDA-MB-231 cells were probed for pY1068-EGFR, Paxillin and F-actin (co-localization signal are white). Scale bar, 10 μm. FIG. 10E—shCtrl and shSYNJ2 MDA-MB-231 cells were seeded. Unattached cells were removed 20 min later and attached cells were imaged and quantified for surface area. FIG. 10F—MDA-MB-231 cells, stably expressing shCtrl or shSYNJ2, were plated on RTCA E-plates and real-time impedance measurements were recorded in 5 sec intervals for 80 min, and then in 10 min intervals for additional 80 min. Means of 2 replicates (±S.D.) are shown.

FIGS. 11A-G show that SYNJ2 regulates protease secretion and invadopodium assembly. FIG. 11A—shCtrl and shSYNJ2 MDA-MB-231 cells were cultured in Matrigel for 5 days, fixed and immunostained for MMP-9. Signal intensities were converted into heat-maps and plotted against distance from colony cores. Arrowheads mark spheroid boundaries. Bar, 50 μm. FIG. 11B—Supernatants from control MDA-MB-231 cells and cells stably overexpressing SYNJ2 were analyzed in triplicates for MMP-2 and MMP-9 activity using gelatin zymography. FIG. 11C—MDA-MB-231 cells stably expressing GFP-SYNJ2 were plated onto coverslips pre-coated with cross-linked fluorescent gelatin. Three hours later, cells were probed for GFP and F-actin, and invadopodial structures detected (arrowheads). Bar, 10 μm. FIG. 11D—MDA-MB-231 cells overexpressing SYNJ2 (SYNJ2-OX), as well as cells pre-treated with siCtrl or siSYNJ2 oligonucleotides, were plated on coverslips pre-coated with cross-linked fluorescent gelatin and invadopodial structures were quantified in three independent experiments. FIG. 11E—Invadopodial structures of MDA-MB-231 cells treated with the indicated siRNAs were detected by gelatin degradation, as well as by staining for F-actin or TKS5. Arrowheads (z-axis images) mark invadopodia. Bar, 10 μm. FIG. 11F—MDA-MB-231 cells expressing siCtrl or siSYNJ2 were plated on gelatin-coated coverslips and processed as in C using phalloidin and antibodies to the phosphorylated form of EGFR (tyrosine 1068). Scale bar, 10 μm. FIG. 11G—Media conditioned over 3 days by the indicated MDA-MB-231 derivatives were examined using an ELISA-based assay for EGF-like ligands.

FIGS. 12A-G show that SYJN2 regulates matrix degradation and invadopodia assembly. FIG. 12A—The indicated siRNA-treated MDA-MB-231 cells were plated in triplicates, cultured for 3 days and their conditioned media were separated electrophoretically using a gelatin (0.1%) embedded gel, followed by protein staining to quantify MMP-2 and MMP-9 proteolytic activity. FIG. 12B—Co-immunoprecipitation analysis using GFP-conjugated beads and cleared extracts of MDA-MB-231 cells stably expressing GFP-SYNJ2. FIG. 12C—MDA-MB-231 cells stably expressing GFP-SYNJ2 were transfected with a RFP-Cortactin plasmid and plated on collagen plates. Live-cell image analysis was performed forty-eight hours later, and representative snapshot images of both peripheral and central cell areas were captured. Scale bar 5 μm. FIG. 12D—The indicated derivatives of MDA-MB-231 cells were transfected with a plasmid encoding a Myc-tagged PH domain of Tapp1 (a PI(3,4)P₂ binder) and 48 hours later they were plated on gelatin-coated surfaces. The co-distribution of F-actin, aggregated TKS5 and PI(3,4)P₂ (Tapp1) was visualized and quantified using confocal microscopy. Scale bar, 10 μm FIG. 12E—MDA-MB-231 cells expressing siCtrl or siSYNJ2 were plated onto FITC-gelatin coated glass coverslips and incubated for 3 hours. Cells were then fixed and immunostained for CD44, and counter stained for F-actin with TRITC-phalloidin. Cells were visualized using fluorescence microscopy, and invadopodia were detected by observing holes in the FITC-gelatin matrix. The framed areas are enlarged. Scale bar, 10 μm. FIG. 12F—An antibody to CD44 was used for FACS analysis of surface expression by shCtrl and shSYNJ2 cells. Indicated are the fractions of cells corresponding to the framed regions. FIG. 12G—MDA-MB-231 cells pre-treated with siCtrl or siSYNJ2 were plated onto FITC-gelatin coated glass coverslips and incubated for 3 hours. Cells were then fixed and immunostained for MT1-MMP, and counter stained for F-actin with TRITC-phalloidin. Scale bar, 10 μm.

FIGS. 13A-H show that the enzymatic activity of SYNJ2 propels metastatic spread of mammary tumor cells. FIG. 13A—The indicated derivatives of RFP-expressing MDA-MB-231 cells (2×10⁶/mouse) were implanted in the fat pad of female SCID mice (10-11 per group). Tumor size (mean±S.D.) was measured 2 and 6 weeks post implantation. FIGS. 13B-C—Metastases that appeared six weeks post-implantation in axillary and distant lymph nodes (FIG. 13B), or lungs (FIG. 13C), are shown. Asterisks mark p values: *<0.05, **<0.01 and ***<0.001. FIGS. 13D-F—Control (LacZ) and SYNJ2-overexpressing (SYNJ2-OX) RFP-labelled MDA-MB-231 cells were implanted in animals as in A and tumor size (FIG. 13D), as well as metastases to lymph nodes (FIG. 13E) and lungs (FIG. 13F) were quantified 6 and 8 weeks post implantation. FIGS. 13G-H—The indicated MDA-MB-231-RFP derivatives were injected either intravenously (1.5×10⁵ per mouse; tail vein), or in the mammary fat pad (2.5×10⁶ per mouse) of 5-week old female SCID mice. Four weeks later, lungs from mice injected into the vein were examined for RFP signals (left and middle panels). Peripheral blood was collected from the fat pad-treated group four weeks later. Samples were purified on a gradient of ficoll and the numbers of RFP-positive circulating tumor cells (CTC) were scored per 1×10⁶ FACS readings and normalized to tumor weight.

FIG. 14 is an in vivo imaging of local and distant lymph node metastases. Representative images of local (ipsilateral) and distant (contralateral) lymph node metastases in mice that were inoculated with MDA-MB-231-RFP cells and analysed 6 weeks later (see FIG. 13B). Prior to imaging, mice were anaesthetized and their fur was removed for visualization and quantification of metastases in lymph nodes.

FIG. 15 is a working model depicting the integrated action of SYNJ2 in cell migration and invasion. EGFR-loaded recycling endosomes position active receptors at the ventral membrane, and this is followed by local activation of PI3K. Phosphorylation of membranal PI(4,5)P₂ by PI3K generates PI(3,4,5)P₃, which is dephosphorylated by SYNJ2 to PI(3,4)P₂. The latter recruits TKS5, which anchors Cortactin and nucleates actin polymerization. In parallel, SYNJ2 controls delivery of adhesion molecules like CD44, and proteases like MT1-MMP, to degrade the extracellular matrix (ECM) and establish new invasive structures, the invadopodia. In a similar way, EGFR delivery to the cell periphery leads to breakdown of PI(4,5)P₂ by SYNJ2 (and phospholipase C), which locally activates Dynamin and actin severing enzymes like Cofilin to dissolve cortical actin fibres and initiate actin-filled, integrin-rich protrusions called lamellipodia. The horizontal arrow marks the direction of cell migration. Color-coded segments of the plasma membrane denote specific PI phospholipids.

FIGS. 16A-C show that SYNJ2 is highly expressed in aggressive breast tumors. FIG. 16A—Immunohistochemistry and tissue microarrays were used to stratify 331 invasive breast carcinomas according to SYNJ2 abundance (high, medium and low). The relative fraction of tumors is presented according to clinical subtypes. FIG. 16B—Representative images of SYNJ2 staining demonstrating intensities and patterns (magnified in the right column) observed in a luminal case (an asterisk marks expression by endothelial cells as control), and both basal-like and HER2-overexpressing breast tumors. FIG. 16C—Kaplan-Meier curves stratified according to SYNJ2 mRNA expression in cohorts of 286 (left; GSE2034) or 99 (right; GSE19783) breast cancer patients.

FIGS. 17A-B present a scheme (FIG. 17A) depicting an exemplary assay for detecting binding of a molecule (black circle) labeled by a fluorescent probe (white circle) to a large molecule (ellipse), wherein binding results in an increase in fluorescence polarization following excitation by polarized light, and a bar graph (FIG. 17B) showing the increase of polarization fluorescently-labeled phosphatidyl inositol (3,4)-biphosphate (PIP2; probe) in the presence of a protein (detector) which binds PIP2, and the increase in the presence of unlabeled PIP2 or a combination of SYNJ2 and phosphatidyl inositol triphosphate (PIP3).

FIG. 18 depicts the amino acid and nucleic acid sequences of the Flag-TAPP1 PH domain-His that was cloned into pET28 plasmid and expressed in E. coli. The first TAPP1-PH domain is marked in yellow.

FIG. 19 presents images of MDA-MB-231 cell spheroids seeded in 5% Matrigel, 0 or 96 hours after being treated with 2 μM pyrvinium pamoate (Compound 18) or carrier (DMSO).

FIGS. 20A-B present a plot (FIG. 20A) and images (FIG. 20B) showing migrating MCF10A cells in the presence of various concentrations (in the range of 0.125-10 μM) pyrvinium pamoate (Compound 18) or carrier (DMSO) (FIG. 20A shows mean±standard deviation, and distribution of experimental values, with p values for differences between 1 an 2 μM pyrvinium pamoate and DMSO control; FIG. 20B shows representative results from a corresponding experiment).

FIG. 21 is a box plot showing the tumor mass of MDA-MB-231 breast cancer cells in mice 6 weeks after beginning to inject the mice twice per week with the exemplary compound NP-360 (5 mg/kg; Compound 12) or carrier (DMSO); vertical lines (whiskers) indicate range of mean±standard deviation, horizontal lines indicate median, and boxes indicate 25% to 75% percentiles (p=0.0372; injection in each mouse began when tumor volume was approximately 3×3×3 mm³).

FIG. 22 is a plot showing the tumor mass of MDA-MB-231 breast cancer cells in mice 6 weeks after beginning to inject the mice twice per week with the exemplary compound NP-3195 (Compound 2) or carrier (DMSO) (mean±standard deviation, and distribution of experimental values; injection in each mouse began when tumor volume was approximately 3×3×3 mm³).

FIGS. 23A-B present images (FIG. 23A) and a plot (FIG. 23B) showing metastases that appeared in the lungs six weeks post-implantation of MDA-MB-231 breast cancer cells in mice treated by injection of the exemplary compound NP-3195 (Compound 2) or carrier (DMSO) (FIG. 23A shows representative samples; FIG. 23B shows mean±standard deviation, and distribution of experimental values).

FIG. 24 presents images of fluorescent-labeled EGFR (endothelial growth factor receptor) in MDA-MB-231 cells subjected to knockdown of SYNJ2 (shSYNJ2) or control knockdown (shCtrl) (upper row) and in naive cells (bottom row) treated with 0.78 μM pyrvinium pamoate (Compound 18) or with carrier (DMSO) (blue staining of nuclei with DAPI is used for contrast).

### DESCRIPTION OF SPECIFIC EMBODIMENTS OF THE INVENTION

The present invention, in some embodiments thereof, relates to cancer therapy and more particularly, but not exclusively, to compounds, compositions and methods for preventing tumor metastasis, and for treating cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Growth factors propel cell migration and metastasis, but underlying mechanisms are incompletely understood.

The present inventors have now identified synaptojanin-2 (SYNJ2) as a master module in regulating invadopodia and lamellipodia in vitro and cancer metastasis in vivo.

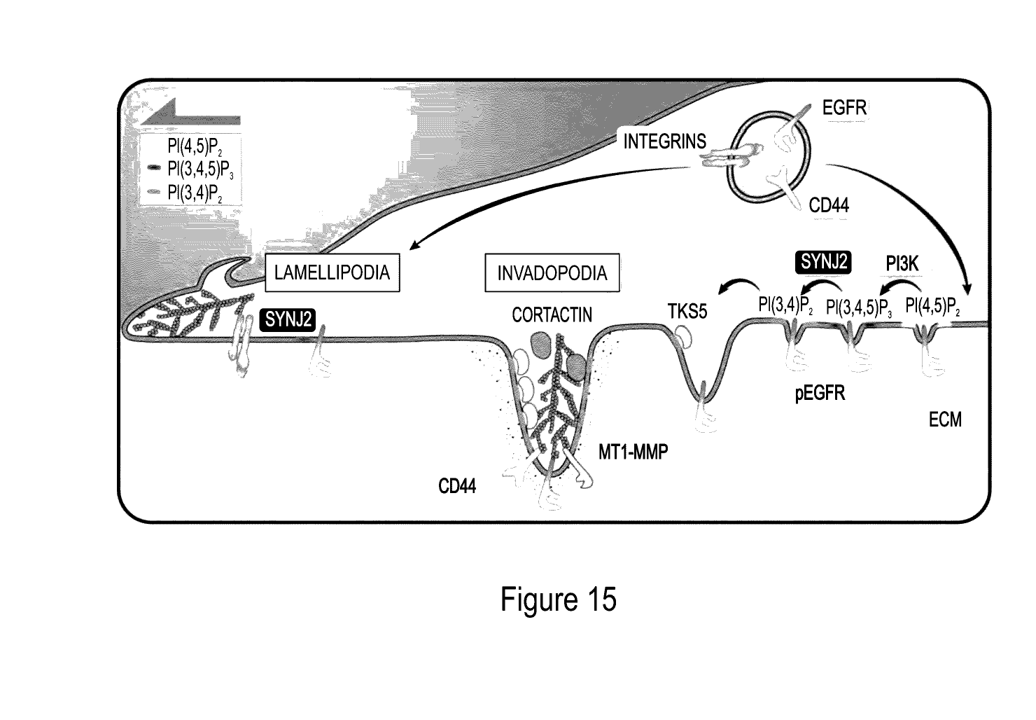

As is illustrated herein below and in the Examples section which follows, the present inventors substantiated their finding in vitro, in animals and in patient specimens. Specifically, employing EGF-stimulated mammary cells the present inventors link the lipid phosphatase synaptojanin 2 (SYNJ2) to an invasive phenotype, and relate high SYNJ2 to short survival rates of cancer patients. Knockdown of SYNJ2 robustly impaired metastasis of mammary tumor cells in an animal model. In vitro, SYNJ2-depleted cells exhibited derailed trafficking of EGFR and integrins, resulting in deformed focal adhesions, arrested lamellipodia and disappearance of invadopodia. Without being bound to theory it is suggested that recycling of active EGFRs focally promotes SYNJ2-mediated dephosphorylation of specific phosphoinositol lipids, thereby instigating formation of both invadopodia and lamellipodia and facilitates tumor progression (see FIG. 15).

The present inventors have further screened a variety of small molecules and uncovered a group of small molecule which act as inhibitors of SYNJ2. The uncovered group of small molecules was found to possess common structural features which provide SYNJ2 inhibitory activity. Accordingly, small molecules which may be used to prevent tumor metastasis as described herein are disclosed.

Reference is made to Table 1, which depicts chemical structures and activity of molecules which selectively inhibit SYNJ2 enzyme activity.

As shown in Table 1, compounds which selectively inhibit SYNJ2 enzyme activity can be characterized as comprising one or two hydroxylated phenyl groups attached (optionally via a short linking moiety) to one another or to a core moiety which comprises a benzopyran derivative and/or a saccharide or saccharide-like moiety such as shikimate or quinate.

Other compounds in Table 1 are glucosides such as glucosides of terpenoids.

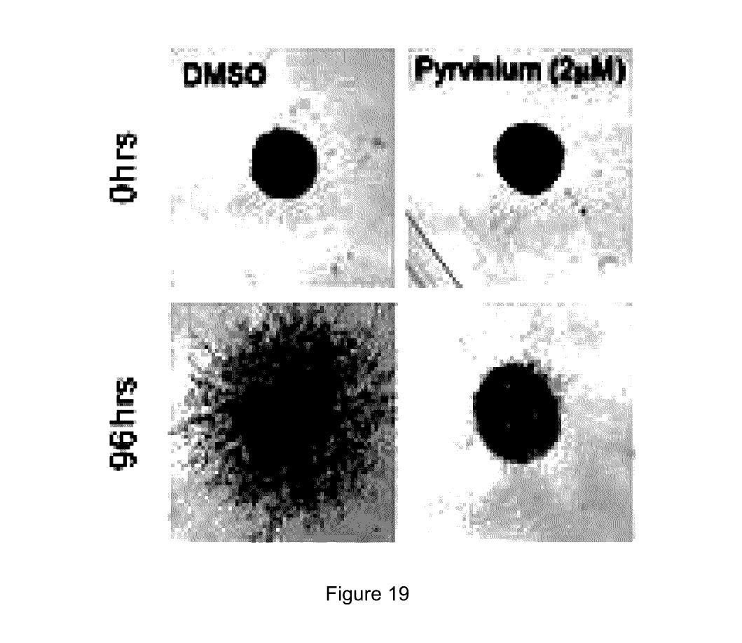

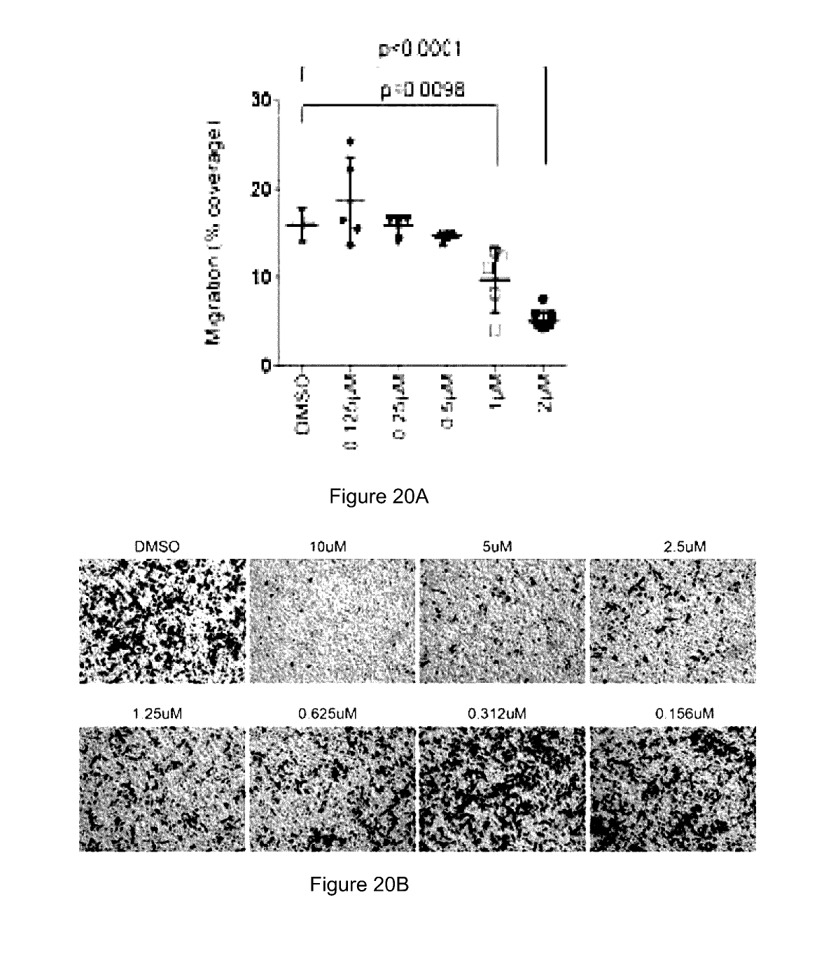

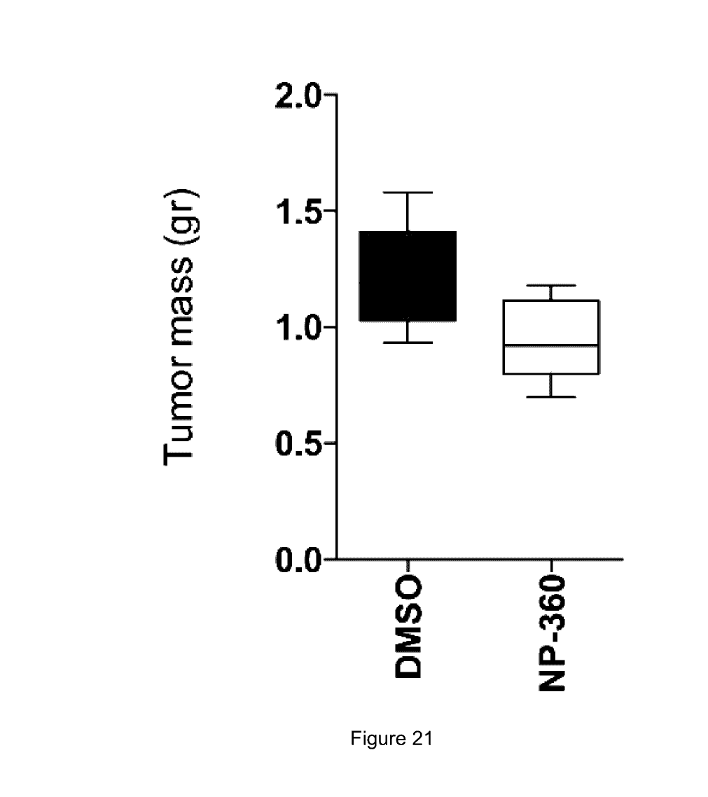

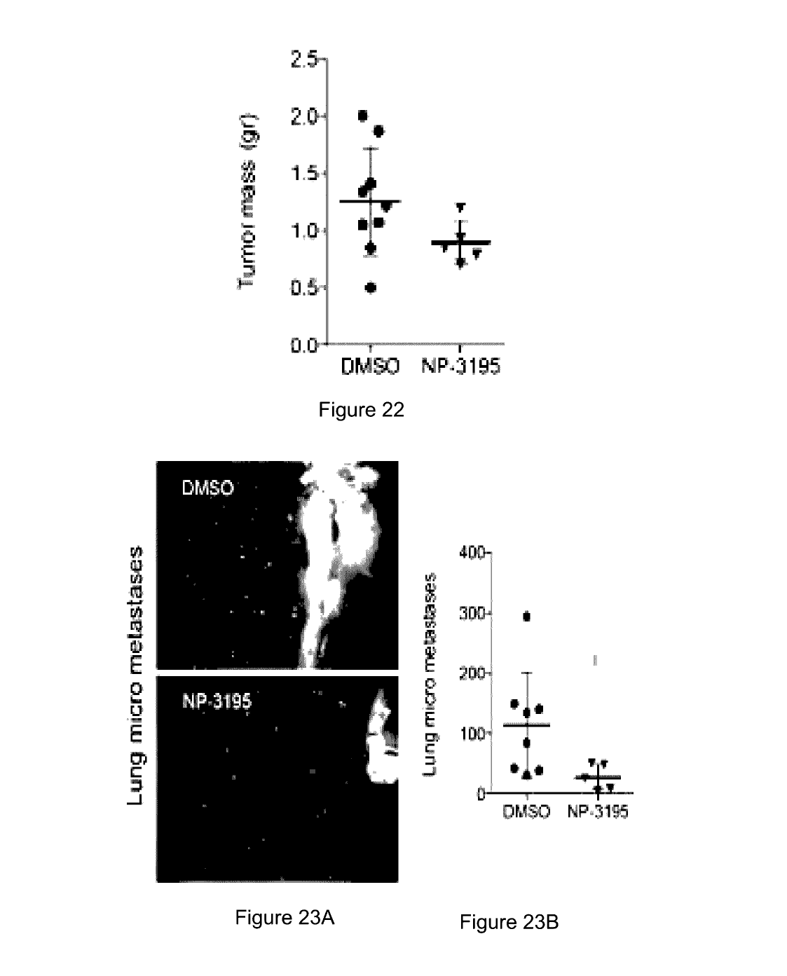

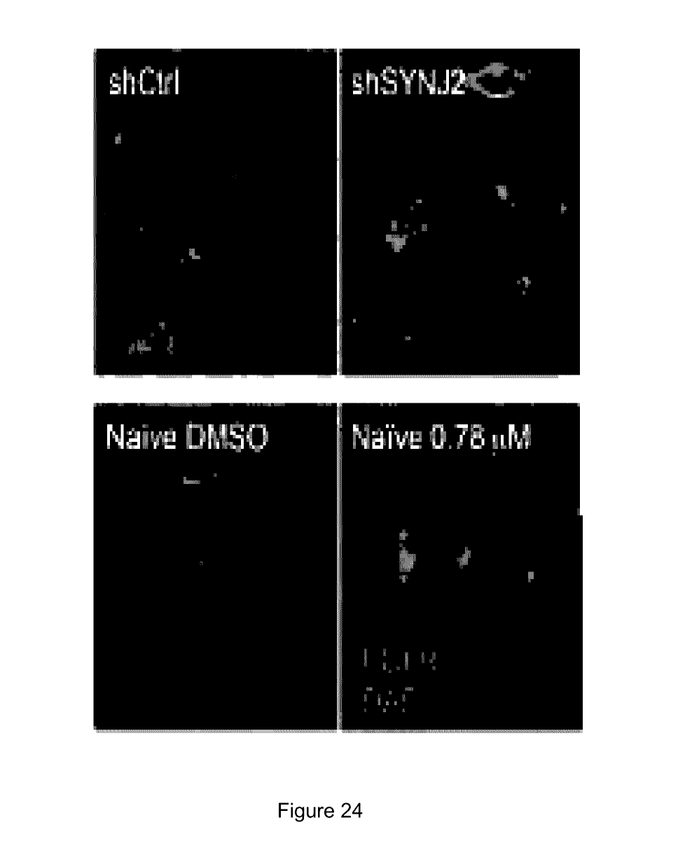

Reference is further made to FIGS. 19-24, which show that exemplary SYNJ2 inhibitors depicted in Table 1 (Compound 18 in FIGS. 19-20B, Compound 12 in FIG. 21, and Compound 2 in FIGS. 22-23B) inhibit cancer cell invasion (FIG. 19) and migration (FIGS. 20A-20B) in vitro, and tumor growth (FIGS. 21-22) and metastasis (FIGS. 23A-23B) in mice. FIG. 24 further shows that Compound 18 causes redistribution of endothelial growth factor to within the cell.

Without being bound by any particular theory, it is believed that hydroxylated phenyl groups and saccharides and saccharide-like moieties effect selective SYNJ2 inhibition by being similar in structure to inositol derivatives which are the natural substrates of SYNJ2, in that they comprise a six-membered ring substituted by hydroxy groups.

Thus, according to an aspect of some embodiments of the invention there is provided a method of preventing tumor metastasis, the method comprising administering to a subject in need thereof a therapeutically effective amount of an inhibitor of synaptojanin 2 (SYNJ2) as described herein, thereby preventing tumor metastasis.

According to another aspect of some embodiments of the present invention, there is provided a compound described herein as a SYNJ2 inhibitor, for use in preventing tumor metastasis.

According to another aspect of some embodiments of the present invention, there is provided a use of a compound described herein as a SYNJ2 inhibitor, in the manufacture of a medicament for preventing tumor metastasis.

In some embodiments of any of the embodiments described herein relating to tumor metastasis, the compound is Compound 2 (NP-3195).

As used herein the phrase “tumor metastasis” refers to a malignant tumor spreading out of its primary location to other parts of the body, e.g., breast cancer which metastasizes to the lungs. Tumor metastasis often involves migration of tumor cells.

As used herein the terms “cancer” and “tumor” are interchangeably used. The term refers to a malignant growth or tumor caused by abnormal and uncontrolled cell proliferation (cell division).

As used herein the term “preventing” refers to arresting, halting, inhibiting the metastatic process or progression and subsequent metastasis.

According to another aspect of some embodiments of the present invention, there is provided a method of treating cancer the method comprising, administering to a subject in need thereof a therapeutically effective amount of an inhibitor of synaptojanin 2 (SYNJ2) described herein and an inhibitor of a cell surface receptor associated with an onset or progression of cancer, thereby treating cancer.

According to another aspect, there is provided a compound described herein as a SYNJ2 inhibitor, and an inhibitor of a cell surface receptor associated with an onset or progression of cancer, for use in treating cancer.

According to another aspect, there is provided a use of a compound described herein as a SYNJ2 inhibitor, and an inhibitor of a cell surface receptor associated with an onset or progression of cancer, in the manufacture of a medicament for treating cancer.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein the term “subject” refers to a mammal (e.g., human), for example, one who has been diagnosed with cancer.

As used herein synaptojanin-2 or SYNJ2 refers to synaptic inositol-1,4,5-trisphosphate 5-phosphatase 2, EC 3.1.3.36, a ubiquitously expressed inositol polyphosphate 5-phosphatase.

As used herein the phrases “inhibitor of synaptojanin-2”, “synaptohanin-2 inhibitor” and variations thereof refer to a molecule which decreases activity of SYNJ2.

In some embodiments of any of the aspects described herein, the compound which is a SYNJ2 inhibitor has the general formula I:

X-L-[(Y)i-(Z)j]-(L-X)k Formula I

or pharmaceutically acceptable salts thereof, wherein:

i, j and k are each independently 0 or 1, wherein at least one of i, j and k is 1;

L is absent or is a linking moiety (e.g., from 1 to 3 atoms in length);

X is an aryl group substituted by one or more hydroxy group and/or one or more analog of a hydroxy group selected from the group consisting of thiohydroxy, alkoxy, aryloxy, thioalkoxy and thioaryloxy;

Z is selected from the group consisting of a monosaccharide moiety, a disaccharide moiety, a shikimate moiety and a quinate moiety; and

Y is a bicyclic moiety having the general formula II:

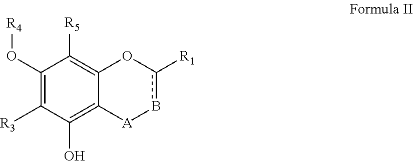

wherein:

A is absent or is CH₂, C═O, C═S or C═NR₆;

B is absent or is O, S, NR₇, CH, CH₂, C—O—R₂, C—S—R₂, C—N(R₈)—R₂, CH—O—R₂, CH—S—R₂ or CH—N(R₉)—R₂;

R₁-R₅ are each independently selected from the group consisting of hydrogen, methyl, aryl (e.g., phenyl) and a covalent bond with an L, Z or X moiety as described herein, if present;

R₆-R₉ are each independently selected from the group consisting of hydrogen and alkyl (e.g., C₁₋₄ alkyl); and

the dashed line denotes a saturated or unsaturated bond, wherein when the dashed line denotes a saturated bond, B is O, S, NR₇, CH₂, CH—S—R₂, CH—N(R₉)—R₂ or CH—O—R₂, and when the dashed line denotes an unsaturated bond, B is CH, C—S—R₂, C—N(R₈)—R₂ or C—O—R₂.

In embodiments wherein k is 1, the compound comprises two X moieties which may be the same or different.

Herein, any optional embodiment described herein for any one or more of the variables X, Y, X, L, i, j, k, A, B, D, E and R₁-R₅ is intended to be combined in any possible combination with any of the optional embodiments for the remaining variables described herein, unless explicitly indicated otherwise.

In some embodiments, A is CH₂ or C═O.

In some embodiments, B is CH, CH₂, C—O—R₂, or CH—O—R₂.

In some embodiments, the linking moiety is selected from the group consisting of C(═O), C(═S), C(═NR₁₀) and a saturated or unsaturated alkylene chain, preferably from 1 to 3 atoms in length, and optionally interrupted by C(═O), C(═S) and/or C(═NR₁₀), wherein R₁₀ is defined as R₆-R₉ are defined herein.

Examples of suitable linking moieties include, but are not limited to, C(═O), CH₂C(═O), CH₂CH₂C(═O), CH₂C(═O)CH₂, CH═CH, CH═CH—CH₂, CH═CH—C(═O), and an alkylene chain (e.g., CH₂ or CH₂CH₂CH₂). Optionally, the linking moiety is a bond.

In exemplary embodiments, L is absent or is a linking moiety selected from the group consisting of C(═O), CH═CH, CH═CH—C(═O) and CH₂.

In some embodiments, the aryl is a phenyl.

In some embodiments, the aryl is a hydroxylated aryl. In some embodiments, the aryl is a hydroxylated phenyl.

Herein, the terms “hydroxylated aryl” and “hydroxylated phenyl” refer to an aryl group or a phenyl group, respectively, substituted by any number of hydroxy groups (and optionally also one or more alkoxy or aryloxy groups, preferably methoxy groups), whereas the term “hydroxyphenyl” refers to a phenyl group substituted by one hydroxy group, and is also referred to as phenol.

In some embodiments, X is a hydroxylated phenyl group selected from the group consisting of trihydroxyphenyl (dihydroxyphenol), dihydroxyphenyl (hydroxyphenol), hydroxyphenyl (phenol), methoxydihydroxyphenyl, and methoxyhydroxyphenyl;

According to any one of the embodiments described herein, and any combination thereof, in some embodiments, X is selected from the group consisting of 3,4,5-trihydroxyphenyl (an exemplary trihydroxyphenyl); 3,4-dihydroxyphenyl, 3,5-dihydroxyphenyl, 3,5-dihydroxyphenyl and 2,3-dihydroxyphenyl (exemplary dihydroxyphenyls); 6-methoxy-2,4-dihydroxyphenyl (an exemplary methoxydihydroxyphenyl); 3-methoxy-4-hydroxyphenyl and 3-hydroxy-4-methoxyphenyl (exemplary methoxyhydroxyphenyls); and 4-hydroxyphenyl (an exemplary hydroxyphenyl).

According to any one of the embodiments described herein, and any combination thereof, in some embodiments, X comprises at least two hydroxy or methoxy groups. In some embodiments, X comprises at least two hydroxy groups. In some embodiments, X comprises at least three hydroxy groups, for example, wherein X is trihydroxyphenyl.

Without being bound by any particular theory, it is believed that the number of hydroxy groups (at least up to 3 hydroxy groups) is associated with SYNJ2 inhibition. Thus, as exemplified herein in Table 1 in the Examples section, addition of a hydroxy group to a phenyl moiety of catechin-7-gallate, resulting in gallocatechin-7-gallate, decreased the IC50 of the compound more than 3-fold.

In some embodiments, R₁ is selected from the group consisting of methyl, phenyl and a covalent bond. In some embodiments, R₁ is a covalent bond.

In exemplary embodiments, R₁ is a bond with an X, that is, an L at this position is absent, or alternatively, R₁ is phenyl. In such embodiments, a substituted or non-substituted phenyl is attached to the Y moiety at the R₁ position.

Moieties and compounds in which a substituted or non-substituted phenyl is attached to the Y moiety at the R₁ position are referred to herein as “flavonoid” moieties and compounds.

In some embodiments, R₂ is selected from the group consisting of hydrogen and a covalent bond. In exemplary embodiments, R₂ is a bond to a 3,4-dihydroxyphenyl moiety.

In some embodiments, when B is C—O—R₂, R₂ is not hydrogen. In some embodiments, when B is C—O—R₂, R₂ is a bond with a Z. In some embodiments, the Z is a disaccharide moiety (e.g., mannosyl galactose) or a monosaccharide attached to -L-X (e.g., a galloyl-substituted rhamnosyl).

In some embodiments, R₃ is selected from the group consisting of hydrogen, methyl and a covalent bond. In exemplary embodiments, when R₃ is a covalent bond, the bond is with an L linking moiety which is CH₂.

In some embodiments, R₄ is selected from the group consisting of hydrogen and a covalent bond.

In some embodiments, R₄ is a covalent bond with X or L, and X is 3,4,5-trihydroxyphenyl.

In some embodiments, R₄ is a covalent bond with an L linking moiety which is C═O. In exemplary embodiments, X is 3,4,5-trihydroxyphenyl and L is C═O, such that R₄ is a bond to a galloyl (3,4,5-trihydroxybenzoyl) group.

In some embodiments, R₄ is a covalent bond with Z (e.g., when Y is part of a flavonoid moiety). In some embodiments, Z is a monosaccharide (e.g., glucosyl). In some embodiments, the monosaccharide is attached at the 1-position thereof. In exemplary embodiments, Z is 1-glucosyl (e.g., 1-β-glucosyl).

In some embodiments, R₅ is selected from the group consisting of hydrogen and a covalent bond. In some embodiments, R₅ is hydrogen.

In some embodiments, when R₅ is a covalent bond, the bond is with L, and L is CH₂.

In some embodiments, when the dashed line denotes a saturated bond, such that at least one of the two carbon atoms connected by the dashed line is chiral.

In some such embodiments, the carbon atom attached to R₁ is chiral and is in an (S) configuration.

In some such embodiments, the carbon atom of B is chiral (e.g., when B is CH—O—R₂), and is an (S) configuration when A is C═O, and in an (R) configuration when A is CH₂. It is to be appreciated that the aforementioned configurations of B represent essentially the same configuration, but that the identity of A affects the notation of the configuration.

In some embodiments, the carbon atom attached to R₁ is in an (S) configuration, and the carbon atom of B is in an (S) configuration when A is C═O, and in an (R) configuration when A is CH₂.

In some embodiments wherein B is CH₂ (and therefore not chiral), the carbon atom attached to R₁ is chiral and is in an (R) configuration.

The Z moiety described herein has a structure of a molecule (e.g., a monosaccharide, disaccharide, shikimate or quinate molecule) in which a hydroxy group is replaced with a Y moiety as described herein, and/or a hydrogen atom (e.g., a hydrogen atom of a hydroxy group) is replaced by an L or X moiety as described herein.

According to any one of the embodiments described herein, and any combination thereof, in some embodiments, the monosaccharide is a hexose.

According to any one of the embodiments described herein, and any combination thereof, in some embodiments, the disaccharide comprises two hexose moieties.

According to any one of the embodiments described herein, and any combination thereof, in some embodiments, the hexose is a D-hexose. Exemplary D-hexoses include D-glucose, D-mannose and D-galactose.

According to any one of the embodiments described herein, and any combination thereof, in some embodiments, at least a portion of the monosaccharide or disaccharide comprises a pyranose, that is, at least a portion in solution form a six-membered ring comprising 5 carbon atoms and one oxygen atom. The pyranose form may be in equilibrium with a non-pyranose form (e.g., an aldehyde form) of the saccharide. In some embodiments, the disaccharide comprises two pyranose moieties.

According to any one of the embodiments described herein, and any combination thereof, in some embodiments, a Z moiety which is a saccharide moiety is attached (e.g., to Y, L or X) via a glycosidic bond.

Herein, the phrase “glycosidic bond” refers to a bond at a position of the saccharide which comprises a hemiacetal (derived from an aldehyde) or hemiketal (derived from a ketone) group in the unbound saccharide molecule, for example, the 1-position in glucose, mannose, and galactose.

Without being by any particular theory, it is believed that a glycosidic bond stabilizes a cyclic (e.g., pyranose) form of a monosaccharide, which comprises a six-membered ring substituted by multiple hydroxy groups, which is associated with SYNJ2 inhibition.

Examples of suitable monosaccharides include glucose (e.g., D-glucose), mannose (e.g., L-mannose, D-mannose), galactose (e.g., D-galactose) and rhamnose (e.g., L-rhamnose). In exemplary embodiments, the monosaccharide is a glucose or rhamnose moiety, e.g., attached via a glycosidic bond. In exemplary embodiments, the disaccharide is mannose (e.g., L-mannose) moiety attached to a galactose (e.g., D-galactose) moiety, e.g., wherein the mannose is attached via a mannose glycosidic bond and the galactose moiety is attached to a Y, L or X moiety described herein via a galactose glycosidic bond.

In embodiments wherein a saccharide moiety is substituted at two positions (e.g., by two moieties as described herein), one of the positions is the 6-position of the saccharide (e.g., wherein the other position is a glycosidic bond as described herein).

Without being bound by any particular theory, it is believed that D-glucose is a particularly suitable monosaccharide, as it typically assumes a configuration wherein the free hydroxy groups are attached to a pyranose ring at equatorial positions (rather than axial positions), which renders the hydroxy groups more accessible to the environment, and accords with the structure of phosphatidyl inositol, in which almost all of the hydroxy groups are in an equatorial position.

Herein, the terms “shikimate” and “shikimic acid” each refer to 3,4,5-trihydroxycyclohex-1-ene-1-carboxylic acid and to pharmaceutically acceptable salts thereof, including any stereoisomer thereof. In exemplary embodiments, the terms refer to the (3R,4S,5R) stereoisomer.

Herein, the terms “quinate” and “quinic acid” each refer to 1,3,4,5-tetrahydroxycyclohexanecarboxylic acid and to pharmaceutically acceptable salts thereof, including any stereoisomer thereof. In some embodiments, the terms refer to a (3R,4S,5R) stereoisomer. In exemplary embodiments, the terms refer to the (1S,3R,4S,5R) stereoisomer.

It is to be appreciated that shikimate and quinate are similar in structure to pyranose monosaccharides, in that they comprise a six-membered ring substituted by multiple hydroxy groups.

According to any one of the embodiments described herein, and any combination thereof, in some embodiments, Z comprises a single ring, that is, Z is a monosaccharide, shikimate or quinate.

According to any one of the embodiments described herein, and any combination thereof, in some embodiments of any of the aspects described herein, i and j are 0, and k is 1. In exemplary embodiments, L is not absent, such that the two X moieties are linked by a linking moiety. In exemplary embodiments, the X moieties are different from one another.

According to any one of the embodiments described herein, and any combination thereof, in some embodiments, each X is independently selected from the group consisting of 2,4-dihydroxyphenyl, 3,4-dihydroxyphenyl, 3,5-dihydroxyphenyl and 6-methoxy-2,4-dihydroxyphenyl.

According to any one of the embodiments described herein, and any combination thereof, in some embodiments, L is selected from the group consisting of C(═O) and CH═CH.

According to any one of the embodiments described herein, and any combination thereof, in some embodiments, L is C(═O) and each X is selected from the group consisting of 2,4-dihydroxyphenyl, 3,4-dihydroxyphenyl, and 6-methoxy-2,4-dihydroxyphenyl. In exemplary embodiments, each X is 3,4-dihydroxyphenyl and 6-methoxy-2,4-dihydroxyphenyl.

According to any one of the embodiments described herein, and any combination thereof, in some embodiments, L is CH═CH, one X is 3,5-dihydroxyphenyl, and the other X is selected from the group consisting of 2,4-dihydroxyphenyl, 3,4-dihydroxyphenyl, and 6-methoxy-2,4-dihydroxyphenyl. In exemplary embodiments, one X is 3,5-dihydroxyphenyl and the other X is 2,4-dihydroxyphenyl.

According to any one of the embodiments described herein, and any combination thereof, in some embodiments of any of the aspects described herein, i is 1 and j is 0, such that the compound has the general formula:

X-L-Y(-L-X)k

According to any one of the embodiments described herein, and any combination thereof, in some embodiments, the dashed line in Y denotes a saturated bond (e.g., wherein the carbon atoms connected by the dashed line exhibit stereochemistry as described herein).

According to any one of the embodiments described herein, and any combination thereof, in some embodiments, each X is independently selected from the group consisting of 3,4,5-trihydroxyphenyl, 3,4-dihydroxyphenyl, 2,3-dihydroxyphenyl and 4-hydroxyphenyl.

According to any one of the embodiments described herein, and any combination thereof, in some embodiments, i is 1, j is 0, and k is 1, such that the compound has the general formula:

X-L-Y-L-X

According to any one of the embodiments described herein, and any combination thereof, in some embodiments, each X is independently selected from the group consisting of 3,4,5-trihydroxyphenyl, 3,4-dihydroxyphenyl and 2,3-dihydroxyphenyl.

According to any one of the embodiments described herein, and any combination thereof, in some embodiments, each L is independently absent or is C(═O) or CH₂.

According to any one of the embodiments described herein, and any combination thereof, in some embodiments, an L which is C(═O) is attached to an X which is 3,4,5-trihydroxyphenyl, such that -L-X is galloyl (3,4,5-trihydroxybenzoyl).

According to any one of the embodiments described herein, and any combination thereof, in some embodiments, an L which is CH₂ is attached to an X which is 2,3-dihydroxyphenyl, such that -L-X is 2,3-dihydroxybenzyl.

According to any one of the embodiments described herein, and any combination thereof, in some embodiments, R₂ and R₄ are covalent bonds. In some embodiments, R₂ is a bond to a 3,4-dihydroxyphenyl moiety, and R₄ is a bond to a galloyl moiety.

According to any one of the embodiments described herein, and any combination thereof, in some embodiments, R₁ and R₂ are covalent bonds. In some embodiments, R₁ is a bond to a 3,4-dihydroxyphenyl or 3-methoxy-4-hydroxyphenyl moiety, and R₂ is a bond to a galloyl, disaccharide or galloyl-substituted monosaccharide moiety.

According to any one of the embodiments described herein, and any combination thereof, in some embodiments, R₁ and R₄ are covalent bonds. In some embodiments, R₁ is a bond to a 3,4-dihydroxy or 3,4,5-trihydroxyphenyl moiety, and R₄ is a bond to a galloyl or monosaccharide (e.g., glucosyl) moiety.

According to any one of the embodiments described herein, and any combination thereof, in some embodiments, R₃ and R₅ are covalent bonds. In some embodiments, each of R₃ and R₅ is a bond with an L that is CH₂ (e.g., a bond with 2,3-dihydroxybenzyl). In exemplary embodiments wherein R₃ and R₅ are covalent bonds, R₁ is phenyl.

According to any one of the embodiments described herein, and any combination thereof, in some embodiments, i is 1, j is 0, and k is 0, such that the compound has the general formula:

X-L-Y

According to any one of the embodiments described herein, and any combination thereof, in some embodiments, each X is independently selected from the group consisting of 3,4,5-trihydroxyphenyl, 3,4-dihydroxyphenyl and 4-hydroxyphenyl.

According to any one of the embodiments described herein, and any combination thereof, in some embodiments, L is absent. In exemplary embodiments, X is attached at the R₁ position of Y, such that the compound is a flavonoid.

According to any one of the embodiments described herein, and any combination thereof, in some embodiments, any chiral carbon atom attached to the dashed line in general formula II is in an (S) configuration.

According to any one of the embodiments described herein, and any combination thereof, in exemplary embodiments, B is CHOH.

According to any one of the embodiments described herein, and any combination thereof, in some embodiments, R₃ is hydrogen or methyl, and R₄ is hydrogen.

According to any one of the embodiments described herein, and any combination thereof, in some embodiments of any of the aspects described herein, i is 0 and j is 1, such that the compound has the general formula:

X-L-Z(-L-X)k

According to any one of the embodiments described herein, and any combination thereof, in some embodiments, X is selected from the group consisting of 3,4,5-trihydroxyphenyl, 3,4-dihydroxyphenyl and 3-hydroxy-4-methoxyphenyl.

According to any one of the embodiments described herein, and any combination thereof, in some embodiments, L is C(═O) or CH═CH—C(═O), for example, such that the L is attached to the Z by an ester bond.

According to any one of the embodiments described herein, and any combination thereof, in some embodiments, Z is a monosaccharide (e.g., a hexose), shikimate or quinate. In some embodiments, the monosaccharide is in a pyranose configuration. Glucose (e.g., D-glucopyranose) is an exemplary monosaccharide.

According to any one of the embodiments described herein, and any combination thereof, in some embodiments wherein Z comprises a monosaccharide (e.g., wherein Z is a monosaccharide), the monosaccharide is attached to an L and/or X via an oxygen atom at a 1-position and/or 6-position of the monosaccharide.

According to any one of the embodiments described herein, and any combination thereof, in some embodiments wherein Z is shikimate or quinate, the shikimate or quinate is attached to an L and/or X via an oxygen atom at a 3-position and/or 5-position of the shikimate or quinate.

According to any one of the embodiments described herein, and any combination thereof, in some embodiments, i is 0, j is 1, and k is 1, such that the compound has the general formula:

X-L-Z-L-X

According to any one of the embodiments described herein, and any combination thereof, in some embodiments, the L moieties in the above formula are different from one another.

In exemplary embodiments, the L moieties are the same.

According to any one of the embodiments described herein, and any combination thereof, in some embodiments, the X moieties in the above formula are different from one another. In some embodiments, the X moieties are different, and the L moieties are different. In exemplary embodiments, the X moieties are different, and the L moieties are the same.

According to any one of the embodiments described herein, and any combination thereof, in some embodiments, the X moieties are the same. In some embodiments, the X moieties are the same, and the L moieties are different. In exemplary embodiments, the X moieties are the same, and the L moieties are the same.

According to any one of the embodiments described herein, and any combination thereof, in some embodiments, an L which is C(═O) is attached to an X which is 3,4,5-trihydroxyphenyl, such that -L-X is galloyl (3,4,5-trihydroxybenzoyl).

According to any one of the embodiments described herein, and any combination thereof, in some embodiments, an L which is CH═CH—C(═O) is attached to an X which is a dihydroxyphenyl or methoxyhydroxyphenyl (e.g., 3,4-dihydroxyphenyl or 3-hydroxy-4-methoxyphenyl), such that -L-X is dihydroxycinnamoyl or an O-methylated derivative thereof.

According to any one of the embodiments described herein, and any combination thereof, in some embodiments wherein Z comprises a monosaccharide (e.g., wherein Z is a monosaccharide), the monosaccharide is attached to an L and/or X via oxygen atoms at the 1-position and 6-position of the monosaccharide.

According to any one of the embodiments described herein, and any combination thereof, in some embodiments wherein Z is shikimate or quinate, the shikimate or quinate is attached to an L and/or X via oxygen atoms at a 3-position and 5-position of the shikimate or quinate.

According to any one of the embodiments described herein, and any combination thereof, in some embodiments of any of the aspects described herein, i is 1 and j is 1, such that the compound has the general formula:

X-L-[Y-Z]-(L-X)k

It is to be understood that each -L-X can independently be bound to the Y moiety or Z moiety in the Y-Z core.

According to any one of the embodiments described herein, and any combination thereof, in some embodiments, at least one -L-X is bound to the Y moiety. In some embodiments, one -L-X is bound to the Y moiety, such that the compound has the general formula:

X-L-Y-Z-(L-X)k

According to any one of the embodiments described herein, and any combination thereof, in some such embodiments, an X moiety is attached to the Y moiety at the R₁ position, such that the compound comprises a flavonoid moiety attached to the Z moiety.

According to any one of the embodiments described herein, and any combination thereof, in some embodiments, Z is 1-glucosyl (e.g., attached to the R₂ or R₄ position of Y). In some embodiments, the compound comprises a flavonoid moiety and k is 0, such that the compound is a flavonoid glucoside.

According to any one of the embodiments described herein, and any combination thereof, in some embodiments, Z is substituted or non-substituted 1-rhamnosyl (e.g., attached at the R₂ position of Y). In some embodiments, the 1-rhamnosyl is substituted at the 2-position thereof. In some embodiments, k is 1, and the 1-rhamnosyl is substituted by -L-X (as described herein). In exemplary embodiments, the 1-rhamnosyl is substituted at the 2-position by galloyl.

According to any one of the embodiments described herein, and any combination thereof, in some embodiments, i, j and k are each 1, such that the compound comprises at least 4 cyclic moieties, namely, 2 X moieties described herein, a Y moiety and a Z moiety described herein.

According to any one of the embodiments described herein, and any combination thereof, in some embodiments (e.g., when i, j and k are each 1), a carbon atom in the Z moiety which is attached to the Y moiety is adjacent to a carbon atom in the Z moiety which is attached to -L-X, for example, wherein a 1-position of Z is attached to Y and a 2-position is attached to -L-X. Without being bound by any particular theory, it is believed that such a configuration is more compact, allowing for a shorter distance between the -L-X and Y, and therefore relatively similar in structure (and activity) to compounds wherein Y is attached directly to at least one -L-X (e.g., as described herein).

According to any one of the embodiments described herein, and any combination thereof, in some embodiments, at least one of i, j and k is 0, such that the compound comprises a total of no more than 3 X, Y and Z moieties. In some embodiments, the sum of i, j and k is 2, such that the compound comprises a total of 3 X, Y and Z moieties. In some embodiments, a compound wherein the sum of i, j and k is 2 is a flavonoid attached to Z (e.g., a monosaccharide) or -L-X at the R₄ position (with an X moiety also being present at the R₁ position, as described herein).

Without being bound by any particular theory, it is noted that both compounds in Table I which have the general formula I and which exhibit an IC50 of less than 2 μM (i.e., Compound 10 (gallocatechin-7-gallate) and Compound 15 (pyracanthoside)) are flavonoids attached to Z or -L-X at the R₄ position, and it is therefore believed that such a structure is a particularly active form of general formula I.

In some embodiments of any of the aspects described herein, the compound described herein as an SNJ2 inhibitor has the general formula III:

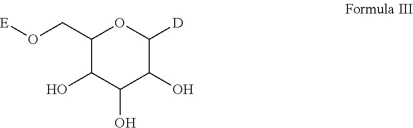

or a pharmaceutically acceptable salt thereof, wherein:

D is selected from the group consisting of:

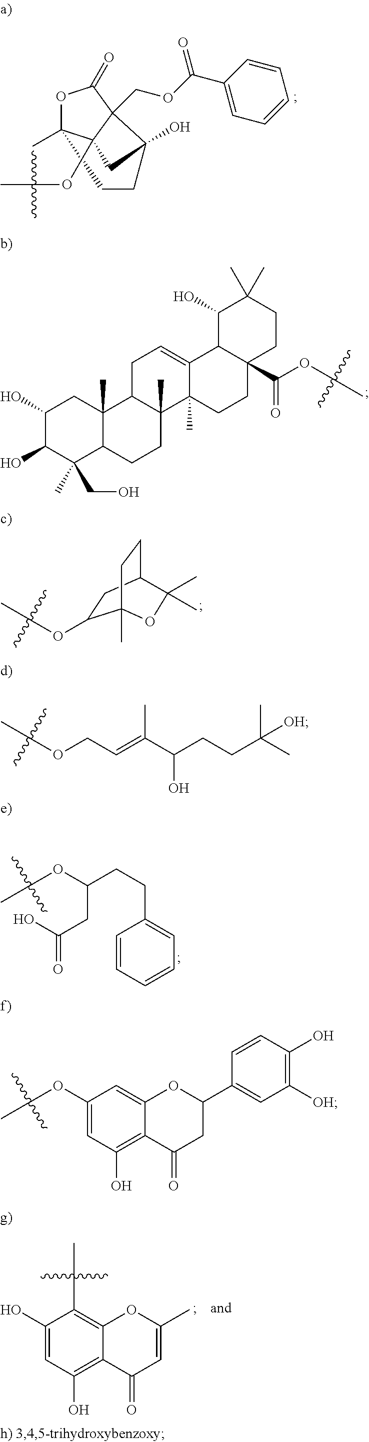

and

E is selected from the group consisting of hydrogen and substituted or non-substituted benzyl.

According to any one of the embodiments described herein, and any combination thereof, in some embodiments, the compound comprises a glucose moiety, that is, the stereochemistry of Formula III is such that the pyranose moiety depicted therein is a glucopyranose moiety). In some embodiments, the compound has the general formula:

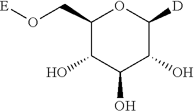

or a pharmaceutically acceptable salt thereof.

According to any one of the embodiments described herein, and any combination thereof, in some embodiments of any of the aspects described herein, the compound described herein as an SNJ2 inhibitor comprises a monosaccharide moiety (e.g., a glucose moiety), for example, a monosaccharide moiety as depicted in general formula III, attached to a terpenoid moiety. In some embodiments, the terpenoid is attached to the monosaccharide via a glycosidic bond (e.g., at the 1-position of the monosaccharide).

According to any one of the embodiments described herein, and any combination thereof, in some embodiments, the terpenoid moiety is a monoterpene derivative, that is, it comprises 10 carbon atoms derived from two isoprene units.

Exemplary terpenoid moieties include moieties a), b), c) and d) in general formula III herein. Moieties a), c) and d) are exemplary monoterpene derivatives, as described herein.

According to any one of the embodiments described herein, and any combination thereof, in some embodiments, the substituted benzyl according to general formula III is a hydroxylated benzyl, for example, a hydroxylated phenyl according to any embodiments described herein attached to a C(═O) group.

In exemplary embodiments, the benzyl is non-substituted.

According to any one of the embodiments described herein, and any combination thereof, in some embodiments, E is hydrogen.

In some embodiments of any of the aspects described herein, the compound described herein as an SYNJ2 inhibitor is a compound depicted in Table 1 in the Examples section herein, or an analog or derivative thereof, or a pharmaceutically acceptable salt thereof.

According to any one of the embodiments described herein, and any combination thereof, in some embodiments, the inhibitor is a compound depicted in Table 1 (or a pharmaceutically acceptable salt thereof) and characterized therein as having an IC50 toward sSYNJ2 of no more than 10 μM. In some embodiments, the IC50 is no more than 7 μM. In some embodiments, the IC50 is no more than 5 μM. In some embodiments, the IC50 is no more than 4 μM. In some embodiments, the IC50 is no more than 3 μM. In some embodiments, the IC50 is no more than 2 μM.

According to any one of the embodiments described herein, and any combination thereof, in some embodiments, the compound is selected from the group consisting of chlorhexidine, pyrvinium and Compound 12 as depicted below, analogs and derivatives thereof, and pharmaceutically acceptable salts thereof. Pyrvinium pamoate is an exemplary pyrvinium salt.

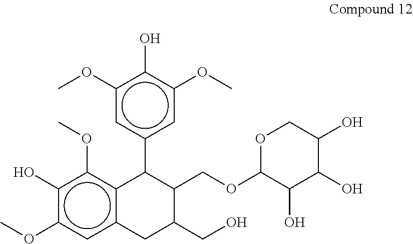

According to any one of the embodiments described herein, and any combination thereof, in some embodiments, the compound is Compound 12.

According to any one of the embodiments described herein, and any combination thereof, in some embodiments, the compound is a pharmaceutically acceptable salt of pyrvinium (e.g., pyrvinium pamoate).

For any of the embodiments described herein, the compound may be in a form of a salt, for example, a pharmaceutically acceptable salt, and/or in a form of a prodrug.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound.

In the context of some of the present embodiments, a pharmaceutically acceptable salt of the compounds described herein may optionally be an acid addition salt comprising at least one basic (e.g., amine) group of the compound which is in a positively charged form (e.g., an ammonium ion), in combination with at least one counter-ion, derived from the selected acid, that forms a pharmaceutically acceptable salt.

The acid addition salts of the compounds described herein may therefore be complexes formed between one or more amino groups of the drug and one or more equivalents of an acid.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

Depending on the stoichiometric proportions between the basic or acidic charged group(s) in the compound (e.g., amine group(s)) and the counter-ion in the salt, the acid or base additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

As used herein, the term “prodrug” refers to a compound which is converted in the body to an active compound (e.g., a SYNJ2 inhibitor described herein). A prodrug is typically designed to facilitate administration, e.g., by enhancing absorption. A prodrug may comprise, for example, the active compound modified with ester groups, for example, wherein one or more hydroxy groups of the active compound is modified by an acyl (e.g., acetyl) group to form an ester group, and/or wherein one or more carboxylic acid of the active compound is modified by an alkyl (e.g., ethyl) group to form an ester group.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The present embodiments further encompass any stereoisomers (enantiomers and diastereomers) of the compounds described herein, except in embodiments wherein a specific stereoisomer is explicitly required, as well as any isomorph thereof.

As used herein throughout, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. When substituted, the substituent group can be, for example, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein.

An “alkenyl” group refers to an alkyl group which consists of at least two carbon atoms and at least one carbon-carbon double bond.

An “alkynyl” group refers to an alkyl group which consists of at least two carbon atoms and at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, lone pair electrons, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino, as these terms are defined herein. Representative examples are piperidine, piperazine, tetrahydrofuran, tetrahydropyran, morpholine and the like.

A “hydroxy” group refers to an —OH group.

As used herein, the terms “amine” and “amino” refer to either a —NR′R″ group, wherein R′ and R″ are selected from the group consisting of hydrogen, alkyl, cycloalkyl, heteroalicyclic (bonded through a ring carbon), aryl and heteroaryl (bonded through a ring carbon). R′ and R″ are bound via a carbon atom thereof. Optionally, R′ and R″ are selected from the group consisting of hydrogen and alkyl comprising 1 to 4 carbon atoms. Optionally, R′ and R″ are hydrogen.

An “azide” group refers to a —N═N⁺=N⁻ group.

An “alkoxy” group refers to both an —O-alkyl and an —O-cycloalkyl group, as defined herein.

An “aryloxy” group refers to both an —O-aryl and an —O-heteroaryl group, as defined herein.

A “thiohydroxy” or “thiol” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “disulfide” group refers to both a —S-thioalkoxy and a —S-thioaryloxy group.

A disulfide bond describes a —S—S— bond.

A “carbonyl” group refers to a —C(═O)—R′ group, where R′ is defined as hereinabove.

A “thiocarbonyl” group refers to a —C(═S)—R′ group, where R′ is as defined herein.

A “C-carboxy” group refers to a —C(═O)—O—R′ groups, where R′ is as defined herein.

An “O-carboxy” group refers to an R′C(═O)—O— group, where R′ is as defined herein.

An “oxo” group refers to a ═O group.

A “carboxylate” or “carboxyl” encompasses both C-carboxy and O-carboxy groups, as defined herein.

A “carboxylic acid” group refers to a C-carboxy group in which R′ is hydrogen.

A “thiocarboxy” or “thiocarboxylate” group refers to both —C(═S)—O—R′ and —O—C(═S)R′ groups.

An “ester” refers to a C-carboxy group wherein R′ is not hydrogen.

An ester bond refers to a —O—C(═O)— bond.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R′S(═O)₂—NR″ group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″— group, where each of R′ and R″ is as defined herein.

A “carbamyl” or “carbamate” group encompasses O-carbamyl and N-carbamyl groups.

A carbamate bond describes a —O—C(═O)—NR′— bond, where R′ is as described herein.

An “O-thiocarbamyl” group refers to an —OC(═S)—NR′R″ group, where each of R′ and R″ is as defined herein.

An “N-thiocarbamyl” group refers to an R′OC(═S)NR″— group, where each of R′ and R″ is as defined herein.

A “thiocarbamyl” or “thiocarbamate” group encompasses O-thiocarbamyl and N-thiocarbamyl groups.

A thiocarbamate bond describes a —O—C(═S)—NR′— bond, where R′ is as described herein.

A “C-amido” group refers to a —C(═O)—NR′R″ group, where each of R′ and R″ is as defined herein.

An “N-amido” group refers to an R′C(═O)—NR″— group, where each of R′ and R″ is as defined herein.

An “amide” group encompasses both C-amido and N-amido groups.

An amide bond describes a —NR′—C(═O)— bond, where R′ is as defined herein.

A “urea” group refers to an —N(R′)—C(═O)—NR″R′″ group, where each of R′ and R″ is as defined herein, and R′″ is defined as R′ and R″ are defined herein.

A “nitro” group refers to an —NO₂ group.

A “cyano” group refers to a —C≡N group.

The term “hydrazine” describes a —N(R′)—N(R″)R′″ group, with each of R′, R″ and R′″ as defined hereinabove.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

A “phosphoric acid” is a phosphate group is which each of R is hydrogen.

The term “phosphinyl” describes a —PR′R″ group, with each of R′ and R″ as defined hereinabove.

The term “thiourea” describes a —N(R′)—C(═S)—NR″— group, with each of R′ and R″ as defined hereinabove.

According to another aspect of embodiments of the invention, there is provided a method of inhibiting synaptojanin-2, the method comprising contacting the synaptojanin-2 with an effective amount of a compound described herein as a synaptojanin-2 inhibitor.

In some embodiments, the method is effected ex vivo, for example, for research.

In some embodiments, the method is effected in vivo. In some embodiments, the method is utilized for treating a disease or disorder in which inhibition of synaptojanin-2 is beneficial (e.g., a condition described herein).

In some embodiments, an effective amount is less than 100 μM. In some embodiments, an effective amount is less than 10 μM. In some embodiments, an effective amount is less than 5 μM. In some embodiments, an effective amount is less than 2.5 μM.

In some embodiments, an effective amount is at least 100% of the IC50 of the compound towards SYNJ2. In some embodiments, an effective amount is at least 200% of the IC50 of the compound towards SYNJ2. In some embodiments, an effective amount is at least 300% of the IC50 of the compound towards SYNJ2. In some embodiments, an effective amount is at least 500% of the IC50 of the compound towards SYNJ2. In some embodiments, an effective amount is at least 1000% of the IC50 of the compound towards SYNJ2.

Non-limiting examples of cancers which can be treated according to some embodiments of any of the aspects of the invention include any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic ; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibro sarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute—megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

In some embodiments, the cancer or tumor is a glioma.

In some embodiments, the cancer or tumor is not a glioma. According to a specific embodiment, the cancer (or the cancer metastasis) is breast cancer.

According to a specific embodiment, the cancer (or the cancer metastasis) is EGF-regulated.

According to another preferred embodiment, the cancer is characterized by over-expression or up-regulation of an ErbB receptor molecule such as EGFR or HER2.

Mutations that lead to EGFR overexpression (known as upregulation) or overactivity have been associated with a number of cancers, including lung cancer, anal cancers and glioblastoma multiforme. In this latter case a more or less specific mutation of EGFR, called EGFRvIII is often observed. Mutations, amplifications or misregulations of EGFR or family members are implicated in about 30% of all epithelial cancers.

Mutations involving EGFR could lead to its constant activation, which could result in uncontrolled cell division—a predisposition for cancer. Consequently, mutations of EGFR have been identified in several types of cancer, and it is the target of an expanding class of anticancer therapies [Zang et al., J Clin Invest 117, 2051-2058 (2007)].

Amplification or over-expression of the ERBB2 gene occurs in approximately 30% of breast cancers. It is strongly associated with increased disease recurrence and a worse prognosis. Over-expression is also known to occur in ovarian, stomach, and aggressive forms of uterine cancer, such as uterine serous endometrial carcinoma.

Following is a list of cancers in which members of the ErbB family of receptor tyrosine kinases are implicated.

ErbB-1—adrenocortical cancer, biliary cancer, cervical cancer, colorectal cancer, esophageal cancer, gallbladder cancer, gastric cancer, glioblastoma, head and neck cancer, lung cancer (non-small cell, squamous cell carcinoma, adenocarcinoma, and large cell lung cancer), pancreatic cancer, salivary gland cancer, diarrhea benign neoplasm, invasive carcinoma, skin disease, ductal carcinoma in situ, paronychia.

ErbB-2—biliary cancer, bladder cancer, breast cancer, cholangiocarcinoma, esophageal cancer, gallbladder cancer, gastric cancer, glioblastoma, ovarian cancer, pancreatic cancer, salivary gland cancer. According to a specific embodiment the cancer is breast or gastric cancer.

ErbB-3—breast cancer, lung cancer and viral leukemia.

ErbB-4—breast cancer, viral leukemia, medulloblastoma, lung cancer and mammary tumor.

As described herein, according to some embodiments of various aspects described herein, the inhibitor of SYNJ2 is utilized in addition to an inhibitor of a cell surface receptor associated with an onset or progression of cancer. According to an embodiment of the invention, the receptor is an oncogene.

Examples of receptors which may be targeted according to the present teachings are receptor tyrosine kinases such as those EGFR, PDGFR, VEGFR, FGFR and ErbB-2.

Other surface molecules which can be targeted include integrins matrix metalloproteinases (MMPs), dynamin, TKS5 and CD44.

Inhibitors of cell surface molecules are well known in the art. A non-limiting list of such inhibitors is provided infra.

Thus for example, the identification of EGFR as an oncogene has led to the development of anticancer therapeutics directed against EGFR.

Cetuximab and panitumumab are examples of monoclonal antibody inhibitors. Other monoclonals in clinical development are zalutumumab, nimotuzumab, and matuzumab. The monoclonal antibodies block the extracellular ligand binding domain. With the binding site blocked, signal molecules can no longer attach there and activate the tyrosine kinase.

Another method is using small molecules to inhibit the EGFR tyrosine kinase, which is on the cytoplasmic side of the receptor. Without kinase activity, EGFR is unable to activate itself, which is a prerequisite for binding of downstream adaptor proteins. Ostensibly by halting the signaling cascade in cells that rely on this pathway for growth, tumor proliferation and migration is diminished. Gefitinib, erlotinib, and lapatinib (mixed EGFR and ERBB2 inhibitor) are examples of small molecule kinase inhibitors. Other examples include, Iressa and Tarceva directly target the EGFR.

HER2 is the target of the monoclonal antibody trastuzumab (marketed as Herceptin). Trastuzumab is effective only in cancers where HER2 is over-expressed. Another monoclonal antibody, pertuzumab, which inhibits dimerization of HER2 and HER3 receptors, was approved by the FDA for use in combination with trastuzumab in June 2012.

Additionally, NeuVax™ (Galena Biopharma) is a peptide-based immunotherapy that directs “killer” T cells to target and destroy cancer cells that express HER2.

The expression of HER2 is regulated by signaling through estrogen receptors. Estradiol and tamoxifen acting through the estrogen receptor down-regulate the expression of HER2.

Examples of antibodies which can be used according to the embodiments of the invention include, without limitation, alemtuzumab (a humanized antibody which targets CD62, and is approved for treatment of chronic lymphocytic leukemia); bevacizumab (a humanized antibody which targets vascular endothelial growth factor, and is approved for treatment of colorectal cancer); brentuximab vedotim (a chimeric antibody which targets CD30, and is approved for treatment of Hoodkin lymphoma and anaplastic large-cell lymphoma); cetuximab (a chimeric antibody which targets epidermal growth factor, and is approved for treatment of colorectal cancer); gemtuzumab ozogamicin (a humanized antibody which targets CD33, and is approved for treatment of acute myelogenous leukemia (with calicheamicin)); ibritumomab tiuxetan (a murine antibody which targets CD20, and is approved for treatment of non-Hogkin lymphoma (with yttrium-90 or indium-111)); panitumumab (a human antibody which targets epidermal growth factor, and is approved for treatment of colorectal cancer; rituximab (a chimeric antibody which targets CD20, and is approved for treatment of non-Hodgkin lymphoma; and trastuzumab (a humanized antibody which targets ErbB2, and is approved for treatment of breast cancer).

The inhibitors of the SYNJ2 described herein and optionally the inhibitor of the cell surface receptor according to any of the aspects of embodiments of the invention described herein can be administered to the subject per se or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the inhibitor of SYNJ2 (and optionally the inhibitor of the cell surface receptor) accountable for the biological effect, as described herein.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington' s Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g., SYNJ2 inhibitor) effective to prevent, alleviate or ameliorate symptoms of a disorder (e.g., cancer or metastatic cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (see, e.g., Fingl et al. (1975), in “The Pharmacological Basis of Therapeutics”, Ch. 1 p.1).

Dosage amount and interval may be adjusted individually to provide SYNJ2 inhibitor levels of the active ingredient are sufficient to induce or suppress the biological effect (minimal effective concentration, MEC). The MEC will vary for each preparation, but can be estimated from in vitro data, e.g., based on results on a SYNJ2 inhibition assay described herein. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed herein.

According to another aspect described herein, there is provided a kit for the treatment of cancer or prevention of cancer metastasis, the kit comprising a packaging material packaging a compound described herein as a SYNJ2 inhibitor and an inhibitor of a cell surface receptor associated with an onset or progression of cancer, as described herein.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A Laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Cell Migration, Invasion and Chemotaxis Assays:

Cells were plated in triplicates in the upper compartment of a Transwell tray (BD Bioscience), and allowed to migrate through the intervening membrane for 18 hours. Thereafter, cells were fixed in paraformaldehyde (3%), permeabilized in Triton X-100 (0.05%) and stained with methyl violet (0.02%). Non-migrating cells, growing on the upper side of the filter, were removed and migrated cells photographed. Invasion assays were performed using BioCoat Matrigel Chambers. For chemotaxis chambers from ibidi GmbH (Germany) and time-lapse imaging were used. The positions of cell nuclei were tracked using ImageJ.

Phosphoinositide Analyses:

Cells were incubated for 30 minutes in inositol-free medium, which was changed to medium supplemented with both [³H]-inositol and dialyzed serum (10%). Cells were cultured for three days, rinsed and extracted in 1M HCl followed by 1M Methanol. The cells were then scraped and extracted in chloroform, and then in methanol:0.1M EDTA pH8.0, and the organic phase evaporated. Thereafter, extracts were de-acetylated, separated by anionic-exchange HPLC (Agilent 1200) using two partisphere SAX columns (Whatman) in tandem, and a four-step gradient of ammonium phosphate pH 6.0. The radiolabelled eluate was detected by an online flow scintillation analyzer and quantified using ProFSA software (Perkin-Elmer).

Gelatin Zymography:

To detect MMP-2 activity, biological samples were separated electrophoretically on 10% polyacrylamide/0.1% gelatin-embedded gels. The gels were then washed in 2.5% Triton X-100, and incubated at 37° C. for 36 hours in 50 mM Tris-HCl (pH 7.5), containing 0.2 M NaCl, 5 mM CaCl₂, 1 μM ZnCl₂, 0.02% Brij 35, and 1 mM p-aminophenylmercuric acetate.

Metastasis Tests in Animals:

Female CB-17 SCID mice (Harlan Laboratories, Haslett, Mich.; 15 per group) were implanted in the fat pad with MDA-MB-231 cells (1.4 ×10⁶ cells/mouse). Two and six weeks post implantation, mice were anesthetized, tumor sizes were measured and metastases in lymph nodes were visualized using a fluorescent binocular. For lung metastases, mice were sacrificed, lungs were removed, washed, and images were acquired using a fluorescent binocular. Two-sided Fischer' s exact test was used for analysis of lymph node metastasis. Tumor growth measurements used the Exact-sig [2×1-tailed]) Mann-Whitney test.

Reagents:

Unless indicated, Human recombinant growth factors and other materials were purchased from Sigma (St. Louis, Mo., USA). Radioactive materials and a chemiluminescence kit for immunoblotting were obtained from Amersham (Buckinghamshire, UK). The EGFR-kinase inhibitor AG1478, MEK inhibitor U0126 and the PI3K inhibitor Wortmannin were from Calbiochem (San Diego, Calif.). Plates for wound-healing assays were from ibidi GmbH (Munich, Germany). 35-mm glass-bottom dishes for time-lapse imaging were purchased from MaTek (Ashland, Mass.). Murine monoclonal antibody (mAb) 111.6 to the EGF-receptor was generated in our laboratory. Anti-EGFR for western blot analysis was from Alexis (Lausen, Switzerland). Anti Ras-GAP and anti-AKT antibodies were from Santa Cruz Biotechnology (Santa Cruz, Calif.). Anti-EEA1, anti-Rab5, anti-Rab4, and anti Rac1 were from BD Transduction Laboratories (Franklin Lakes, N.J.). Anti-SYNJ2 mAb was from Abnova (Taipei, Taiwan). The following secondary antibodies were used: goat anti-mouse IgG and goat anti-rabbit IgG antibodies conjugated to Horseradish peroxidase (HRP) were purchased from Jackson ImmunoResearch Laboratories (Bar Harbor, Me.). Texas-red transferrin, goat anti mouse Alexa-488, Alexa-555 and Alexa-647 secondary antibodies were from Invitrogen (Carlsbad, Calif.).

siRNA control was from “Thermo scientific Dharmacon” cat. D-001810-10-05; siRNA sequence against SYNJ2 is GGACAGCACUGCAGGUGUU; all shRNA were from SIGMA Israel: shRNA control-cat. SHC002; shRNA sequences against SYNJ2 used is CCGGCCGGAAGAACAGTTTGAGCAACTCGAGTTGCTCAAACTGTTCTTCCG GTTTTTG.

Cell Lines and Transfections:

MCF10A cells were grown in DMEM:F12 (1:1) medium supplemented with antibiotics, insulin (10 μg/mL), cholera toxin (0.1 μg/mL), hydrocortisone (0.5 μg/mL), heat-inactivated horse serum (5% vol/vol), and EGF (10 ng/mL). Human mammary MDA-MB-231 cells were grown in RPMI-1640 (Gibco BRL; Grand Island, N.Y.) supplemented with 10% heat-inactivated fetal calf serum (Gibco), 1 mM sodium pyruvate and a penicillin-streptomycin mixture (100 unit/ml; 0.1 mg/ml; Beit Haemek, Israel). The MDA-MB-231-RFP stable cell-line was a kind gift from Prof. Hadasa Degani (The Weizmann Institute of Science, Israel). Plasmid transfections were performed using Fugen-HD according to the manufacture's guidelines (Roche, Mannheim, Germany). Alternatively, for transient mRNA knockdown experiments using siRNA oligonucleotides, cells were transfected with Oligofectamine (Invitrogen).

Lentiviral Vectors and Virus Production:

Non-targeted shRNA hairpins (control) and hairpins directed against human SYNJ2 were produced in HEK-293T cells following the manufacture's guidelines (Sigma). Target cells were infected with shRNA-encoding lentiviruses supplemented with polybrene (8 μg/mL), and cultured in the presence of puromycin (2 μg/mL) for 4 days. Stable gene-specific delivery of human SYNJ2 was performed using the ViraPower lentiviral expression system (Invitrogen), following the manufacture's guidelines.

Immunofluorescence and Image Processing:

Cells were grown on fibronectin-coated cover slips for 48 hours. Following treatments, cells were washed, permeabilized using 0.02% Triton X-100 and 3% paraformaldehyde, and fixed for 20 minutes. Confocal microscopy was performed using either a Zeiss LSM-710 microscope, or a spinning disk microscope (Zeiss 100×, NA 1.45; Yokogawa CSU-22; Zeiss fully automated, inverted 200 M; Photometrics HQ-CCD camera) and solid state lasers (473, 561 and 660 nm, exposure times: 0.25-1 sec), under the command of Slidebook™. 3D image stacks were acquired every 70-300 ms along the Z-axis by varying the position of the piezo electrically controlled stage (step size: 0.1-0.4 μm). Alternatively, live cell fluorescence microscopy was carried out using the DeltaVision system (Applied Precision, Issaqua, Wash.) and images were processed using the prism software.

Radiolabeling of EGF:

Human recombinant EGF was labeled with IODOGEN as follows: EGF (5 μg) was mixed in an Iodogen-coated tube (1 mg of reagent) with Na¹²⁵I (1 mCi). Following 15 minutes of incubation at 23° C., albumin was added to a final concentration of 0.1 mg/ml, and the mixture was separated on an Excellulose GF-5 column.

Receptor Down-Regulation Assay:

MDA-MB-231 cells were seeded in triplicates for each time point in 24-well plates, with an additional well plated for control. 48 hours later, cells were starved for 4 hours and stimulated with EGF (2 ng/ml) at 37° C. for the indicated time intervals. Subsequently, they were placed on ice, rinsed once with binding buffer (DME medium, albumin 1%, Hepes 20 mM, pH 7.5), and subjected to mild acid/salt wash (0.2 M Na Acetate buffer pH 4.5, 0.5 M NaCl) to remove surface-bound EGF. Thereafter, cells were incubated with a radiolabelled EGF for 1.5 hours at 4° C. and rinsed with binding buffer. The control well was incubated with a radiolabelled EGF and an excess of unlabelled EGF. Finally, cells were lysed with 1M NaOH, and radioactivity was determined using a γ-counter. Data represent the percentage of receptors on the cell surface relative to time 0.

Determination of Surface EGF-Receptor:

Cells (2×10⁴/well) were seeded in triplicates in 24-well plates, with an additional well plated for control. Thereafter, cells were incubated with a radiolabelled EGF for 1.5 hours at 4° C. and rinsed with binding buffer. The control well was incubated with a radiolabelled EGF and an excess of unlabelled EGF. Finally, cells were lysed in 1M NaOH solution and radioactivity was determined. Data represent the percentage of receptors on the cell surface relative to control cells.

Immunoblotting Analysis:

Cells were washed briefly with ice-cold saline, and scraped in a buffered detergent solution (25 mM HEPES (pH 7.5), 150 mM NaCl, 0.5% Na-deoxycholate, 1% NP-40, 0.1% SDS, 1 mM EDTA, 1 mM EGTA, 0.2 mM Na₃VO₄ and a protease inhibitor cocktail diluted at 1:1000). For equal gel loading, protein concentrations were determined by using the BCA (Pierce) reagent. Following gel electrophoresis, proteins were transferred to a nitrocellulose membrane. Membranes were blocked in TBST buffer (0.02 M Tris-HCl (pH 7.5), 0.15 M NaCl and 0.05% Tween 20) containing 10% low-fat milk, blotted with a primary antibody for 1 hour, washed with TBST and incubated for 30 minutes with a secondary antibody conjugated to HRP.

Wound Healing (Scratch) Assays:

Wound healing assays were performed according to manufacturer's protocol (ibidi GmbH, Germany). Briefly, MCF10A cells were trypsinized, re-suspended in EGF-deprived medium (7.0×10⁵ cells/mL) and 70 μl plated into each well, resulting in a confluent layer within 24 hours. Thereafter, Culture-Inserts were removed by using sterile tweezers and cells were allowed to migrate for 2 hours.

Scanning and Transmission Electron Microscopy:

Cells were fixed in saline supplemented with 4% paraformaldehyde and 2% sucrose. Samples were washed and subjected to a second fixative (3% paraformaldehyde and 2.5% glutaraldehyde in 0.1 M cacodylate buffer supplemented with 1% sucrose and 5 mM CaCl₂, pH 7.4). Cells were washed in 0.1 M cacodylate buffer and post-fixed with 1% osmium tetroxide in cacodylate buffer for 1 hour. For scanning electron microscopy (SEM), the post-fixed samples were washed twice and treated with 1% tannic acid for 5 minutes followed by another wash and treatment with 1% uranyl acetate for 30 minutes. Samples were dehydrated in graded ethanol, and made conductive by sputtering with a gold-palladium film. The samples were photographed using a scanning electron microscope (Leo Supra 55/Vp Zeiss, Thornwood, N.Y.).

Receptor Recycling Assay:

MDA-MB-231 cells were pre-incubated for 30 minutes at 37° C. with Alexa Fluor 488-transferrin (25 μg/ml in serum-free medium) or for 10 minutes with Alexa Fluor 488-EGF (40 ng/mL). Surface-bound ligands were detached by incubation for 30 minute at 4° C. in an acidic buffer (150 mM NaCl, 1 mM MgCl₂, 0.125 mM CaCl₂, 0.1M glycine), prior to transfer to 37° C. for the indicated time intervals, to allow for recycling of the internalized ligands. Cells were analyzed either by imaging or by FACS.

Real-Time Cell Impedance Analysis:

Measurements of cell spreading and adhesion were recorded by using the RTCA-Xcelligence System (Roche Diagnostics, Mannheim, Germany). Gold microelectrode E-plates-16 were washed once in saline. Cells (2,500 per well) were first seeded and then impedance data (cell index; derived as a relative change in measured electrical impedance) was recorded in the indicated intervals. The data was analyzed using software package 1.2 provided by the manufacturer.

TAPP1-PH Domain Expression and Purification:

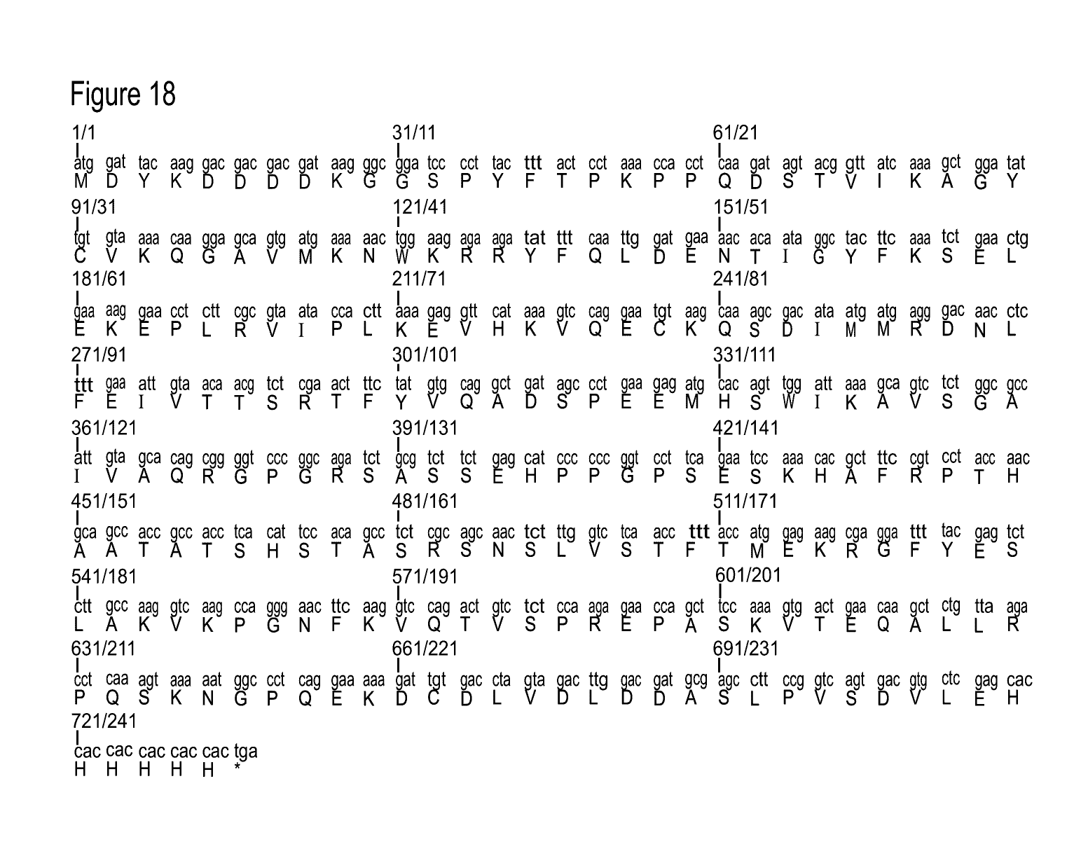

A construct encoding two TAPP1-PH domains was cloned in tandem (FIG. 18) into pET28 plasmid containing an N-terminal Flag tag and C-terminal 6× His tag and expressed in E. coli BL21(DE3) following induction with 200 μM IPTG. The bacteria was grown at 15° C. and then lysed with a cell disrupter. Cell debris was removed by centrifugation and the protein was captured on a Ni column (HisPrep FF 16/10, GE Healthcare) equilibrated with 50 mM Tris pH 8, 0.5 M NaCl and 20 mM imidazole. The protein was eluted in the same buffer containing 0.5 M Imidazole. Fractions containing the TAPP1-PH domain were injected into a size exclusion column (Hiload_26/60_Superdex 75, GE Healthcare) equilibrated with buffer containing 50 mM Tris pH 8 and 100 mM NaCl. The pooled peak containing TAPP1-PH domain was diluted three fold with 20 mM sodium phosphate buffer pH 7.2 and loaded onto a cation exchange column (HiTrap_SP_FF_5ml, GE Healthcare) equilibrated with the same phosphate buffer. The pure protein was eluted from the column with a linear gradient of the phosphate buffer containing 1 M NaCl (TAPP1-PH domain elutes at 200 mM NaCl). The fractions containing the pure TAPP1-PH domain as evaluated by SDS-PAGE were pooled together and protein concentration was determined by Bradford reagent and OD₂₈₀ (extinction coefficient of 20,520) quantization. The protein was divided into aliquots, flash frozen with liquid nitrogen and stored at −80° C.

5′ Phosphatase Activity of SYNJ2 and SYNJ1:

Measurements of the ability of SYNJ2 to hydrolyze the 5-phosphate from PI(3,4,5)P3 to generate PI(3,4)P2 were recorded by a competitive assay, based on fluorescence polarization as a read out. Stabilizing SOP lipid mix (×50) was prepared in a glass tube by adding 100 μl of SOPS (Avanti Inc., 50 mg/ml in chloroform) and 50 μl Cholesterol (Sigma Aldrich, 10 mg/ml in chloroform). The mix was air-dried using gentle nitrogen steam to evaporate the chloroform. The evaporated lipid mix was then re-suspended in 10 ml of 0.25 mg/ml C₁₂E₈ (Avanti Inc.) by 1 minute vortex at room temperature. A reaction mix comprising PBS, DTT, MgCl2 (all from Sigma Aldrich), SOP lipid mix (×50), full length purified SYNJ2 (OriGene, cat no. TP315160) and PI(3,4,5)P3 (Echelon Bioscience, cat no. P-3908), with or without a tested compound. Once PI(3,4,5)P3 was added, the reaction mix was incubated in 33° C. for 8 minutes to allow production of PI(3,4)P2 by SYNJ2 5′-phosphatase activity. Following incubation the reaction was stopped by adding a detection mix comprising PBS, DTT, detector proteins (PH domain of TAPP1), SOP lipid mix (×50), fluorescently-labeled PI(3,4)P2 (Echelon Bioscience, cat no. C34M6) and EDTA (Sigma Aldrich). Fluorescence polarization was measured using an appropriate plate reader and filter set compatible with BODIPY® TMR dye (550 nm excitation/580 nm polarizing emission filters). Unlabeled PI(3,4)P2 control was purchased from Echelon Bioscience (Cat no. P-3408).

SYNJ1 activity was assayed as described above, except that purified SYNJ1 was used instead of SYNJ2.

### Example 2

### EGF-Induced Elevated Expression of SYNJ2 Promotes Mammary Cell Invasion

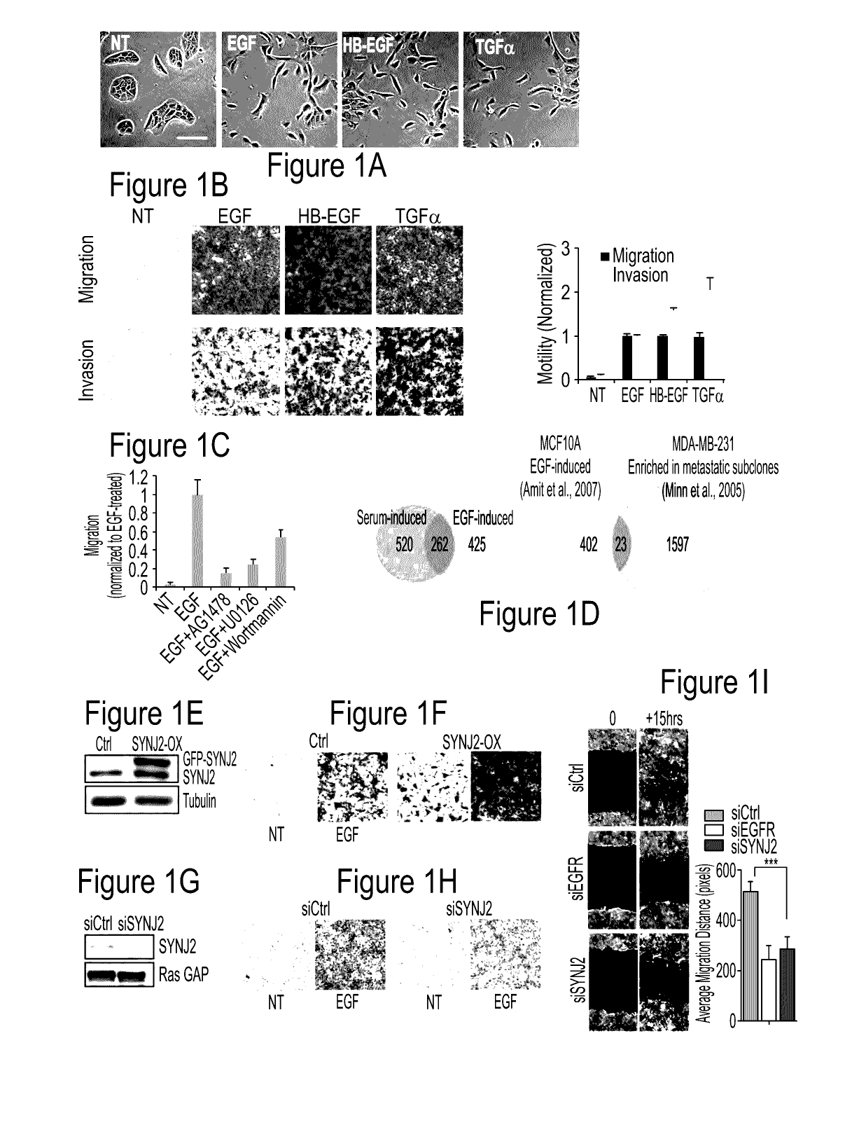

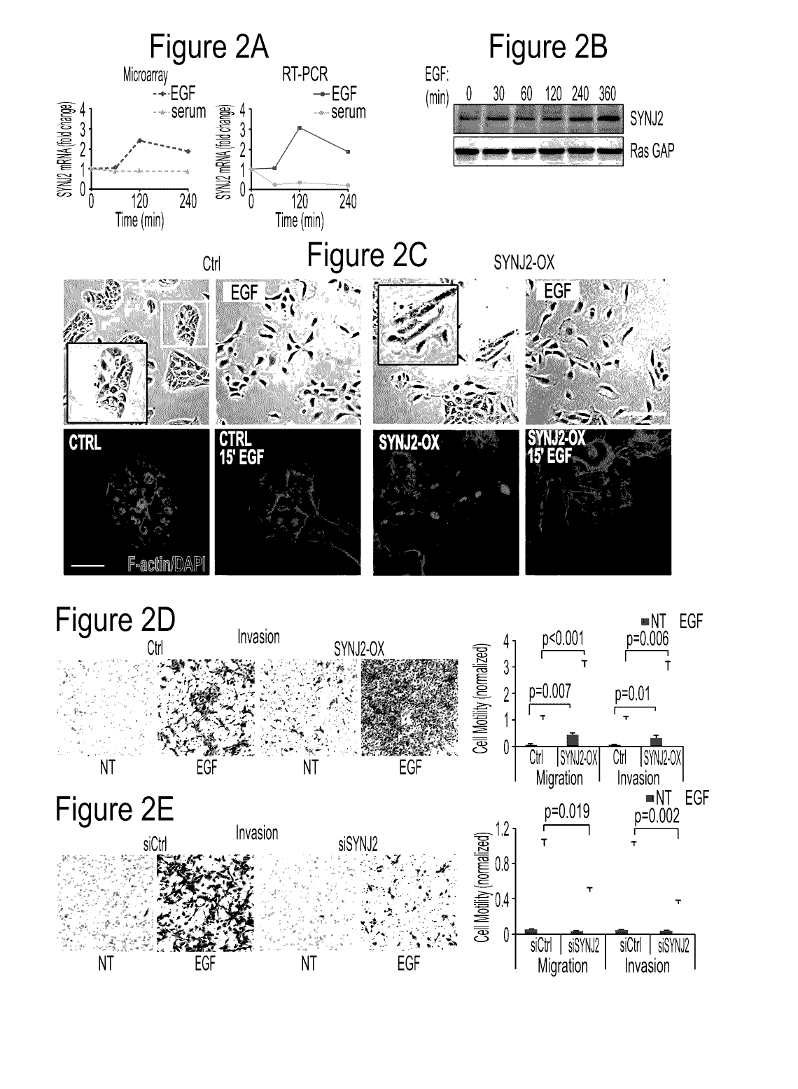

Human mammary epithelial cells (MCF10A) exhibit strong migratory and invasive phenotypes when cultured with EGF family ligands (FIGS. 1A and 1B), but treatment with serum is insufficient to propel cell motility. Co-incubation of EGF along with inhibitors of EGFR (AG1478), MEK (U0126) or PI3K (Wortmannin) reduced motility (FIG. 1C), suggesting that both MEK/ERK and PI3K activities are essential for EGF-induced migration. Importantly, the EGFR-induced motile phenotype associates with transcriptional up-regulation of 425 genes [Amit et al., Nat Genet 39, 503-512 (2007)]. To identify genes that propel metastasis, this gene-set was intersected with a larger set of genes that undergo up-regulation during in vivo selection of metastatic sub-clones of breast cancer cells [Minn et al., Nature 436, 518-524 (2005)]. The group of 23 overlapping genes (FIG. 1D) included the gene encoding Synaptojanin-2 (SYNJ2), a lipid phosphatase implicated in glioma cell invasion [Chuang et al., Cancer Research 64, 8271-8275 (2004)]. EGF-induced up-regulation of SYNJ2 was validated by PCR and immunoblotting (FIGS. 2A and 2B).

Next, MCF10A cells were transformed and sub-cloned to stably overexpress SYNJ2 (as a GFP fusion; SYNJ2-OX, FIG. 1E). When plated in EGF-deprived medium, SYNJ2-OX cells displayed a pro-migratory phenotype characterized by membrane ruffling (FIG. 2C), along with enhanced basal and EGF-induced migratory and invasive capacities (FIGS. 2D and 2C). Conversely, knockdown of SYNJ2 using small interfering RNAs (siRNAs; FIG. 1G) significantly reduced cell invasion, as well as individual and collective migration (FIGS. 2E, 1H and 1J). In conclusion, EGF-induced up-regulation of SYNJ2 drives a robust invasive phenotype of mammary cells.

### Example 3

### The Phosphatase Activity of SYNJ2 is Essential for Invasiveness of Mammary Cells

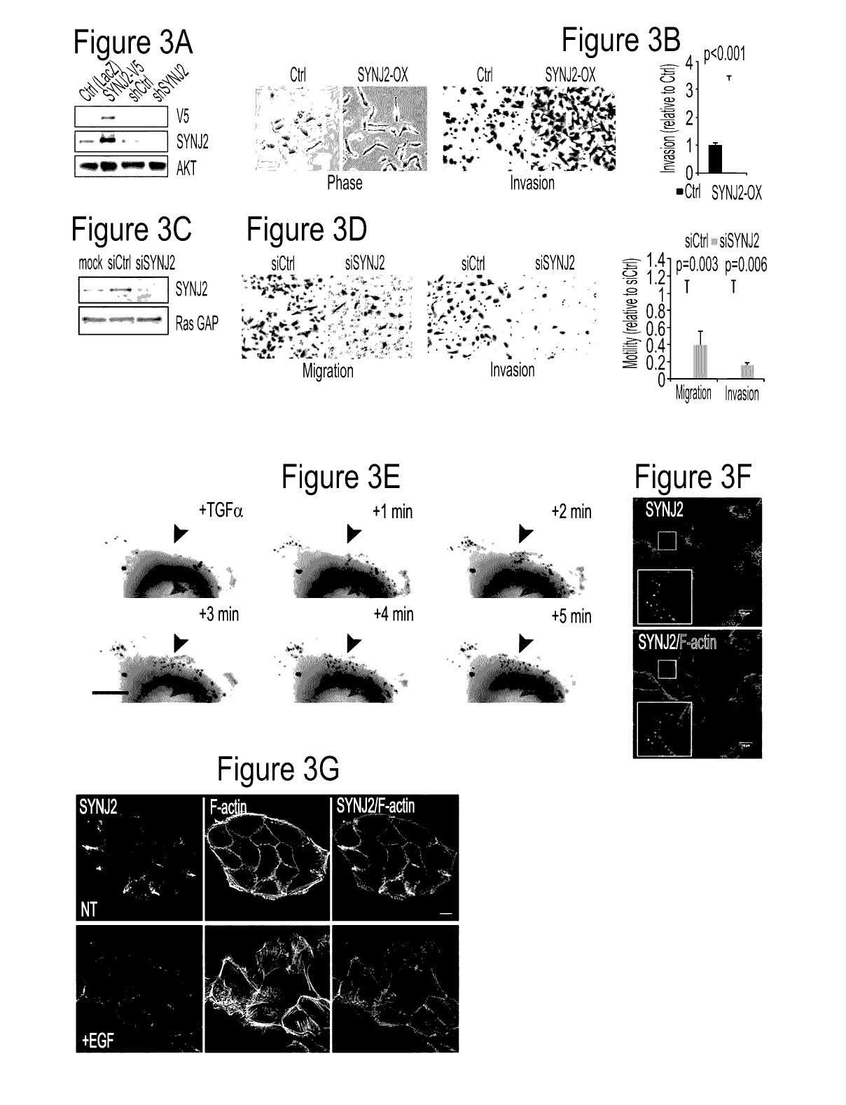

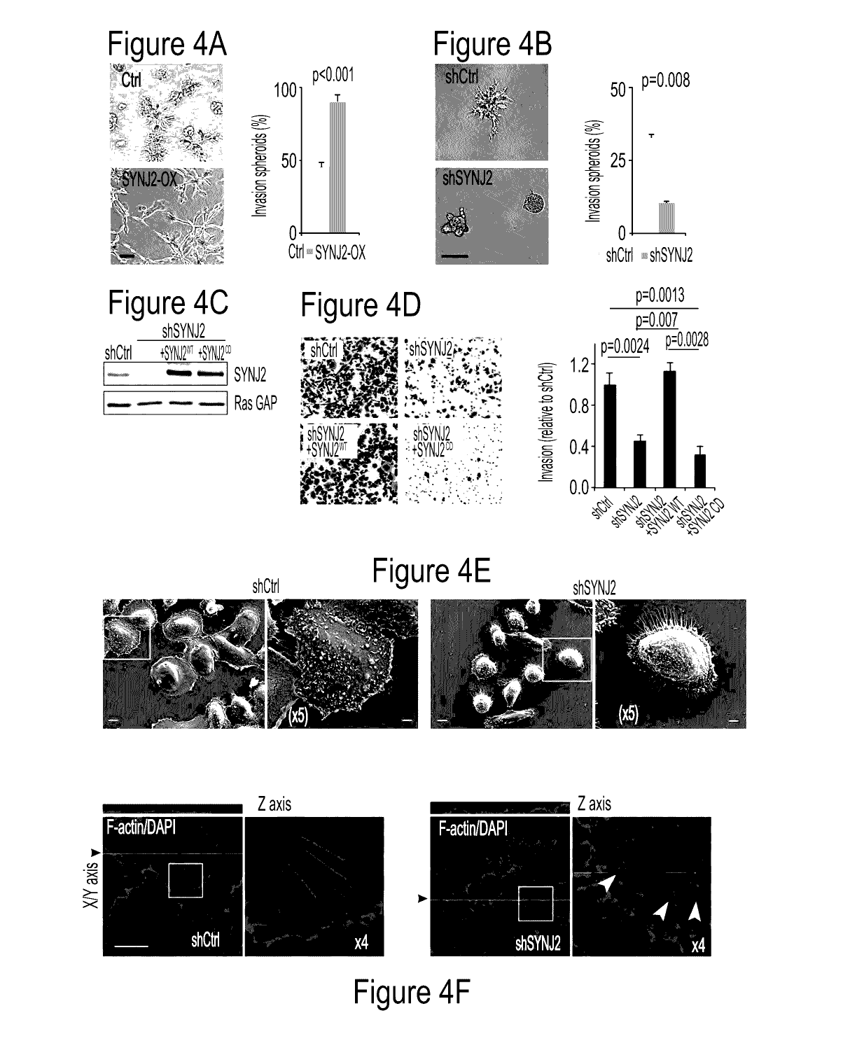

To enable in vivo experiments, the highly metastatic MDA-MB-231 breast cancer Red fluorescent protein (RFP) expressing cells were used to generate subclones overexpressing either SYNJ2 or LacZ (control), as well as sub-clones expressing shControl or SYNJ2-specific hairpins (shSYNJ2; FIG. 3A). Enhanced expression of SYNJ2 conferred an elongated morphology in 2D cultures (FIG. 3B) and extensive invasive arms, when cells were grown in 3D cultures (FIG. 4A). Conversely, SYNJ2 knockdown abrogated invasive patterns (FIG. 4B). Similarly, over-expression enhanced invasive capacities by ˜3.2 fold (FIG. 3B), and knockdown (FIG. 3C) inhibited migration and invasion (FIG. 3D). To examine roles for the catalytic phosphatase activity, shSYNJ2 cells with lentiviral particles encoding either a WT SYNJ2 or a catalytically-dead form (D388A and D726A; FIG. 4C) harboring point mutations in each of the conserved WXGDXN(F/Y)R motifs [Jefferson & Majerus, Biochemistry 35, 7890-7894 (1996)] within the phosphatase/nuclease domain (Pfam: PF03372). Unlike WT SYNJ2, re-expression of the mutant failed to restore the invasive capacity (FIG. 4D), indicating that the phosphatase activity of SYNJ2 is essential for the invasive phenotype.

The failure of shSYNJ2 cells to migrate was further supported by both scanning electron microscopy (FIG. 4E) and F-actin staining, which revealed severe actin organization defects and an increase in cell height (FIG. 4F). Importantly, also noted were actin patches clustered around circular moieties (FIG. 4F; arrowheads). Accordingly, time-lapse microscopy analyses of shSYNJ2 cells confirmed the existence of abnormal intracellular vesicles, suggesting that SYNJ2 knockdown derailed vesicular trafficking. Next, the sub-cellular localization of SYNJ2 was examined. Time-lapse images of MDA-MB-231 cells expressing GFP-SYNJ2 (FIG. 3E), as well as immunofluorescence using anti-SYNJ2 antibodies (FIG. 3F), reflected two major patterns of SYNJ2 distribution: small peripheral assemblies, which localized to the leading edge (black arrowheads in FIG. 3E), and a second population of larger assemblies, which were located closer to the cell centre (blue arrowheads). Notably, shortly after stimulation of MDA-MB-231 cells with an EGFR ligand (TGF-alpha), SYNJ2 rapidly assembled at the base of emerging lamellipodia, underneath the forming leading edge (FIGS. 3E, 3F). Interestingly, similar analyses performed with MCF10A cells indicated that SYNJ2 initially co-localizes with F-actin at cell-to-cell junctions, but translocates to the leading edge, typically to the base of lamellipodia, upon stimulation with EGF (FIG. 3G). In conclusion, these observations indicate that growth factors regulate not only the levels of SYNJ2 expression, but also its dynamic recruitment to the leading edge.

### Example 4

### Recruitment of SYNJ2 to the Ventral Membrane Depends on Dynamin and Rac1

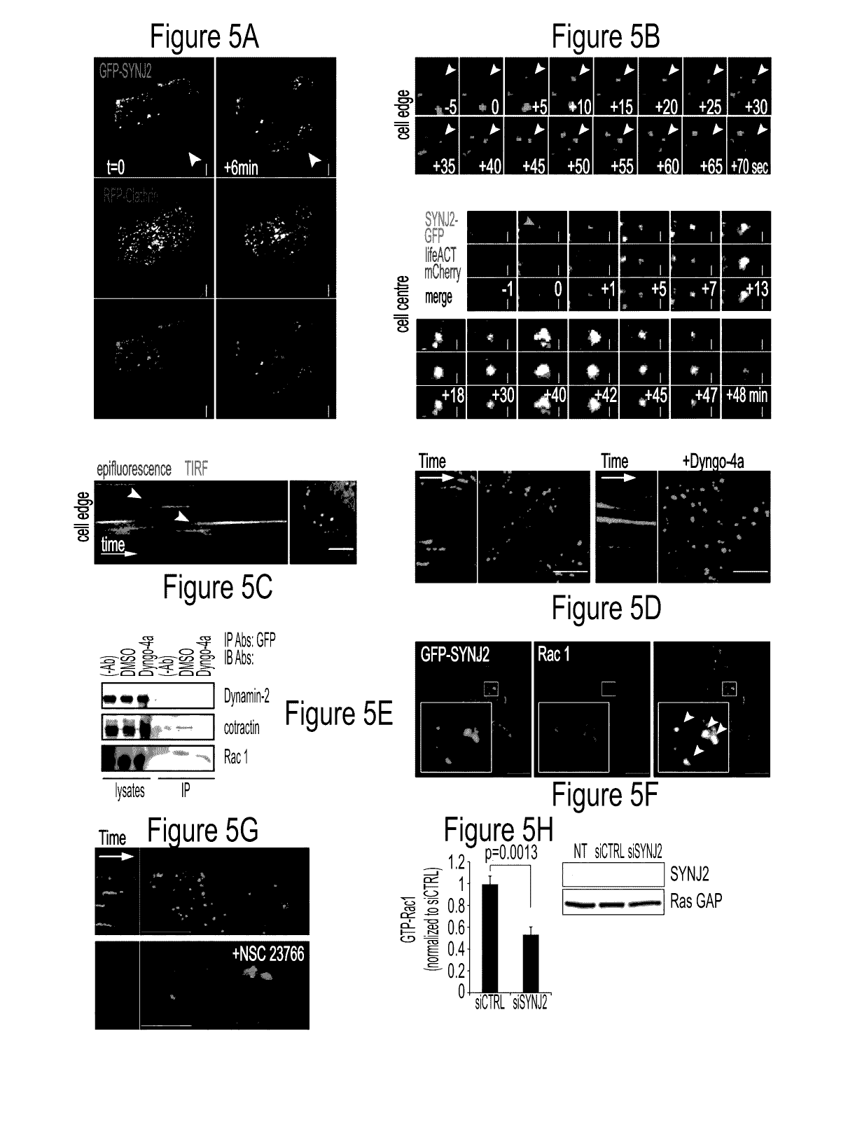

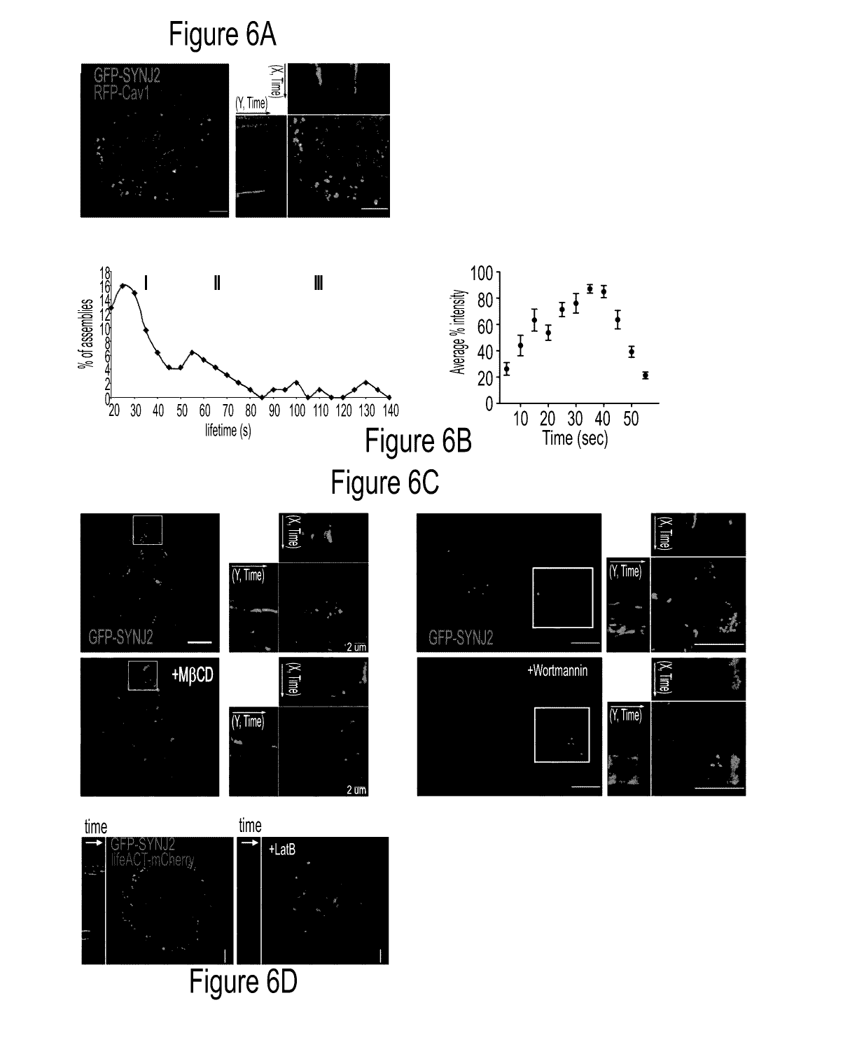

To investigate the dynamics of SYNJ2's sites of localization, a stably expressing GFP-SYNJ2 MDA-MB-231 subclone (GFP-SYNJ2 cells) was generated and analyzed for the formation and consumption of GFP-SYNJ2 puncta. These were classified into kinetically distinct sub-populations: dynamic puncta that localized to ruffling membranes and puncta localized to discrete regions proximal to the cell centre (FIG. 5A). Notably, GFP-SYNJ2 puncta showed minimal overlap with assemblies marked by RFP-Clathrin light chain A (FIG. 5A) or RFP-Caveolin 1 (FIG. 6A), suggesting minor localization to Clathrin-coated pits or to caveolae. Importantly, newly formed peripheral puncta heralded nascent lamellipodia, as their appearance preceded local formation of lamellipodia. In contrast, the more central and stable clusters of puncta, which co-localized with actin, persisted for ˜30 minutes (FIG. 5B). Accordingly, tracking of individual assemblies (FIG. 6B; left) revealed remarkably wide distribution of lifetimes: short-lived (˜20-40 s, 60% of assemblies), intermediate lifetimes, and long-lived assemblies (˜10% of assemblies). Initiation of the intermediate group was followed by a continuous increase in fluorescence intensity, while the assembly remained static in terms of movement (FIG. 6B; right). This dynamic pattern resembles that of Clathrin-coated pits [Ehrlich et al., Cell 118, 591-605 (2004)] and suggests the formation and consumption of trafficking intermediates.

The mostly bimodal compartmentalization of GFP-SYNJ2 at the ventral membrane was reinforced by the synchronous appearance and disappearance of fluorescence signals in experiments employing both epifluorescence (red; relatively insensitive to changes in the Z dimension) and total internal reflection microscopy (TIRF, green; limited to ˜200 nm depth). Because puncta appeared yellow throughout their lifetime (FIG. 5C), the present inventors concluded that SYNJ2 assembles within the plane of the ventral plasma membrane. By employing a panel of inhibitors it was found that the assembly was dramatically inhibited by cholesterol depletion (FIG. 6C; left), suggesting that cholesterol-rich membrane microdomains are needed for SYNJ2 recruitment to the ventral membrane. A similar inhibitory effect was induced by Wortmannin (FIG. 6C; right), suggesting a role for PI3K. Another requirement was revealed by employing Dyngo-4a, an inhibitor of Dynamin, the large GTPase that mediates the scission step of clathrin-dependent and clathrin-independent carriers, and whose inhibition leads to accumulation of U-shape invagination intermediates [Macia et al., Dev Cell 10, 839-850 (2006)]. Because Dyngo-4a strongly arrested the dynamic assemblies of SYNJ2 at the plasma membrane (FIG. 5D), the present inventors concluded that SYNJ2 is recruited to nascent trafficking intermediates regulated by Dynamin. Because Dynamin has been implicated as a facilitator of cell migration and invasion [Kruchten & McNiven, J Cell Science 119, 1683-1690 (2006)], its physical interactions with SYNJ2 was tested. This experiment confirmed complex formation between active Dynamin and SYNJ2 (FIG. 5E), in line with an extended role for Dynamin in both endocytosis and actin-based migration.

SYNJ2 can physically interact with GTP-loaded Rac1 [Malecz et al., Curr Biol 10, 1383-1386 (2000)], and inducible activation of Rac1 requires internalization and subsequent recycling [Palamidessi et al., Cell 134, 135-147 (2008)]. Hence, the coincidence of the peripheral puncta of SYNJ2 coincide with Rac1 was tested. Indeed, immunostaining of endogenous Rac1 revealed co-localization with peripheral puncta of GFP-SYNJ2 (FIG. 5F). Moreover, inhibition of GTP loading onto Rac1 (using NSC-23766) dramatically reduced the number of GFP-SYNJ2 puncta (FIG. 5G). Complementarily, SYNJ2 knockdown reduced the levels of GTP-loaded Rac1 in MDA-MB-231 cells (FIG. 5H). In accord with a regulatory role for Rac1 and the actin cytoskeleton in recruiting SYNJ2 to the membrane, inhibition of actin dynamics with Latrunculin abrogated GFP-SYNJ2 dynamics (FIG. 6D). Taken together, these results associate the peripheral SYNJ2 assemblies, with a dynamin-mediated endocytic pathway that depends on cholesterol, 3′-phosphoinositides, actin and active Rac1. Notably, this pathway shares several attributes with clathrin-independent carriers that enable rapid membrane and adhesion turnover at the leading edge of migrating fibroblasts [Howes et al., J Cell Biology 190, 675-691 (2010)].

### Example 5

### SYNJ2 Controls Vesicular Trafficking of Cell Surface Receptors

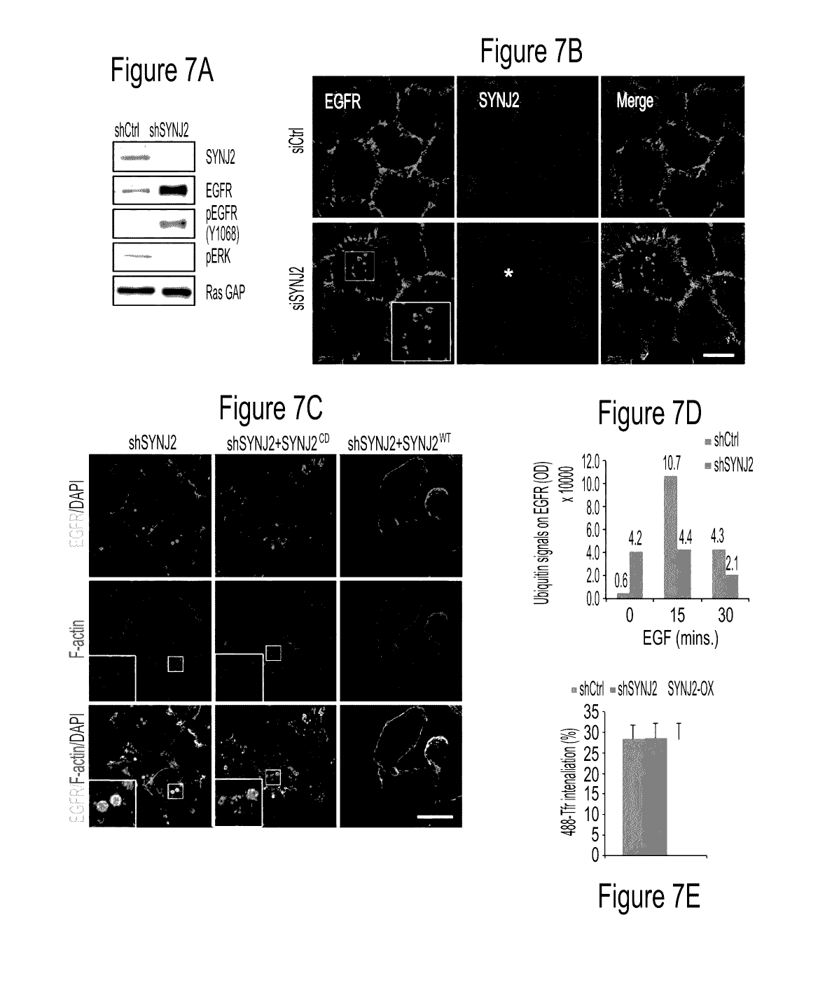

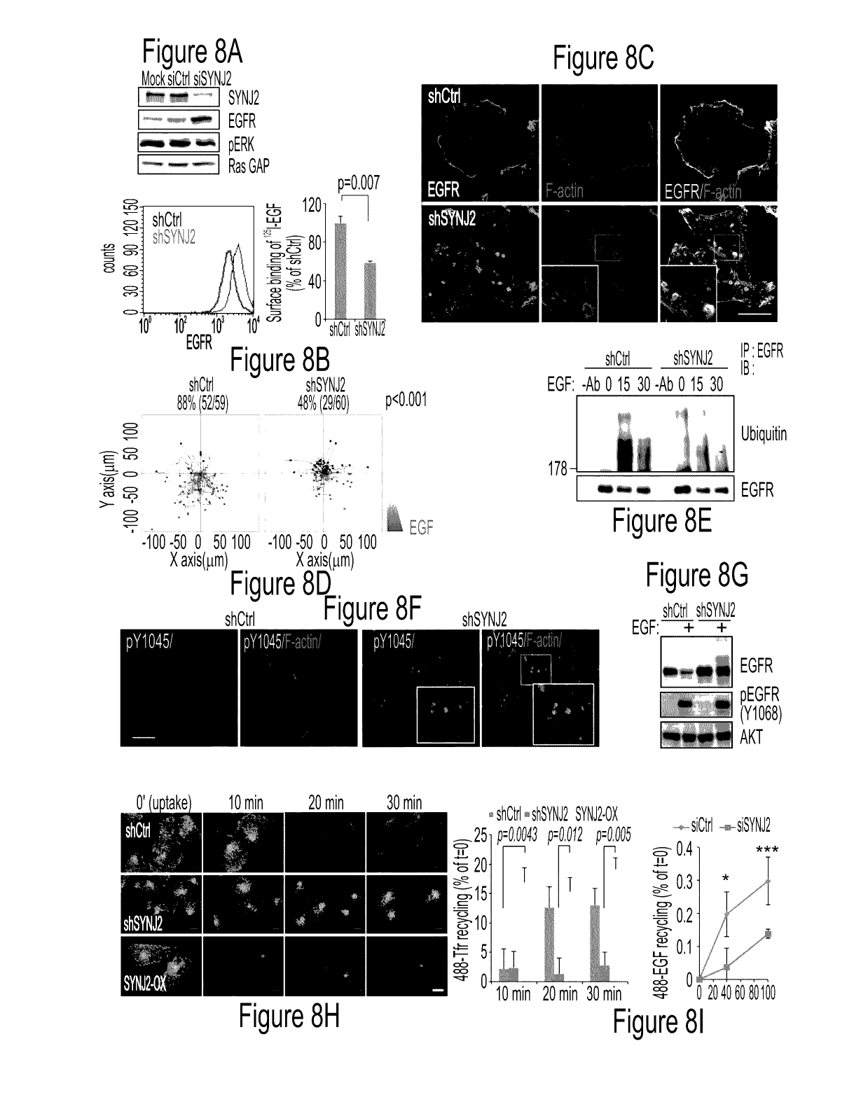

Although EGF-treated shSYNJ2-MCF10A cells displayed higher levels of total and phosphorylated EGFR relative to control cells, this translated to lower, rather than higher activation of ERK (FIG. 7A). Along this line, it was noted that SYNJ2 knockdown trapped EGFRs in enlarged intracellular vesicles (FIG. 7B). Consistent with trapping, immunoblotting of MDA-MB-231 cells similarly revealed that EGFR levels were stabilized in siSYNJ2 cells (FIG. 8A), but quantification of surface EGFR by using two methods indicated significantly lower surface levels (FIG. 8B). Intracellular trapping of EGFR bears functional consequences: in line with their well-characterized chemotactic function [Mouneimne et al., Curr Biol 16, 2193-2205 (2006); van Rheenen et al., J Cell Biology 179, 1247-1259 (2007)], EGFRs localized to the leading edge of mammary cells, but EGFRs of shSYNJ2 cells lost their polarized distribution and accumulated in large, actin-decorated vesicles (FIG. 8C). Notably, EGFR trafficking defects observed in shSYNJ2 cells could be rescued by WT SYNJ2, but not by a catalytically-dead form (FIG. 7C), indicating that the phosphatase activity of SYNJ2 is essential for vesicular trafficking of EGFRs to and from the leading edge, where it mediates the chemotactic response to gradients of EGF. Consistent with this model, shSYNJ2 cells severely lost the ability to migrate along a gradient of EGF (FIG. 8D).

The abnormal accumulation of EGFR in SYNJ2-depleted cells could reflect defects in EGFR delivery, arrested recycling, or impaired sorting for degradation, a process regulated by ubiquitination [Goh et al., J Cell Biology 189, 871-883 (2010)]. Consistent with impaired sorting, SYNJ2-depleted cells exhibited significantly higher basal EGFR ubiquitination, which was only weakly altered in response to EGF (FIGS. 8E and 7D). Furthermore, despite being tagged for degradation by phosphorylation of tyrosine 1045 (a docking site for the ubiquitin ligase c-Cbl; FIG. 8F), an EGF stimulation experiment confirmed normal activation (tyrosine 1068 phosphorylation) but defective degradation in shSYNJ2 cells (FIG. 8G). To address a recycling defect, fluorescent ligands were employed to follow the extensive recycling of the transferrin receptor (TfR), as well as the weaker recycling of EGFR. Although TfR internalization was not affected, recycling was markedly decreased in shSYNJ2 cells and, conversely, markedly accelerated in SYNJ2-OX cells (FIGS. 8H and 7E). Likewise, flow cytometry analyses indicated defective recycling of fluorescent-EGF (FIG. 8I), and live cell imaging confirmed ligand accumulation within the large vesicles of SYNJ2-depleted cells. In conclusion, these results indicate that SYNJ2 is essential for proper recycling of both EGFR and TfR.

### Example 6

### SYNJ2 Knockdown Perturbs Homeostasis of Phosphoinositol Lipids and Alters Both Endocytosis and Adhesion

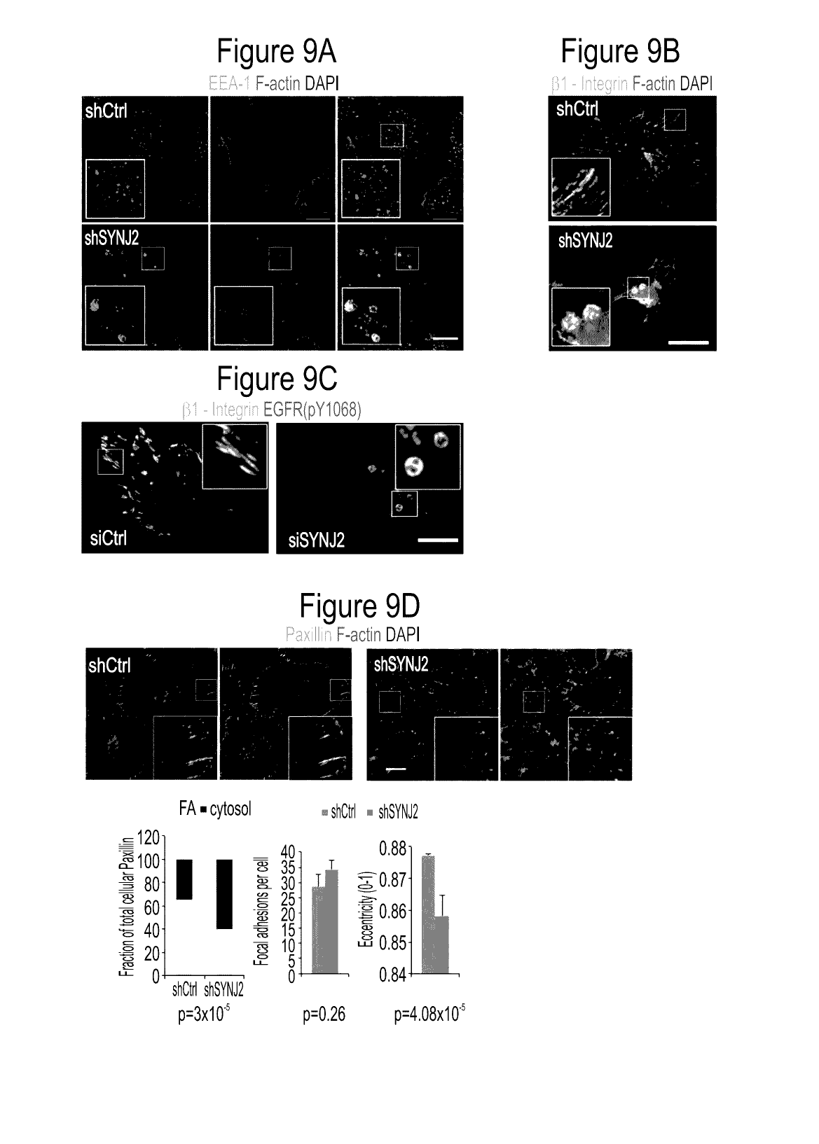

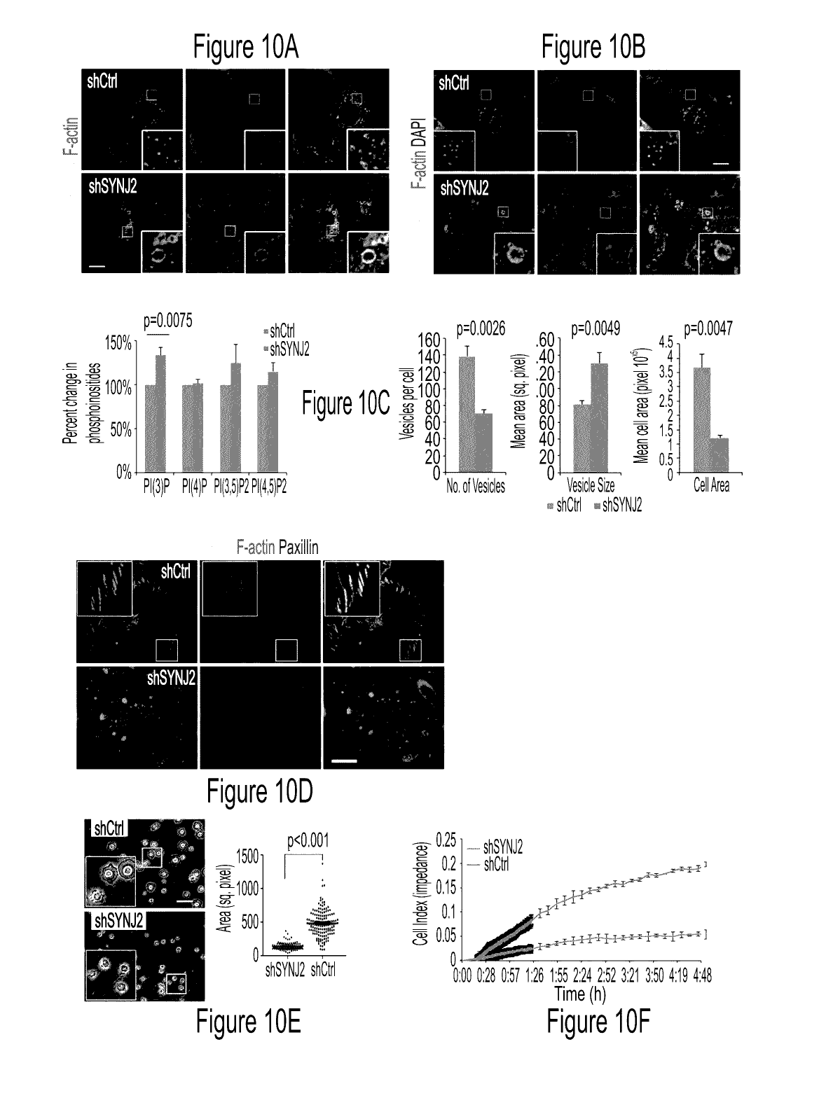

The endocytic system maintains several distinct compartments, which are defined by specific phosphoinositides (PI) [Gruenberg & Stenmark, Nat Rev Mol Cell Biol 5, 317-323 (2004)], and the present analyses uncovered strong dependency on SYNJ2. For example, by probing early endosomes for EEA1, a PI(3)P-binder, it was found that its spatial organization was markedly altered in SYNJ2-depleted cells (FIG. 9A). Similarly, probing the recycling compartment using GFP-tagged Rab4, uncovered strong associations with the circular actin patches of shSYNJ2 cells (FIG. 10A). The distribution of another marker of early endosome, Rab5, also reflected dependence on SYNJ2 (FIG. 10B). Whereas the number of Rab5-positive vesicles was significantly lower in shSYNJ2-depleted cells, their average size increased and they partly localized to circular actin patches (FIG. 9A). To uncover underlying alterations in phosphoinositides, shCtrl and shSYNJ2 MDA-MB-231 cells that were biosynthetically labeled were compared, and thereafter their phospholipids were extracted (FIG. 10C). The results showed that mainly PI(3)P, but also PI(4,5)P₂ and PI(3,5)P₂ were present at higher levels in shSYNJ2 cells, whereas PI(4)P levels remained unaltered and levels of both PI(3,4)P₂ and PI(3,4,5)P₃ were hardly detectable by this method. While these results confirm the notion that SYNJ2 targets primarily the D5 position of PIs, the present inventors assume that the rather limited global effects observed represent larger local differences. In conclusion, these observations reaffirm that SYNJ2 controls cargo sorting at the early endosome, as well as in the subsequent recycling step.

Along with recycling of RTKs like EGFR, vesicular trafficking of integrins and their interactions with downstream partners, such as Paxillin, play major roles in cell migration and focal adhesion (FA) maturation [Guo & Giancotti, Nat Rev Mol Cell Biol 5, 816-826 (2004)]. Accordingly, beta-1 integrin and phosphorylated-EGFR (pEGFR) localized to FAs of MDA-MB-231 cells. By contrast, due to abnormal accumulation in large vesicles, both proteins failed to localize to the periphery of SYNJ2-depleted cells (FIGS. 10D, S5B and S5C). Moreover, using Paxillin as a marker of mature FAs, it was found that FAs assumed a round and relatively short appearance in shSYNJ2 cells (FIG. 9D). Taken together, these observations imply that SYNJ2 is required for substrate adhesion, a scenario examined by measuring cell spreading using two methods (FIGS. 10E and 10F). The results demonstrated attenuated adhesion of shSYNJ2 cells, which is attributed to defective delivery of both integrins and RTKs to FAs.

### Example 7

### SYNJ2 Regulates the Assembly of Invadopodia

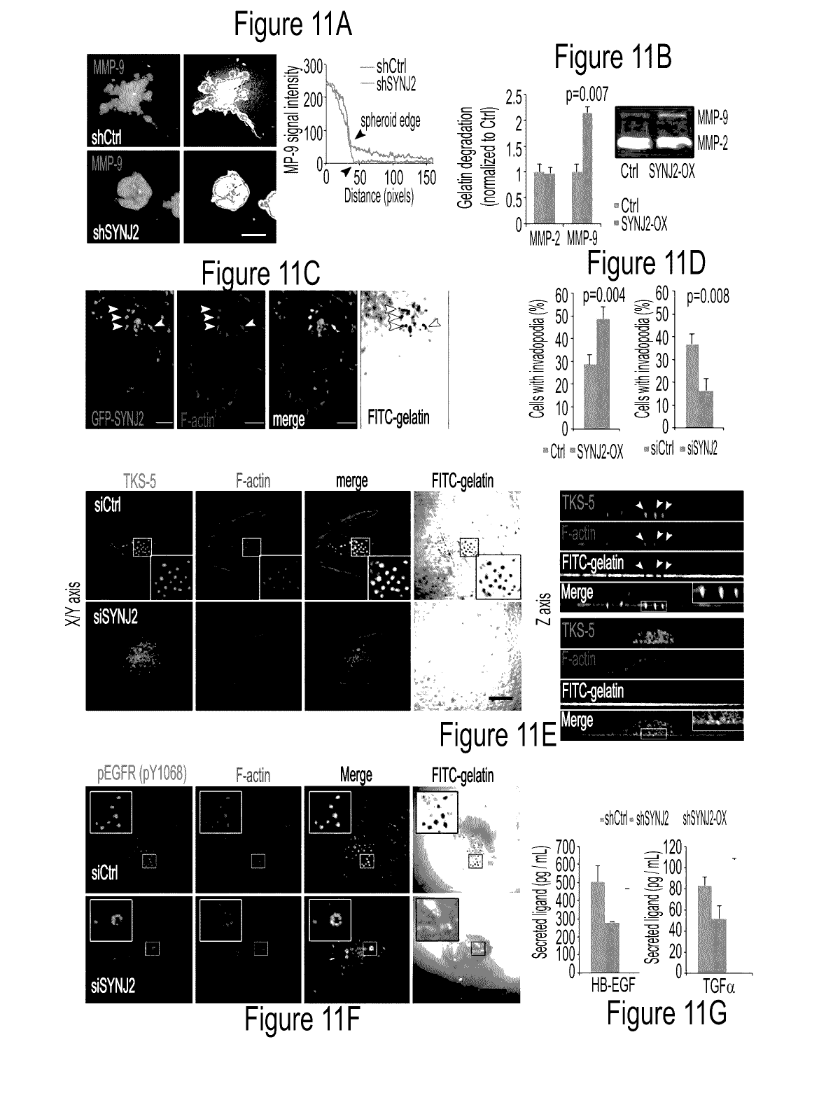

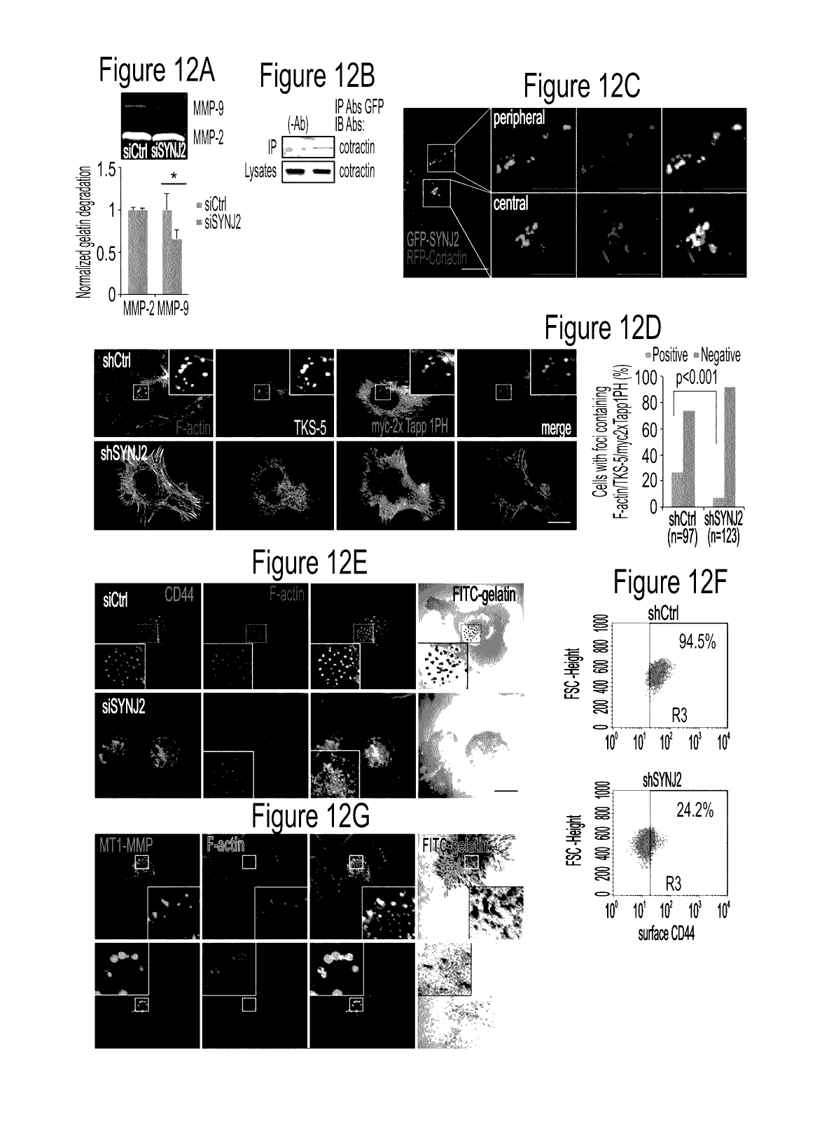

Matrix-based 3D cultures of MDA-MB-231 cells normally display wedge-shaped protrusions, but shSYNJ2 cells displayed roundish extensions (FIG. 11A), suggesting defective matrix degradation. To test this, confocal immunofluorescence images of MMP-9 were obtained, and it was noted that shSYNJ2 spheroids displayed a relatively sharp decrease of MMP-9 abundance at their borders (FIG. 11A), likely due to impaired secretion. Indeed, zymography assays performed on conditioned media confirmed defective MMP-9 secretion by cells that were treated with siSYNJ2 oligonucleotides, but MMP-2 secretion remained unaltered (FIG. 12A). Conversely, media conditioned by cells overexpressing SYNJ2 displayed a substantial increase in MMP-9 activity (FIG. 11B), in line involvement of SYNJ2 in MMP secretion.

To visualize focal proteolysis, cells were plated on cross-linked fluorescent gelatin and probed for the actin-centered, matrix-degrading organelles called invadopodia [Murphy & Courtneidge, Nat Rev Mol Cell Biol 12, 413-426 (2011)]. In line with previous reports, active matrix proteolysis corresponded to actin dots localized underneath the cell body. Importantly, SYNJ2-GFP puncta co-localized with these structures (FIG. 11C, arrowheads), which resembled the actin-associated long-lived puncta presented in FIG. 5B. Expression levels of SYNJ2 are clearly correlated with invadopodia occurrence; whereas SYNJ2 overexpression almost doubled the fraction of invadopodia-containing cells, siSYNJ2 significantly reduced the incidence of invadopodia (FIG. 11D), implying causal relationships. Next, potential physical associations between SYNJ2 and Cortactin, a well-characterized marker of invadopodia, was examined and found that SYNJ2 and Cortactin co-immunoprecipitate (FIG. 12B), as well as co-localize to both invadopodia and leading edges (FIG. 12C). To firmly establish a driving role for SYNJ2, TKS5 was observed, a PI(3,4)P₂ and a binder of Cortactin that serves as a signpost of invadopodia [Courtneidge et al., Cold Spring Harb Symp Quant Biol 70, 167-171 (2005)]. As expected, endogenous TKS5 localized to multiple ventral sites of matrix degradation in control MDA-MB-231 cells, but almost no active sites were found in siSYNJ2 cells, and TKS5 lost its ventral location (FIG. 6E; X-Y and Z panels). Furthermore, because invadopodial TKS5 anchors at PI(3,4)P₂ [Oikawa et al., J Cell Biology 182, 157-169 (2008)], a PI(3,4)P₂-binding domain, namely the PH domain of Tapp1 was used as a probe. Consistent with previous reports, ectopic expression of the PH domain reduced the number of invadopodia, but nevertheless the remaining signal co-localized with TKS5 and actin cores (FIG. 12D). In conclusion, SYNJ2 appears necessary at a step preceding TKS5 engagement, consistent with sequential action of PI3K [Yamaguchi et al., J Cell Biology 193, 1275-1288 (2011)] and SYNJ2, which respectively generate PI(3,4,5)P₃ and then PI(3,4)P₂, to anchor TKS5 at sites of EGFR-induced activation of PI3K.

In line with an EGFR-PI3K-SYNJ2 scenario, the active form of EGFR (pEGFR) was detected in proteolytically active invadopodia, but EGFRs of SYNJ2-depleted cells localized to swollen vesicles (FIG. 11F). The mechanism responsible for local receptor activation remains unknown. According to one model, cleavage of pro-ligands, such as the heparin-binding EGF (HB-EGF), by a complex comprising MMP-7 and CD44, might locally stimulate EGFR [Yu et al., Genes & Development 16, 307-323 (2002)]. In line with this model, SYNJ2 abundance was correlated with secretion of EGFR ligands (FIG. 11G), and detected co-localization of CD44 with the actin cores of invadopodia (FIG. 12E) Likewise, using flow cytometry, it was found that surface expression of CD44 was strongly suppressed in shSYNJ2 cells relative to control cells (FIG. 12F). Yet another critical step in the maturation of invadopodia is the recruitment of the membrane type-1 matrix metalloproteinase (MT1-MMP), which activates soluble MMPs [Wang & McNiven, J Cell Biology 196, 375-385 (2012)]. Accordingly, it was found that in control cells MT1-MMP corresponded to sites of invadopodial protrusions, but MT1-MMP molecules of siSYNJ2 cells formed large aggregates, which were not associated with matrix degradation (FIG. 9E). Taken together, these observations imply that SYNJ2 is essential for invadopodia priming, as well as for targeting to this organelle both proteases and two previously unrecognized residents, CD44 and an active EGFR.

### Example 8

### SYNJ2 Promotes Tumor Growth and Metastatic Spread in a Mammary Animal Model

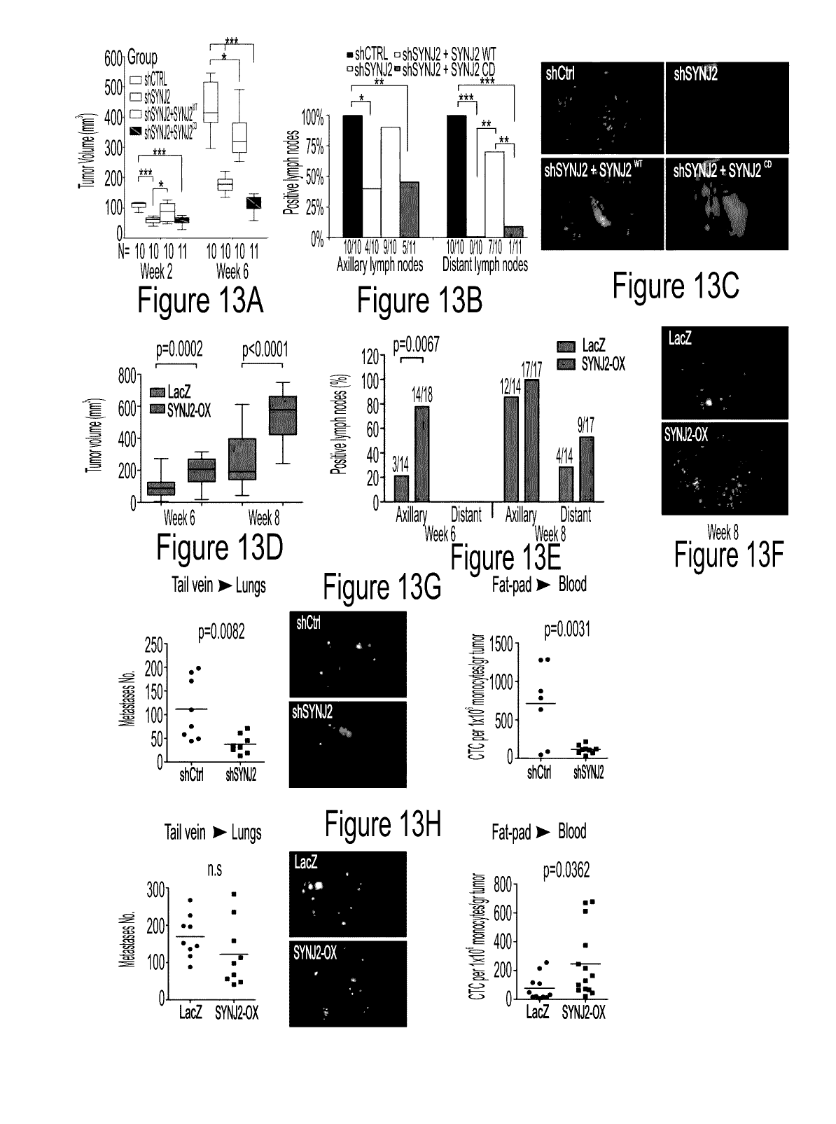

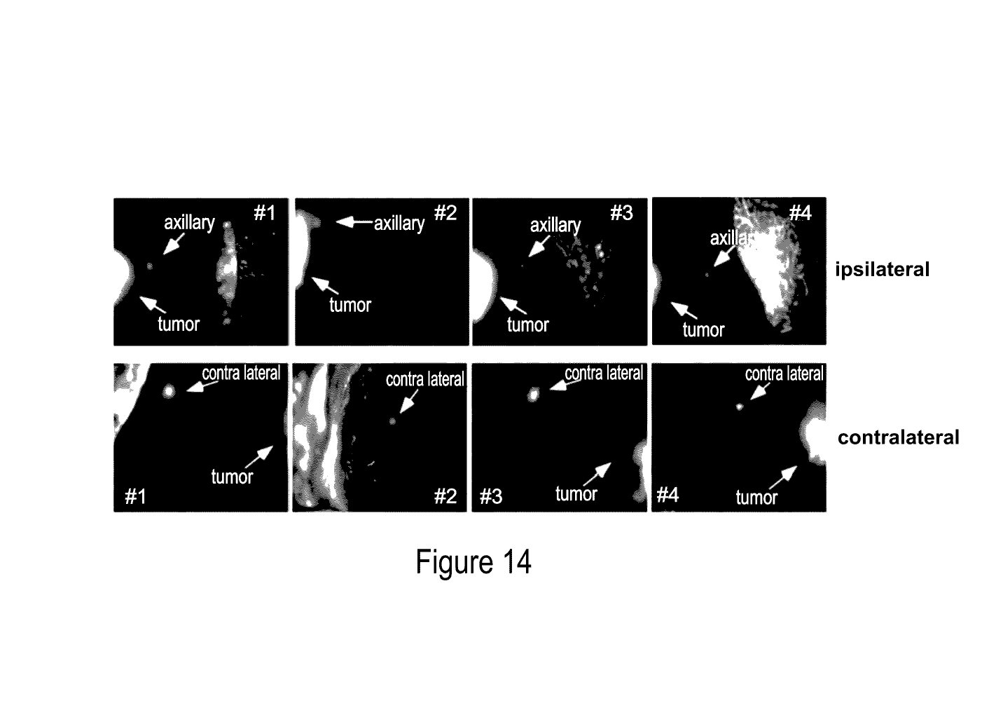

To assess the effect of SYNJ2 on metastatic dissemination in vivo MDA-MB-231-RFP cells (and derivatives) were implanted into the mammary fat pad of female mice, and two or six weeks later measured both tumor size (FIG. 13A) and metastases (FIG. 13B). Primary tumor growth was significantly faster in the shCtrl and shSYNJ2+SYNJ2^{WT} (‘active rescue’) groups, relative to the shSYNJ2 and the ‘inactive rescue’ (shSYNJ2+SYNJ2^{CD}) groups. The metastatic behavior similarly correlated with SYNJ2: the shSYNJ2 and the ‘inactive rescue’ group displayed significant reduction in metastatic spread to local and distant lymph nodes (FIGS. 13B and 14). In order to examine distant metastases, mice were sacrificed and their lungs evaluated. Lungs of animals implanted with shSYNJ2 cells, or the ‘inactive rescue’ cells, showed a dramatic reduction in the number and size of metastases, compared to animals inoculated with the shCtrl or the ‘active rescue’ cells (FIG. 13C). Taken together, these results implicate SYNJ2 in metastasis promotion.

Similarly, xenografts overexpressing SYNJ2 were monitored. As expected, SYNJ2-OX cells gave rise to faster growing tumors (FIG. 13D), and they also displayed earlier onset of nodal metastases (FIG. 13E). Consistent with robust lymphatic invasion, the lungs of animals implanted with SYNJ2-OX cells showed an increase in the number of metastases (FIG. 13F). Next, the effect of SYNJ2 on intravasation or extravasation was tested. Hence, sub-clones of MDA-MB-231-RFP cells were either directly injected into the circulation (tail vein) of female mice and scored for lung colonization (extravasation), or they were implanted in the fat pad and scored in blood as circulating tumor cells (CTCs; intravasation). Note that these experiments took into account the size differences between the respective primary tumors. The normalized results indicated that SYNJ2 is necessary for both intravasation (p=0.0031) and extravasation (p=0.0082; FIG. 13G). This conclusion was further tested by using GFP-SYNJ2 overexpressing cells (FIG. 13H). Notably, the intravasation results obtained in this experiment displayed statistical significance, but the ability of SYNJ2-OX cells to better extravasate and colonize a distant organ did not reach significance, suggesting that the observed strong effects of SYNJ2 on local and distant metastasis are primarily due to enhanced intravasation into lymph and blood vessels.

### Example 9

### SYNJ2 is Associated with Aggressive Human Breast Tumors

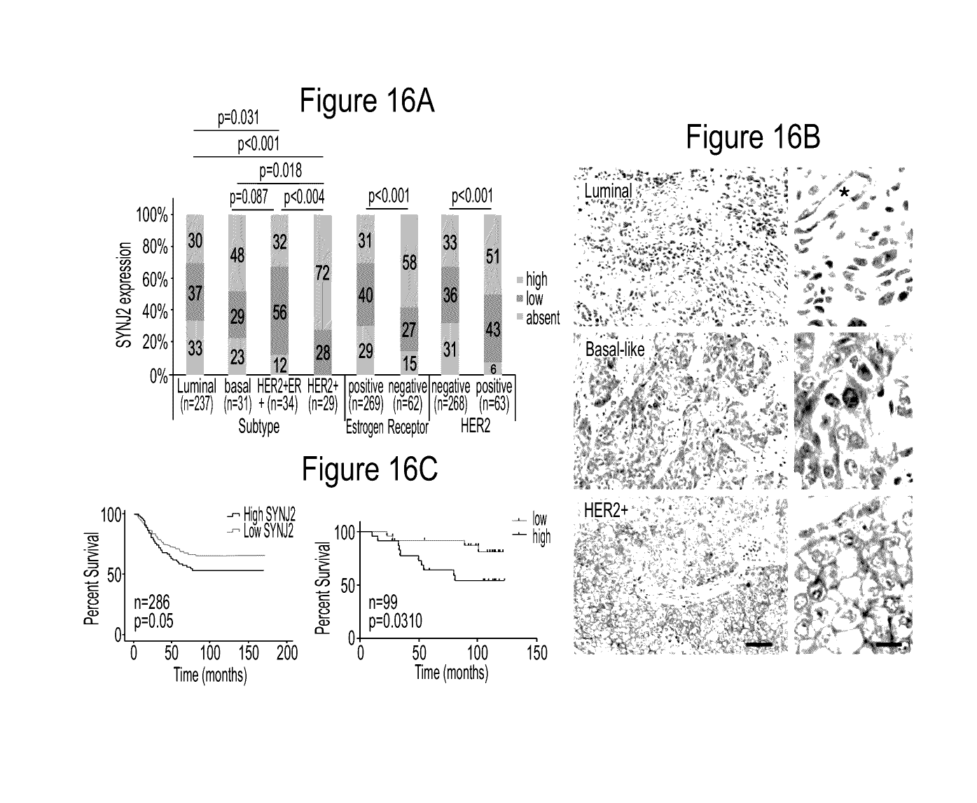

To address SYNJ2's relevance to human cancer, the transcript levels of SYNJ2 were analyzed in the NCI-60 panel of 60 human cancer lines. In line with contribution to motile phenotypes, it was found that high transcript levels of SYNJ2 associate with mesenchymal phenotypes. Next, a set of 331 paraffin-embedded samples of breast carcinomas NJ2 were immunostained (FIG. 16A). Importantly, expression intensity of SYNJ2 was positively associated with prognostically unfavorable subtypes defined by HER2 overexpression (p<0.001) and/or lack of estrogen receptor (p<0.001). However, no significant association was found between SYNJ2 abundance and age, histological subtype, axillary lymph node status, and differentiation grade. Interestingly, staining patterns for SYNJ2 also varied; whereas HER2+ tumors exhibited mostly membranal staining, luminal and triple negative tumors displayed cytoplasmic staining (FIG. 16B). To support the findings, SYNJ2 mRNA levels were analyzed in two cohorts of breast cancer specimens and an association was found with shorter patient survival rates (FIG. 16C). Altogether, these observations support involvement of SYNJ2 in progression of breast cancer, but they leave open the mechanism behind transcript up-regulation.

In summary, the observations made in animals, along with the clinical data and the in vitro experiments, clearly indicate that dephosphorylation of inositol lipids by SYNJ2 is critical for the metastatic process, primarily because of the cardinal roles played by phosphoinositides in trafficking of cell surface molecules to and from invadopodia and the leading edge. Below is presented a working model (FIG. 15) and discuss the multiple functions of SYNJ2 in the broad context of tumor progression.

### Example 10

### Selective Inhibitors of the 5′-Phosphatase Activity of SYNJ2

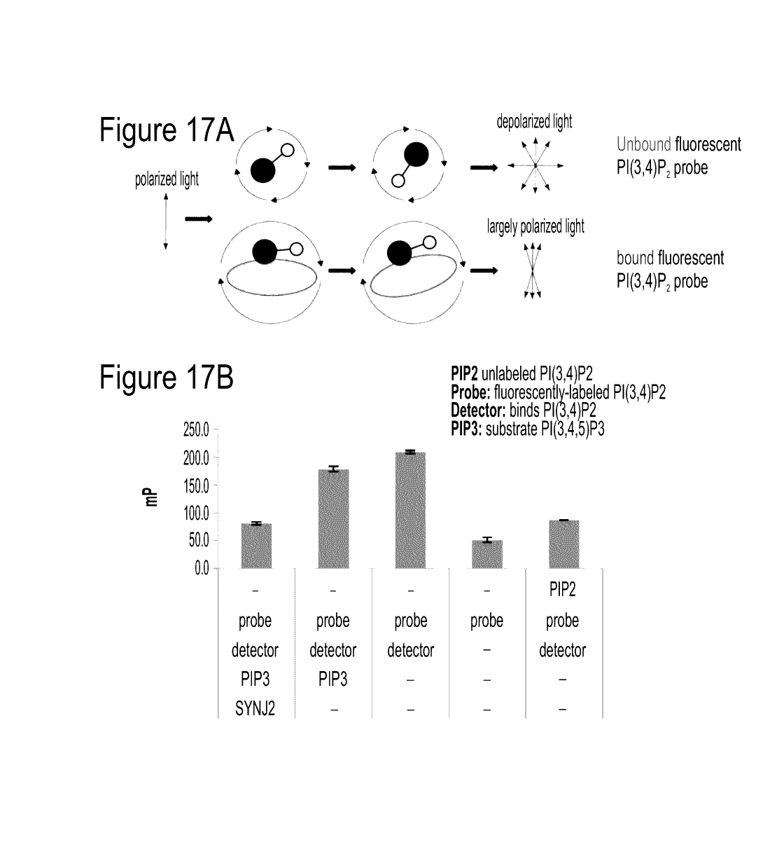

In order to identify selective inhibitors of SYNJ2 phosphatase activity, a fluorescence polarization competitive assay was used, based on the principle that when excited by polarized light, fluorescent molecules bound to a larger element (e.g., a protein) exhibit more polarized fluorescence than do free, rapidly rotating fluorescent molecules in solution. Addition of detector molecule (e.g., a binding protein) that binds a fluorescent probe therefore increases the polarization readings for fluorescent measurements of a solution (see FIG. 17A).

This assay was used to screen libraries of test compounds obtained from AnalytiCon and ChemBridge.

In the performed screen, the present inventors measured, in the presence of different test compounds, the enzymatic de-phosphorylation by SYNJ2 or SYNJ1 of the 5′ position of PI(3,4,5,)P3 to produce PI(3,4)P2. Once the enzymatic reaction was completed/stopped, the solution containing the PI(3,4)P2 products was mixed with a mixture of PI(3,4)P2 binding protein (detector) and a fluorescent PI(3,4)P2 (probe). The detector protein used was the purified PH-domain of Tapp1 that selectively binds PI(3,4)P2. As demonstrated in FIG. 17B, the polarization values measured in this assay decreased as the bound PI(3,4)P2 fluorescent probes were being displaced by un-labeled PI(3,4)P2 produced by the enzymatic activity of SYNJ2 or SYNJ1, resulting in an increase in unbound fluorescent probe.

The assay was performed using different concentrations of each test compound, and IC50 values were calculated from the data for different concentrations of test compound.

As shown in Table 1, the assay identified 24 compounds capable of inhibiting PI(3,4)P2 production by SYNJ2.

TABLE 1
Selective SYNJ2 inhibitory compounds

| | AnalytiCon | | | |
|---|---|---|---|---|
| | Cat. No. | | IC50 | |
| No. | [CAS No.] | Compound name | (μM) | Structure |
| 1 | NP-001872 | Ampelopsin | 3.17 | 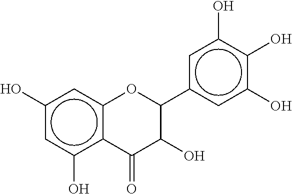 |
| 2 | NP-003195 | 3-Hydroxy-5- phenylpentanoic acid 3-D- glucopyranoside | 1.87 | 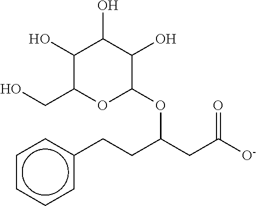 |
| 3 | NP-012228 | Cedeodarin | 2.19 | 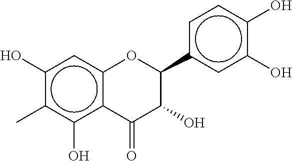 |
| 4 | NP-014109 | (−)-Catechin-7- gallate | 3.44 | 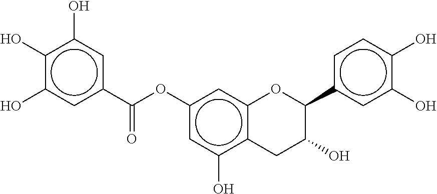 |
| 5 | NP-015304 | Paconivayin | 6.02 | 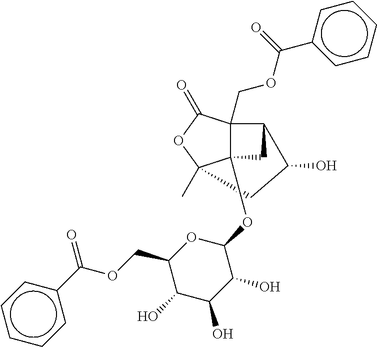 |
| 6 | NP-003491 | Sericoside | 3.52 | 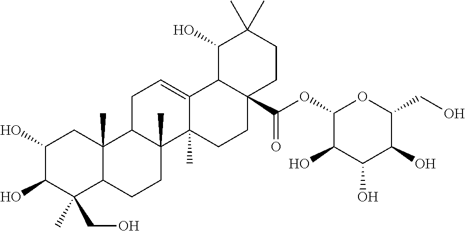 |
| 7 | NP-000303 | Keioside | 3.4 | 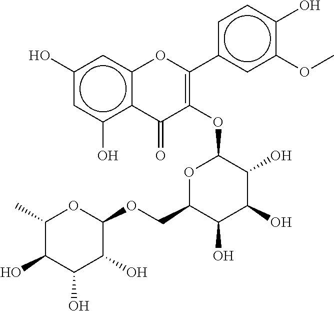 |
| 8 | NP-005201 | 2,4,3′,4′- tetrahydroxy-6- methoxy- benzophenone | 2.84 | 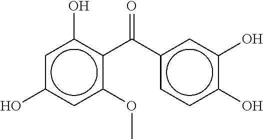 |
| 9 | NP-001585 | 2-Hydroxy-1,8- cineole- glucopyranoside | 1.98 | 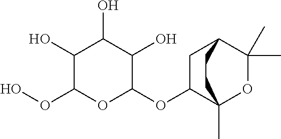 [SMILES: CC1(C)O[C@@]2(C)CC[C@@H]1CC2OC1OC(OO)C(O)C(O)C1O] |
| 10 | NP-014110 | (−)-Gallocatechin- 7-gallate | 1.075 | 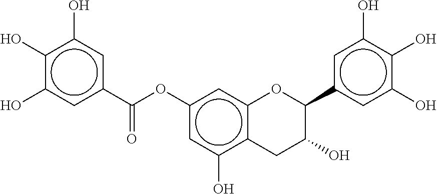 |
| 11 | NP-002326 | (−)-Epicatechin-3- gallate | 2.88 | 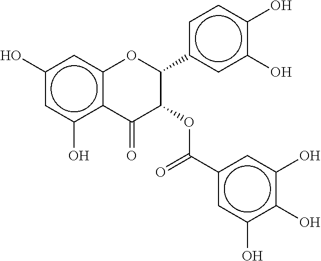 |
| 12 | NP-000360 | | 1.67 | 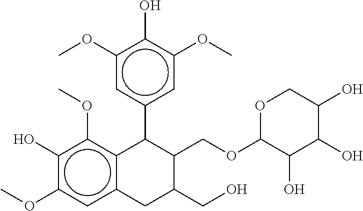 |
| 13 | NP-002973 | 1,6- Digalloylglucose | 7.53 | 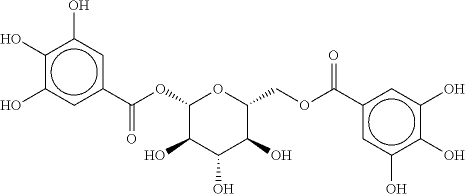 |
| 14 | NP-015222 | 3,7-dimethyloct- 2-ene-1,4,7-triol 1-β-D- glucopyranoside | 1.24 | 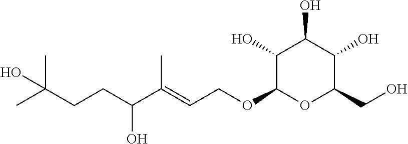 [SMILES: C/C(=C\CO[C@@H]1O[C@H](CO)[C@@H](O)[C@H](O)[C@H]1O)C(O)CCC(C)(C)O] |
| 15 | NP-000181 | Pyracanthoside | 0.874 | 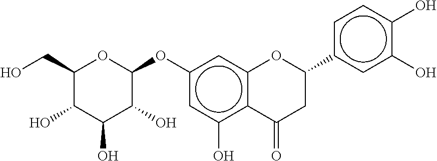 |
| 16 | NP-008708 | Isobiflorin | 2.71 | 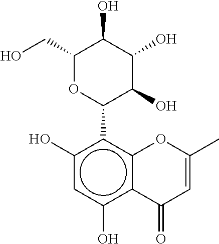 |
| 17 | [CAS: 55-56-1] | Chlorhexidine | 10 | 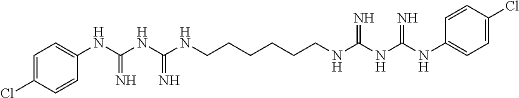 [SMILES: N=C(NCCCCCCNC(=N)NC(=N)Nc1ccc(Cl)cc1)NC(=N)Nc1ccc(Cl)cc1] |
| 18 | [CAS: 3546-41-6] | Pyrvinium pamoate | 1.25 | 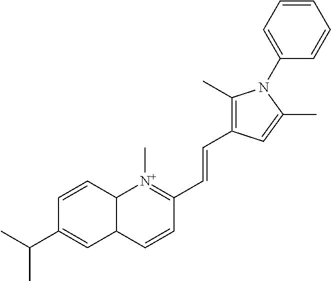 [SMILES: Cc1cc(/C=C/C2=[N+](C)C3C=CC(C(C)C)=CC3C=C2)c(C)n1-c1ccccc1] |
| 19 | NP-003299 | Quercitrin gallate | 2.94 | 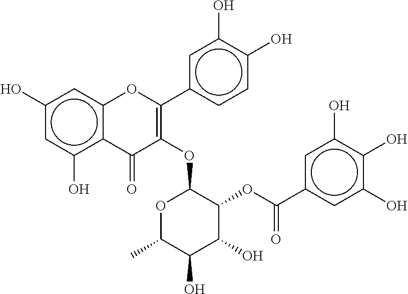 |
| 20 | NP-013254 | 4′-O-Methyl-3,5- di-O- caffeoylquinic acid | 3.17 | 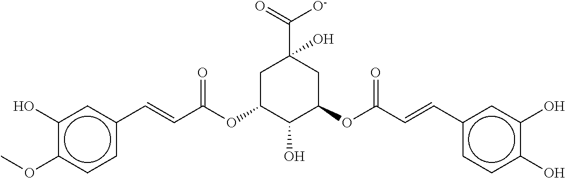 |
| 21 | NP-012429 | 3,5-Di-O- galloylshikimic acid | 3.75 | 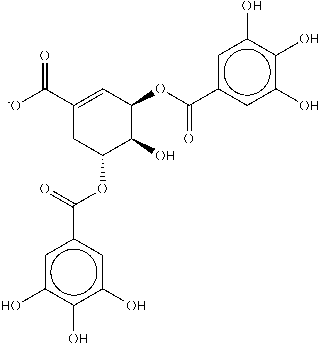 |
| 22 | NP-002325 | Epiafzelechin | 15.9 | 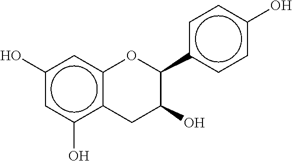 |
| 23 | NP-003143 | Oxyresveratrol | 6.65 | 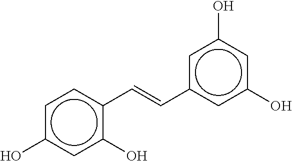 |
| 24 | NP-012649 | 6,8-di-(2,3- dihydroxybenzyl)- pinocembrin | 10.3 | 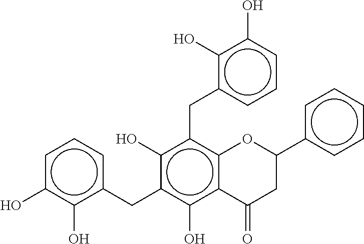 |

For each of the identified compounds except for Compound 18 (pyrvinium pamoate), the IC50 for inhibition of SYNJ1 was higher (at least 15.9 μM) than that of the IC50 for inhibition of SYNJ2, indicating that the compounds selectively inhibited SYNJ2.

Compound 18 exhibited an IC50 of 1.0 μM for inhibition of SYNJ1. Binding of Compound 18 to SYNJ2 was confirmed by microscale thermophoresis (data not shown).

The results in Table 1 indicate that molecules comprising hydroxylated rings (particularly with more than one hydroxy group), such as phenol and/or monosaccharide moieties, selectively inhibit SYNJ2 enzyme activity.

### Example 11

### Effect of SYNJ2 Inhibitors on Cell Invasion and Migration

The effect of Compounds 3, 8, 12, 14, 15, 16 and 18 on cell invasion was determined using an invasion assay performed using MDA-MB-231 breast cancer cells and BioCoat Matrigel Chambers, as described in Example 1. The compounds were assayed at a concentration of 10 μM, except for Compound 18, which was toxic to the cells at a concentration of 10 μM was assayed at a concentration of 1 or 2 μM.

As shown in FIG. 19, 2 μM Compound 18 (pyrvinium) considerable inhibited invasion of the surrounding matrix by the MDA-MB-231 cells

Similarly, each of Compounds 3, 8, 12, 14, 15 and 16 inhibited cell invasion at a concentration of 10 μM (data not shown).

The effect of Compound 18 on cell migration was further determined using a migration assay performed on MCF10A cells in a Boyden chamber. The MCF10A cells were assayed for migration or invasion in the presence of various concentrations of Compound 18 (and 10 ng/ml EGF). Cells that reached the filter bottom after 18 hours were stained and quantified.

As shown in FIGS. 20A and 20B, Compound 18 reduced cell migration in a dose-dependent manner, exhibiting an EC50 of about 0.8 μM.

These results indicate that the SYNJ2 inhibitors described herein are effective at inhibiting cancer cell invasion and migration.

### Example 12

### Attenuation of Cancer by Selective SYNJ2 Inhibitors

In order of assess the anti-cancer effect of selective SYNJ2 inhibitors, Compound 12 (also referred to herein as “NP-360” or “NP-000360”) was tested in vivo. MDA-MB-231 breast cancer cells expressing GFP (green fluorescent protein) were implanted (2.5×10⁶ per mouse) in the fat pad of 20 SCID mice (aged 6 weeks old). After tumors reached a volume of approximately 3×3×3 mm³, mice were randomized into two groups, and Compound 12 (5 mg/kg) or carrier (DMSO) were injected twice a week for 6 weeks. Tumor mass was then determined.

As shown in FIG. 21, Compound 12 (NP-360) attenuated tumor growth in comparison to the control group in a statistically significant manner (p=0.0372).

In addition, the effect of Compound 2 (also referred to herein as “NP-3195” or “NP-003195”) on tumor mass was tested in vivo, as described hereinabove for Compound 12. The effect of Compound 2 on lung metastasis was also tested in vivo, according to procedures such as described in Example 1.

As shown in FIG. 22, Compound 2 modestly reduced attenuated tumor growth in comparison to the control group.

As shown in FIGS. 23A and 23B, Compound 2 considerably reduced tumor metastasis in comparison to the control group.

These results indicate that SYNJ2 inhibitors can be effective at inhibiting tumor growth and/or metastasis in vivo.

### Example 13

### Effect of SYNJ2 Inhibitor on EGF Receptor Distribution

In order to evaluate the mechanism by which SYNJ2 inhibitors affect cells, MDA-MB-231 cells were immunoblotted with antibodies against EGFR (endothelial growth factor receptor) treated with 0.78 μM of Compound 18 (pyrvinium) or with carrier (DMSO). For comparison, the same immunoblotting was performed on cells subjected to SYNJ2 knockdown and control knockdown.

As shown in FIG. 24, treatment with 0.78 μM of Compound 18 resulted in EGFR being localized within cells, as opposed to being localized on the surface of control cells. As further shown therein, the effect of Compound 18 was similar to that of SYNJ2 knockdown, which also resulted in localization of EGFR within cells.

These results indicate that SYNJ2 inhibitors affect cells, at least in part, by causing less EGFR to be present on the surface of cells, thereby reducing their ability to bind EGF (endothelial growth factor).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-50. (canceled)

51. A method of inhibiting synaptojanin-2, the method comprising contacting the synaptojanin-2 with an effective amount of a compound having the general formula I:
X-L-[(Y)i-(Z)j]-(L-X)k Formula I
or a pharmaceutically acceptable salt thereof, wherein:
i, j and k are each independently 0 or 1, wherein at least one of i, j and k is 1;
L is absent or is a linking moiety;
X is an aryl group substituted by one or more group selected from the group consisting of hydroxy, thiohydroxy, alkoxy, aryloxy, thioalkoxy and thioaryloxy;
Z is selected from the group consisting of a monosaccharide moiety, a disaccharide moiety, a shikimate moiety and a quinate moiety; and
Y is a bicyclic moiety having the general formula II: 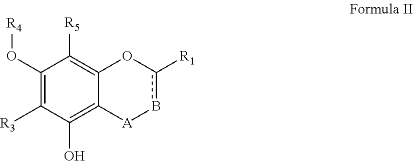
wherein:
A is absent or is CH₂, C═O, C═S or C═NR₆;
B is absent or is O, S, NR₇, CH, CH₂, C—O—R₂, C—S—R₂, C—N(R₈)—R₂, CH—O—R₂, CH—S—R₂ or CH—N(R₉)—R₂;
R₁-R₅ are each independently selected from the group consisting of hydrogen, methyl, aryl and a covalent bond with an L, Z or X moiety described herein;
R₆-R₉ are each independently selected from the group consisting of hydrogen and alkyl; and
the dashed line denotes a saturated or unsaturated bond, wherein when the dashed line denotes a saturated bond, B is O, CH₂ or CH—O—R₂, and when the dashed line denotes an unsaturated bond, B is CH or C—O—R₂,
thereby inhibiting synaptojanin-2.

52. The method of claim 51, being for preventing tumor metastasis in a subject in need thereof, the method comprising administering to said subject a therapeutically effective amount of said compound.

53. The method of claim 51, being for treating cancer in a subject in need thereof, the method comprising administering to said subject a therapeutically effective amount of said compound and an inhibitor of a cell surface receptor associated with an onset or progression of cancer.

54. The method of claim 51, wherein A is CH₂ or C═O.

55. The method of claim 51, wherein B is CH, CH₂, C—O—R₂, or CH—O—R₂.

56. The method of claim 51, wherein L is absent or is a linking moiety selected from the group consisting of C(═O), CH═CH, CH═CH—C(═O) and CH₂.

57. The method of claim 51, wherein said aryl is a phenyl.

58. The method of claim 51, wherein said Z is a monosaccharide or disaccharide, being attached to said Y, said L or said X via a glycosidic bond.

59. The method of claim 51, wherein a sum of i, j and k is 2.

60. A kit for the treatment of cancer or prevention of cancer metastasis, comprising a packaging material packaging a compound as described in claim 51 and an inhibitor of a cell surface receptor associated with an onset or progression of cancer.

61. A method of inhibiting synaptojanin-2, the method comprising contacting the synaptojanin-2 with an effective amount of a compound having the general formula III: 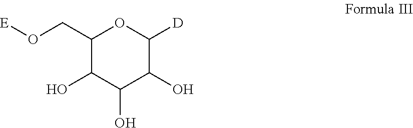
or a pharmaceutically acceptable salt thereof, wherein:
D is selected from the group consisting of: 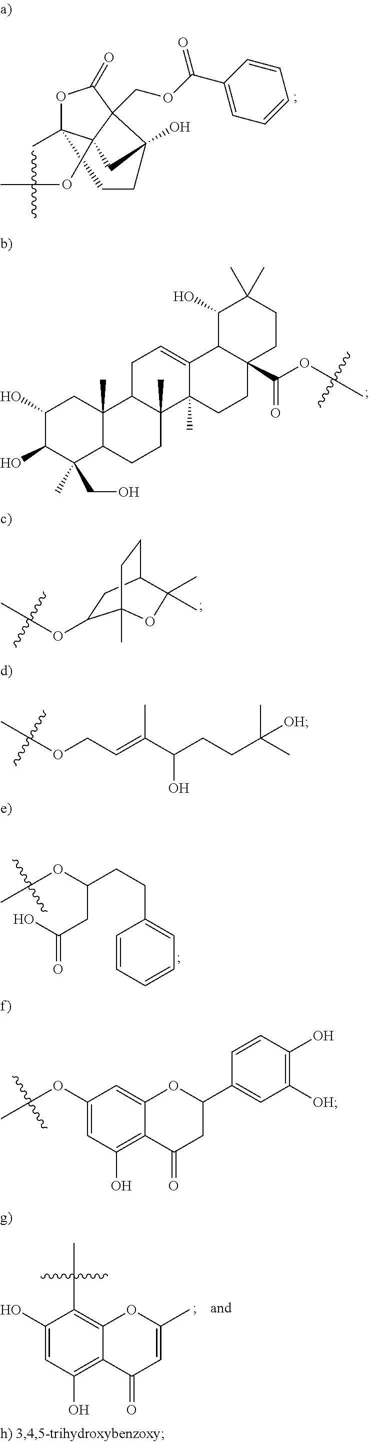
and
E is selected from the group consisting of hydrogen and substituted or non-substituted benzyl,
thereby inhibiting synaptojanin-2.

62. The method of claim 61, being for preventing tumor metastasis in a subject in need thereof, the method comprising administering to said subject a therapeutically effective amount of said compound.

63. The method of claim 61, being for treating cancer in a subject in need thereof, the method comprising administering to said subject a therapeutically effective amount of said compound and an inhibitor of a cell surface receptor associated with an onset or progression of cancer.

64. The method of claim 61, wherein the compound has the general formula: 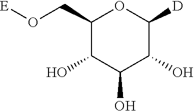
or a pharmaceutically acceptable salt thereof.

65. The method of claim 61, wherein E is hydrogen.

66. A kit for the treatment of cancer or prevention of cancer metastasis, comprising a packaging material packaging a compound as described in claim 61 and an inhibitor of a cell surface receptor associated with an onset or progression of cancer.

67. A method of inhibiting synaptojanin-2, the method comprising contacting the synaptojanin-2 with an effective amount of a compound selected from the group consisting of the compounds listed in Table 1, chlorhexidine and pyrvinium, and pharmaceutically acceptable salts thereof, thereby inhibiting synaptojanin-2.

68. The method of claim 67, wherein said compound is selected from the group consisting of Compound 12 in Table 1, chlorhexidine and pyrvinium, and pharmaceutically acceptable salts thereof.

69. The method of claim 67, being for preventing tumor metastasis in a subject in need thereof, the method comprising administering to said subject a therapeutically effective amount of said compound.

70. The method of claim 67, being for treating cancer in a subject in need thereof, the method comprising administering to said subject a therapeutically effective amount of said compound and an inhibitor of a cell surface receptor associated with an onset or progression of cancer.

71. A kit for the treatment of cancer or prevention of cancer metastasis, comprising a packaging material packaging a compound as described in claim 67 and an inhibitor of a cell surface receptor associated with an onset or progression of cancer.


SEQUENCE LISTING (4 sequences)

SEQ ID NO: 1 - RNA, 19 nt, Artificial sequence
  Example of an siRNA sequence that successfully downregulates SYNJ2 mRNA levels
ggacagcacugcagguguu

SEQ ID NO: 2 - DNA, 58 bp, Artificial sequence
  Example of an shRNA sequence that successfully downregulates SYNJ2 mRNA levels
ccggccggaagaacagtttgagcaactcgagttgctcaaactgttcttccggtttttg

SEQ ID NO: 3 - DNA, 738 bp, Artificial Sequence
  Recombinant TAPP1 PH domain derived from Homo sapiens
atggattacaaggacgacgacgataagggcggatccccttactttactcctaaaccacctcaagatagtacggttatcaaagctggatattgtgtaaaacaaggagcagtgatgaaaaactggaagagaagatattttcaattggatgaaaacacaataggctacttcaaatctgaactggaaaaggaacctcttcgcgtaataccacttaaagaggttcataaagtccaggaatgtaagcaaagcgacataatgatgagggacaacctctttgaaattgtaacaacgtctcgaactttctatgtgcaggctgatagccctgaagagatgcacagttggattaaagcagtctctggcgccattgtagcacagcggggtcccggcagatctgcgtcttctgagcatccccccggtccttcagaatccaaacacgctttccgtcctaccaacgcagccaccgccacctcacattccacagcctctcgcagcaactctttggtctcaacctttaccatggagaagcgaggattttacgagtctcttgccaaggtcaagccagggaacttcaaggtccagactgtctctccaagagaaccagcttccaaagtgactgaacaagctctgttaagacctcaaagtaaaaatggccctcaggaaaaagattgtgacctagtagacttggacgatgcgagccttccggtcagtgacgtgctcgagcaccaccaccaccaccactga

SEQ ID NO: 4 - protein, 245 aa, Artificial Sequence
  Recombinant TAPP1 PH domain derived from Homo sapiens
MDYKDDDDKGGSPYFTPKPPQDSTVIKAGYCVKQGAVMKNWKRRYFQLDENTIGYFKSELEKEPLRVIPLKEVHKVQECKQSDIMMRDNLFEIVTTSRTFYVQADSPEEMHSWIKAVSGAIVAQRGPGRSASSEHPPGPSESKHAFRPTNAATATSHSTASRSNSLVSTFTMEKRGFYESLAKVKPGNFKVQTVSPREPASKVTEQALLRPQSKNGPQEKDCDLVDLDDASLPVSDVLEHHHHHH